(19) United States
(12) Patent Application Publication
(10) Pub. No.: US 2026/0022246 A1
(43) Pub. Date: Jan. 22, 2026

(54) COLORING AGENT COMPOSITION, FILM, OPTICAL FILTER, AND NEAR INFRARED ABSORBING COMPOUND

(71) Applicant: FUJIFILM Corporation, Tokyo (JP)

(72) Inventors: Yusuke SAKAI, Shizuoka (JP); Satoru NOZAWA, Shizuoka (JP); Daisuke SASAKI, Shizuoka (JP)

(73) Assignee: FUJIFILM Corporation, Tokyo (JP)

(21) Appl. No.: 19/339,353

(22) Filed: Sep. 25, 2025

Related U.S. Application Data
(63) Continuation of application No. PCT/JP2024/011700, filed on Mar. 25, 2024.

(30) Foreign Application Priority Data
Mar. 30, 2023 (JP) 2023-056456
Sep. 27, 2023 (JP) 2023-166087

(51) Int. Cl. C09B 53/02; C07C 211/54; C09B 57/00; C09D 17/00; G02B 5/22

(52) U.S. Cl. CPC C09B 53/02; C07C 211/54; C09B 57/008; C09D 17/003; G02B 5/223; C07C 2601/12

(57) ABSTRACT

Provided are a near infrared absorbing compound represented by General Formula (1), and a coloring agent composition containing the near infrared absorbing compound. In General Formula (1), R₁ to R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, and two or more of R₁ to R₈ may be linked to each other to form a ring. R₉ to R₁₃ each independently represent a monovalent substituent, and five n's each independently represent an integer of 0 to 4. m is 1 or 2, and X⁻ represents a non-nucleophilic anion.

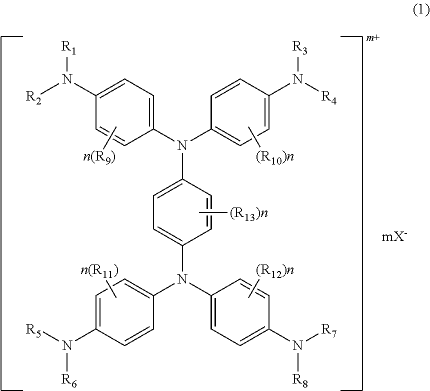

### CROSS-REFERENCE TO RELATED APPLICATIONS

This application is a continuation application of International Application No. PCT/JP2024/011700, filed Mar. 25, 2024, the disclosure of which is incorporated herein by reference in its entirety. Further, this application claims priority from Japanese Patent Applications No. 2023-056456, filed Mar. 30, 2023 and No. 2023-166087, filed Sep. 27, 2023, the disclosures of which are incorporated herein by reference in their entirety.

### BACKGROUND OF THE INVENTION

### 1. Field of the Invention

The present disclosure relates to a coloring agent composition, a film, an optical filter, and a near infrared absorbing compound.

### 2. Description of the Related Art

In recent years, various near infrared absorbing compounds or compositions containing the near infrared absorbing compound, useful for applications including a filter such as a heat ray absorbing filter, a band pass filter, and an optical filter, an ink for invisible printing, an infrared absorbing paint for preventing laser light reflection, and the like, have attracted attention.

As the above-described near infrared absorbing compound, a cyanine coloring agent, a metal complex of an oxime or a thiol, a naphthoquinone compound, a phthalocyanine compound, a naphthocyanine compound, a diimmonium compound, and the like have been known; but there is a problem in that fastness, heat stability, and the like are low.

As the compound having near infrared absorbing property and invisibility, for example, an immonium compound in which an aryl group is substituted on a terminal nitrogen, and an aminoaryl group-substituted immonium compound are known (refer to JP2002-275134A and JP2006-137935A).

In addition, as a near infrared absorbing composition having favorable light fastness and heat resistance, a compound having a diimmonium skeleton is proposed, and a compound in which an aryl group is substituted on a terminal nitrogen is also exemplified (refer to JP2017-116775A). Here, only a monovalent chloride ion and a fluoride ion are exemplified as a counter anion.

### SUMMARY OF THE INVENTION

However, since the aryl group included in the immonium compounds disclosed in JP2002-275134A and JP2006-137935A is an aryl group substituted with an amino group, the compound has multiple oxidation numbers of 1 to 6, which makes it difficult to isolate the compound in a form of a compound with a fixed oxidation number. In addition, there is a problem in that an absorption spectrum changes due to a change in oxidation number over time in practical use.

In the immonium compound disclosed in JP2017-116775A, examples of the counterion include an anion such as a monovalent chloride ion and a fluoride ion, but there is a concern that the diimmonium compound having the exemplified nucleophilic anion may undergo a decomposition reaction at normal temperature. Therefore, there is a problem in that the practical use is not suitable from the viewpoint of stability.

An object to be achieved by one embodiment of the present disclosure is to provide a coloring agent composition containing a compound which has absorption in a long-wavelength infrared region and is thermally stable, a film which is a cured substance of the coloring agent composition, and an optical filter including the film.

An object to be achieved by another embodiment of the present disclosure is to provide an infrared absorbing compound which has absorption in a long-wavelength infrared region and is thermally stable.

Methods for achieving the objects described above include the following aspects.
<1> A coloring agent composition comprising:
a compound represented by General Formula (1).

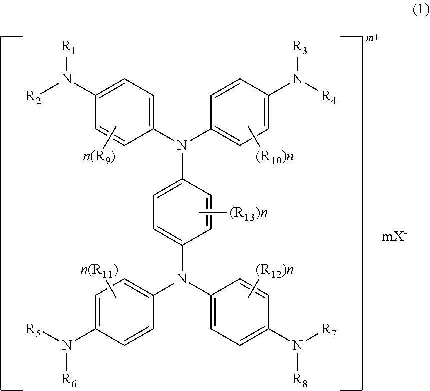

In General Formula (1), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring. In General Formula (1), R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other,
m is 1 or 2, and X⁻ represents a non-nucleophilic anion.
<2> A coloring agent composition comprising:
a compound represented by General Formula (2).

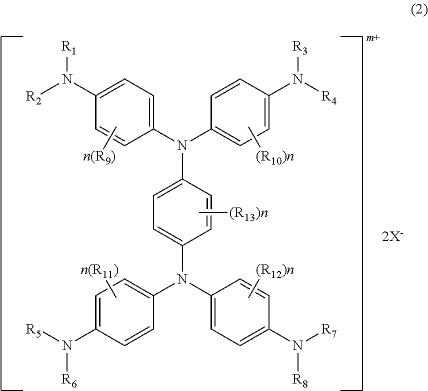

In General Formula (2), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring. In General Formula (2), R₉, R₁₀, R₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other, and
X⁻ represents a non-nucleophilic anion.
<3> The coloring agent composition according to <1>or <2>,
in which the coloring agent composition is an image forming material.
<4> The coloring agent composition according to <1>or <2>, further comprising:
a resin.
<5> A film comprising:
the coloring agent composition according to <4>.
<6> An optical filter comprising:
the film according to <5>.
<7> A near infrared absorbing compound represented by General Formula (1).

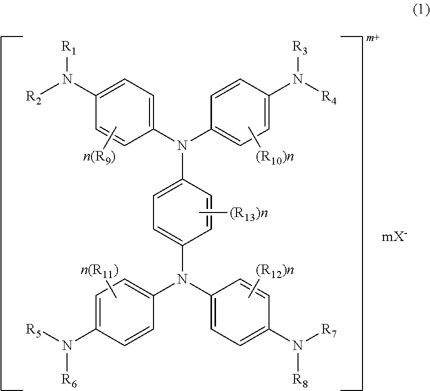

In General Formula (1), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring. In General Formula (1), R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other,
m is 1 or 2, and X⁻ represents a non-nucleophilic anion.
<8> A near infrared absorbing compound represented by General Formula (2).

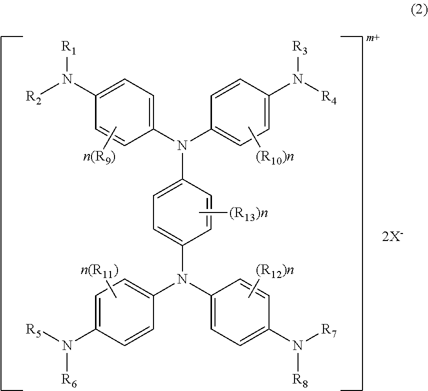

In General Formula (2), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring. In General Formula (2), R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other, and
X⁻ represents a non-nucleophilic anion.

According to one embodiment of the present disclosure, there are provided a coloring agent composition containing a compound which has absorption in a long-wavelength infrared region and is thermally stable, a film which is a cured substance of the coloring agent composition, and an optical filter including the film.

According to another embodiment of the present disclosure, there is provided an infrared absorbing compound which has absorption in a long-wavelength infrared region and is thermally stable.

### BRIEF DESCRIPTION OF THE DRAWINGS

FIG. 1 is an absorption spectrum of Exemplary Compound A-1 in a chloroform solution.

FIG. 2 is an absorption spectrum of Exemplary Compound B-1 in a chloroform solution.

### DESCRIPTION OF THE PREFERRED EMBODIMENTS

Hereinafter, the contents of the present disclosure will be described in detail.

In the present disclosure, a “total solid content” refers to a total mass of components obtained by removing a solvent from the whole composition of the composition. In addition, the “solid content” refers to components excluding the solvent as described above, and the components may be, for example, a solid or a liquid at 25° C.

Regarding a term, group (atomic group) of this present disclosure, a term with no description of “substituted” and “unsubstituted” includes both a group not including a substituent and a group including a substituent. For example, an “alkyl group” includes not only an alkyl group having no substituent (unsubstituted alkyl group), but also an alkyl group having a substituent (substituted alkyl group).

In the present disclosure, unless specified otherwise, “exposure” denotes not only exposure using light but also drawing using a corpuscular beam such as an electron beam or an ion beam. In addition, generally, examples of light used for the exposure include actinic rays or radiation such as a bright line spectrum of a mercury lamp, far ultraviolet rays typified by an excimer laser, extreme ultraviolet rays (EUV light), X-rays, or electron beams.

In the present disclosure, “(meth)acrylate” denotes either or both of acrylate and methacrylate, “(meth)acryl” denotes either or both of acryl and methacryl, and “(meth)acryloyl” denotes either or both of acryloyl and methacryloyl.

With regard to symbols described in the general formulae of the present disclosure, “R₁, R₂, R₃, R₄, R₅, R₆, R₇, and R₈” may be abbreviated as “R_{I} to R₈” and “R₉, R₁₀, R₁₁, R₁₂, and R₁₃” may be abbreviated as “R₉ to R₁₃”.

In the present disclosure, in a chemical formula, Me represents a methyl group, Et represents an ethyl group, Pr represents a propyl group, Bu represents a butyl group, Ac represents an acetyl group, Bn represents a benzyl group, and Ph represents a phenyl group.

In the present disclosure, the term “step” is not only an independent step, but also includes a step which is not clearly distinguished from other steps in a case where an intended action of the step is obtained.

In the present disclosure, “% by mass” has the same definition as that for “% by weight”, and “part by mass” has the same definition as that for “part by weight”.

In the present disclosure, the numerical ranges shown using “to” indicate ranges including the numerical values described before and after “to” as the minimum value and the maximum value.

Furthermore, in the present disclosure, in a case where a plurality of substances corresponding to each component are contained in the composition, the amount of each component contained in the composition means the total amount of the plurality of substances, unless otherwise specified.

In a numerical range described in a stepwise manner in the present disclosure, an upper limit or a lower limit described in one numerical range may be replaced with an upper limit or a lower limit in another numerical range described in a stepwise manner. In addition, in a range of numerical values described in the present disclosure, the upper limit value or the lower limit value of the range of numerical values may be replaced with values shown in the examples.

Furthermore, in the present disclosure, a combination of two or more preferred aspects is the more preferred aspects.

In the present disclosure, a long-wavelength infrared region refers to a wavelength range of 1,150 nm to 2,000 nm. Hereinafter, an infrared absorbing compound having absorption in the wavelength range of 1,150 nm to 2,000 nm may be referred to as “near infrared absorbing compound”.

Unless otherwise specified, a transmittance in the present disclosure is a transmittance at 25° C.

In the present disclosure, a weight-average molecular weight and a number-average molecular weight of a resin are defined as values in terms of polystyrene measured by gel permeation chromatography (GPC).

In the present disclosure, in a case where the content of a “structural unit” in a resin is defined by a molar ratio, the “structural unit” is synonymous with the “monomer unit”. However, the “monomer unit” in the present disclosure may be modified after polymerization by a polymer reaction or the like.

“Room temperature” means an atmosphere temperature which is not particularly controlled, and in the present disclosure, the “room temperature” means “25° C.” unless otherwise specified.

### Coloring Agent Composition

A first embodiment of the coloring agent composition according to the present disclosure (hereinafter, also simply referred to as “composition (1)”) contains a compound represented by General Formula (1) (hereinafter, also simply referred to as “specific compound (1)”).

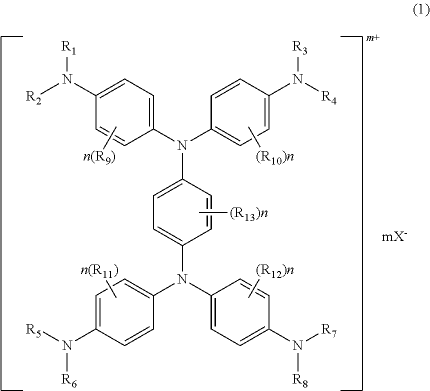

In General Formula (1), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where two or more of R₁, R₂, R₃, R₄, R₅, R₆, R₇, and R₈ may be linked to each other to form a ring.

In a case where R₁, R₂, R₃, R₄, R₅, R₆, R₇, and R₈ represent a group selected from a phenyl group having a substituent in which a σp value on Hammett's rule is −0.5 or more or an aryl group having 7 to 20 carbon atoms, from the viewpoint of stability of the specific compound (1), it is preferable to be a phenyl group having a substituent in which a σp value on Hammett's rule is −0.5 or more or an aryl group having 10 to 20 carbon atoms, which may have a substituent; it is more preferable to be a phenyl group having a substituent in which a σp value on Hammett's rule is −0.5 or more, a naphthyl group which may have a substituent, or an anthryl group which may have a substituent; and it is still more preferable to be a phenyl group having a substituent in which a σp value on Hammett's rule is −0.5 or more.

Among these, from the viewpoint of synthetic suitability, an aspect in which all of R₁, R₂, R₃, R₄, R₅, R₆, R₇, and R₈ in General Formula (1) are a phenyl group having a substituent in which a σp value on Hammett's rule is −0.5 or more is preferable.

The substituent of the phenyl group is a substituent having a σp value on Hammett's rule of −0.5 or more. Here, the Hammett's substituent constant σ value will be described. The Hammett's rule is an empirical rule advocated by L. P. Hammett in 1935 so as to quantitatively discuss the effect of substituent on the reaction or equilibrium of benzene derivatives and its propriety is widely admitted at present.

Substituent constants obtained by the Hammett's rule are a σp value and a om value, and these values can be found in many general books. For example, these values are specifically described in “Lange's Handbook of Chemistry”, edited by J. A. Dean, 12th edition, 1979 (McGraw-Hill), “Chemistry Region”, extra edition, No. 122, pp. 96 to 103, 1979 (Nankodo, Co., Ltd.), and Chem. Rev., 1991, Vol. 91, pp. 165 to 195.

The above-described substituent having a Hammett's substituent constant σp value of −0.5 or more in General Formula (1) indicates that the substituent is from a weakly electron donating group to an electron withdrawing group.

From the viewpoint of the stability of the specific compound (1), the σp value on Hammett's rule is preferably −0.40 or more, more preferably −0.35 or more, and still more preferably −0.30 or more.

Examples of the substituent having a σp value on Hammett's rule of −0.5 or more include a hydroxy group (−0.37), a methyloxy group (−0.27), a methyl group (−0.17), a chloro group (−0.23), a cyano group (0.66), a carboxyl group (—COOH: 0.45), an alkoxycarbonyl group such as —COOMe (0.45), an aryloxycarbonyl group such as —COOPh (0.44), a carbamoyl group such as —CONH₂ (0.36), an alkylcarbonyl group such as —COMe (0.50), an arylcarbonyl group such as —COPh (0.43), an alkylsulfonyl group such as —SO₂Me (0.72), an arylsulfonyl group such as —SO₂Ph (0.68), a trifluoromethyl group (0.54), and a nitro group (0.78). The numerical value shown in the parentheses after the substituent in the above-described examples is a σp value on Hammett's rule.

In General Formula (1), in a case where R₁, R₂, R₃, R₄, R₅, R₆, R₇, and R₈ are the phenyl group or an aryl group having 7 to 20 carbon atoms, which may have a substituent, it is preferable that two of R₁ to R₈ have the substituent having a Hammett's substituent constant op value of −0.5 or more, it is more preferable that four of R₁ to R₈ have the substituent having a Hammett's substituent constant σp value of −0.5 or more, and it is still more preferable that all of R₁ to R₈ have the substituent having a Hammett's substituent constant σp value of −0.5 or more. Among these, as described above, an aspect in which all of R₁, R₂, R₃, R₄, R₅, R₆, R₇, and R₈ are the phenyl group having a substituent in which a σp value on Hammett's rule is −0.5 or more is preferable.

The substituents having a Hammett's substituent constant σp value of −0.5 or more, which are present in a plural number, may be the same or different from each other, but from the viewpoint of synthetic suitability, it is preferable that all of the substituents are the same substituent.

In a case where R₁, R₂, R₃, R₄, R₅, R₆, R₇, and R₈ represent an aryl group having 7 to 20 carbon atoms, which may have a substituent, preferred examples of the aryl group include an aryl group having 7 to 20 carbon atoms, and more preferred examples thereof include an aryl group having 10 to 20 carbon atoms. Specific examples of the aryl group having 7 to 20 carbon atoms include a naphthyl group, an anthryl group, and a pyrenyl group.

In a case where the aryl group having 7 to 20 carbon atoms has a substituent, examples of the substituent include the same substituents as those mentioned as a monovalent substituent exemplified as R₉, R₁₀, R₁₁, R₁₂, and R₁₃ later.

Among these, from the viewpoint of synthetic suitability, the monovalent substituent is preferably a substituent selected from an alkyl group, a hydroxy group, an alkoxy group, an alkoxycarbonyl group, or an alkylcarbonyloxy group.

In a case where R₁, R₂, R₃, R₄, R₅, R₆, R₇, and R₈ represent a heteroaryl group which may have a substituent, preferred examples of the heteroaryl group include a heteroaryl group having 1 to 20 carbon atoms, and more preferred examples thereof include a heteroaryl group having 1 to 12 carbon atoms. Examples of a heteroatom included in the ring of the heteroaryl group include a nitrogen atom, an oxygen atom, and a sulfur atom.

Specific examples of the heteroaryl group include an imidazolyl group, a pyridyl group, a quinolyl group, a furyl group, a thienyl group, a benzoxazolyl group, a benzimidazolyl group, a benzthiazolyl group, a naphthothiazolyl group, a m-carbazolyl group, and an azepinyl group.

In a case where the heteroaryl group has a substituent, examples of the substituent include the same substituents as those mentioned as a monovalent substituent exemplified as R₉, R₁₀, R₁₁, R₁₂, and R₁₃ later.

Among these, from the viewpoint of synthetic suitability, the monovalent substituent is preferably a substituent selected from an aryl group, an alkoxy group, an alkoxycarbonyl group, a halogen atom, or an alkylamino group.

The number of heteroaryl groups having a substituent in R₁ to R₈ is preferably 1 to 8, more preferably 4 to 8, and still more preferably 8, that is, it is still more preferable that all the heteroaryl groups have a substituent.

The monovalent substituents included in the heteroaryl group may be the same or different from each other, but from the viewpoint of synthetic suitability, it is preferable that the monovalent substituents are the same.

In General Formula (1), R₉, R₁₀, R₁₁, R₁₂, and R₁₃ each independently represent a monovalent substituent, and n represents an integer of 0 to 4. In a case where n is an integer of 2 to 4, a plurality of R₉'s, R₁₀'s, R₁₁'s, R₁₂'s, and R₁₃'s may be the same or different from each other.

From the viewpoint of synthetic suitability, examples of the monovalent substituent include an alkyl group, an alkoxy group, a hydroxy group, a mercapto group, a halogen atom, a cyano group, a sulfo group, a carboxyl group, a nitro group, a hydroxamic acid group, a sulfino group, an acyl group, an alkoxycarbonyl group, an acyloxy group, an acylamino group, an alkoxycarbonylamino group, a sulfonylamino group, a sulfamoyl group, a carbamoyl group, an alkylthio group, a sulfonyl group, a sulfinyl group, and a ureido group.

The number of carbon atoms in the alkyl group is preferably 1 to 20, more preferably 1 to 10, and still more preferably 1 to 5. Examples of the alkyl group include methyl, ethyl, iso-propyl, tert-butyl, n-pentyl, cyclopropyl, and cyclopentyl.

The number of carbon atoms in the alkoxy group is preferably 1 to 20, more preferably 1 to 10, and still more preferably 1 to 5. Examples of the alkoxy group include methoxy, ethoxy, and butoxy.

Examples of the halogen atom include a fluorine atom and a chlorine atom.

The number of carbon atoms in the acyl group is preferably 1 to 20, more preferably 1 to 10, and still more preferably 1 to 5. Examples of the acyl group include acetyl, formyl, and pivaloyl.

The number of carbon atoms in the alkoxycarbonyl group is preferably 2 to 20, more preferably 2 to 10, and still more preferably 2 to 5. Examples of the alkoxycarbonyl group include methoxycarbonyl group and ethoxycarbonyl.

The number of carbon atoms in the acyloxy group is preferably 2 to 20, more preferably 2 to 10, and still more preferably 2 to 5. Examples of the acyloxy group include acetoxy.

The number of carbon atoms in the acylamino group is preferably 2 to 20, more preferably 2 to 10, and still more preferably 2 to 5. Examples of the acylamino group include acetylamino and isopropylamino.

The number of carbon atoms in the alkoxycarbonylamino group is preferably 2 to 20, more preferably 2 to 10, and still more preferably 2 to 6. Examples of the alkoxycarbonylamino group include methoxycarbonylamino.

The number of carbon atoms in the sulfonylamino group is preferably 1 to 20, more preferably 1 to 10, and still more preferably 1 to 5. Examples of the sulfonylamino group include methanesulfonylamino and isopropylsulfonylamino.

The number of carbon atoms in the sulfamoyl group is preferably 0 to 20, more preferably 0 to 10, and still more preferably 0 to 6. Examples of the sulfamoyl group include sulfamoyl, methylsulfamoyl, dimethylsulfamoyl, and phenylsulfamoyl.

The number of carbon atoms in the carbamoyl group is preferably 1 to 20, more preferably 1 to 10, and still more preferably 1 to 6. Examples of the carbamoyl group include carbamoyl, methylcarbamoyl, diethylcarbamoyl, and phenylcarbamoyl.

The number of carbon atoms in the alkylthio group is preferably 1 to 20, more preferably 1 to 10, and still more preferably 1 to 5. Examples of the alkylthio group include methylthio and ethylthio.

The number of carbon atoms in the sulfonyl group is preferably 1 to 20, more preferably 1 to 10, and still more preferably 1 to 6. Examples of the sulfonyl group include mesyl and tolyl.

The number of carbon atoms in the sulfinyl group is preferably 1 to 20, more preferably 1 to 10, and still more preferably 1 to 6. Examples of the sulfinyl group include methanesulfinyl and benzenesulfinyl.

The number of carbon atoms in the ureido group is preferably 1 to 20, more preferably 1 to 10, and still more preferably 1 to 6. Examples of the ureido group include ureido, methylureido, and phenylureido.

In General Formula (1), m is 1 or 2, and from the viewpoint of stability of the specific compound (1), m is preferably 2.

In General Formula (1), X⁻ represents a non-nucleophilic anion. That is, the specific compound (1) is a compound having a non-nucleophilic anion.

Here, the non-nucleophilicity of the anion means a property in which the anion does not perform a nucleophilic attack on the coloring agent, that is, a coloring agent mother nucleus of the specific compound (1) by heating. In a case where the coloring agent compound has a nucleophilic anion, the nucleophilic anion may attack the coloring agent mother nucleus and thus cause decomposition of the coloring agent depending on the heating conditions, which raises concerns about stability. On the other hand, in a case where the specific compound (1) has a non-nucleophilic anion, the attack of the anion on the coloring agent skeleton is suppressed, the decomposition of the specific compound (1) is suppressed, and the heat stability is maintained.

Examples of the non-nucleophilic anion include known non-nucleophilic anions described in paragraph of JP2007-310315A, the content of which is incorporated herein by reference.

Preferred examples of the non-nucleophilic anion include an imide anion (for example, a bis(sulfonyl)imide anion), a tris(sulfonyl)methide anion, a tetraarylborate anion, B—(CNₙ₁(ORa)₍₄₋ₙ₁₎ (Ra represents an alkyl group having 1 to 10 carbon atoms or an aryl group having 6 to 10 carbon atoms, and n1 represents 1 to 4), PFₙ₂R^{P}₍₆₋ₙ₂₎ (R^{P} represents a fluorinated alkyl group having 1 to 10 carbon atoms, and n2 represents an integer of 1 to 6), and BFₙ₃R^{P}_{(4 n3)} (R^{P} represents a fluorinated alkyl group having 1 to 10 carbon atoms, and n3 represents an integer of 1 to 4).

From the viewpoint that the stability of the specific compound (1) is more favorable, preferred examples of the non-nucleophilic anion include a bis(alkylsulfonyl)amide anion, a tris(alkylsulfonyl)methide anion, BF₄⁻, PF₆⁻, SbF₆⁻, a perchlorate anion, cyclopentadienide, B(CN)₄⁻, B(Ph)₄⁻, and B(C₆F₅)₄⁻; more preferred examples thereof include a bis(alkylsulfonyl)amide anion, a tris(alkylsulfonyl)methide anion, BF₄⁻, PF₆⁻, SbF₆⁻, a perchlorate anion, B(CN)₄^{″}, B(Ph)₄^{″}, and B(C₆F₅)₄⁻; and still more preferred examples thereof include a bis(alkylsulfonyl)amide anion, a tris(alkylsulfonyl)methide anion, SbF₆-, a perchlorate anion, B(Ph)₄⁻, B(CN)₄⁻, and B(C₆F₅)₄⁻.

A position of a cation of an immonium compound skeleton in the specific compound (1) is not particularly limited, and it can take various forms in consideration of a conjugated compound. Among these, it is preferable that a portion of any nitrogen atom in the diimmonium skeleton is a cation.

From the viewpoint of contribution rate of the conjugated structure that the specific compound (1) can be formed, for example, in a case where m is 2, N⁺ can be a cationic moiety as in a structure represented by Formula (1-1), and in a case where m is 1, N⁺ can be a cationic moiety as in a structure represented by Formula (1-2). However, as described above, the position of the cationic moiety which contributes to the interaction with the anion in the diimmonium skeleton is not limited to the following examples.

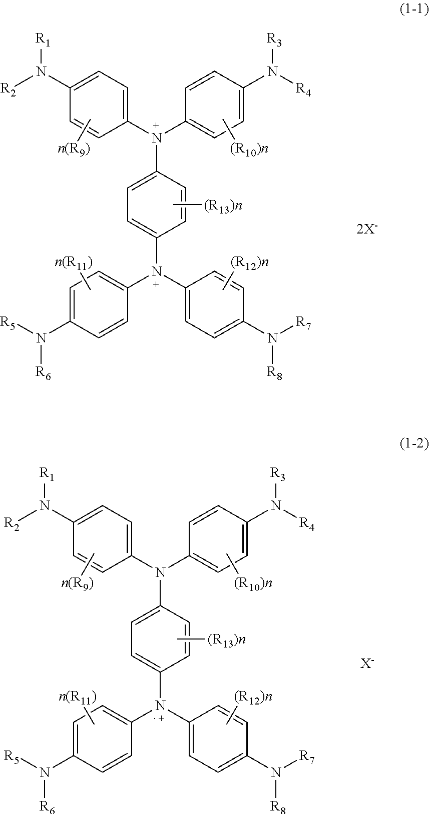

In Formulae (1-1) and (1-2), R₁ to R₈, R₉ to R₁₃, n, and X⁻ have the same meanings as R₁ to R₈, R₉ to R₁₃, n, and X⁻ in General Formula (1), and preferred examples thereof are also the same.

A second embodiment of the coloring agent composition according to the present disclosure (hereinafter, also simply referred to as “composition (2)”) contains a compound represented by General Formula (2) (hereinafter, also simply referred to as “specific compound (2)”).

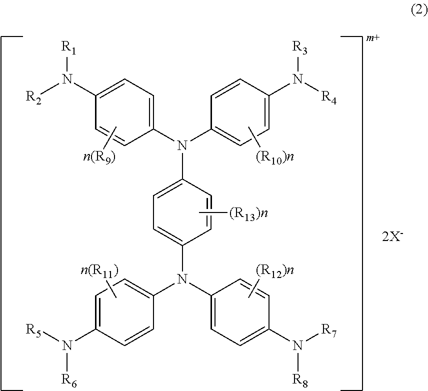

In General Formula (2), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where two or more of R₁, R₂, R₃, R₄, R₅, R₆, R₇, and R₈ may be linked to each other to form a ring.

In General Formula (2), R₉, R₁₀, R₁₁, R₁₂, and R₁₃ each independently represent a substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other, and

X⁻ represents a non-nucleophilic anion.

The second embodiment of the coloring agent composition according to the present disclosure contains the specific compound (2) instead of the specific compound (1) in the first embodiment of the coloring agent composition according to the present disclosure. The specific compound (2) is a compound in which m in the specific compound (1) is 2.

R₁, R₂, R₃, R₄, R₅, R₆, R₇, R₈, R₉, R₁₀, R₁₁, R₁₂, R₁₃, n, and X⁻ in General Formula (2) have the same meanings as those in General Formula (1), and preferred examples thereof are also the same.

The specific compound (1) and the specific compound (2) can be synthesized, for example, according to the following scheme with an intermediate X-1 to be synthesized. The following compound A-1 is a compound included in the specific compound (1), and the following compound B-1 is a compound included in the specific compound (2).

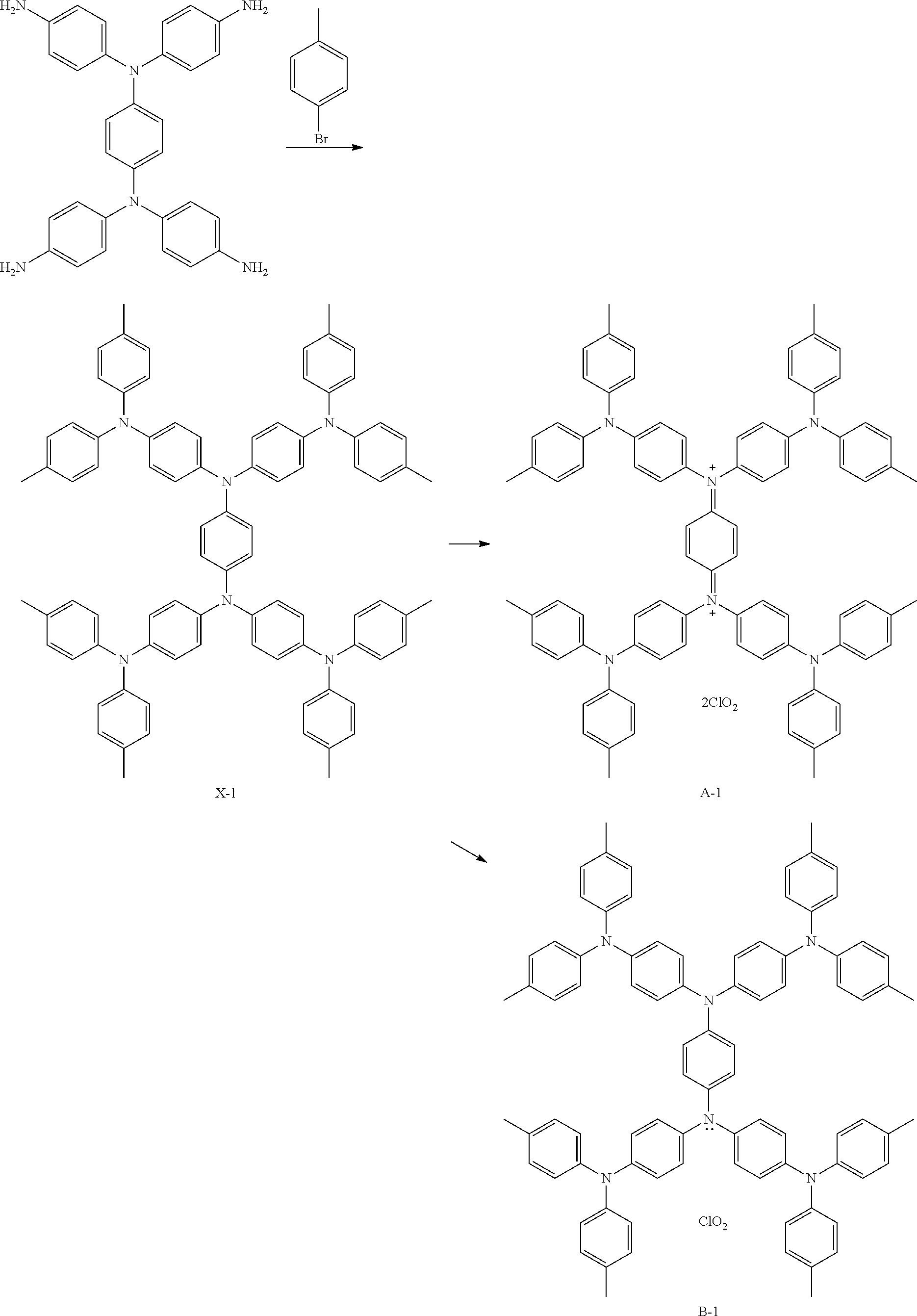

In the following, both or one of the specific compound (1) and the specific compound (2) according to the present disclosure may be collectively referred to as “specific compound according to the present disclosure”. In addition, both or one of the composition (1) and the composition (2) according to the present disclosure may be collectively referred to as “composition according to the present disclosure”.

The specific compound according to the present disclosure preferably has a maximal absorption wavelength in an infrared region of a wavelength of 700 nm to 2,500 nm, and more preferably has a maximal absorption wavelength in a near infrared region of a wavelength of 1,150 nm to 2,000 nm.

The near infrared absorbability of the specific compound is evaluated as follows.

The specific compound is diluted with chloroform to a concentration of 3.0×10⁻ mol/L, and an absorbance of the obtained sample solution is measured with a spectrophotometer (UV-3600 Plus, manufactured by Shimadzu Corporation) using a 1 mm quartz cell. A maximal absorption wavelength (Amax) is measured from an absorption spectrum of the sample solution.

The reason why the composition (1) and the composition (2), containing the specific compound according to the present disclosure, have absorption in a long-wavelength infrared region and are thermally stable is presumed as follows.

The specific compound according to the present disclosure has a ring structure such as an aryl group or has a heteroaryl group at a nitrogen atom of a diimmonium skeleton, and thus the compound itself exhibits favorable near infrared absorbing property. Here, since the compound has a non-nucleophilic anion as a counterion of the coloring agent skeleton including the diimmonium skeleton, a nucleophilic attack caused by the counterion is suppressed even under heating conditions, so that heat stability of the compound is improved.

Furthermore, since the aryl group or the like, which is bonded to the nitrogen atom of the diimmonium skeleton, has a substituent or the nitrogen atom of the diimmonium skeleton has a heteroaryl group, affinity with a solvent, a resin, and the like, which are contained in the composition, is improved, and the diimmonium skeletons are less likely to approach each other, so that uniform dispersibility of the specific compound in the composition is improved in a case of being used as a coloring agent composition. In addition, it is considered that, since the aryl group or the like, which is bonded to the nitrogen atom of the diimmonium skeleton, has the substituent having a σp value on Hammett's rule of −0.5 or more, further oxidation reaction is less likely to occur, and the compound is capable of being synthesized as a divalent and monovalent cation and is less likely to cause thermal oxidative decomposition.

That is, it is considered that the specific compound in the present disclosure can solve the above-described problems in that the coloring agent mother nucleus having a diimmonium skeleton has the non-nucleophilic anion, and the diimmonium skeleton has an aryl group having the substituent having a σp value on Hammett's rule of −0.5 or more or has a heteroaryl group.

Specific examples of the specific compound in the present disclosure are shown below, but it goes without saying that the specific compound according to the present disclosure is not limited to the following examples.

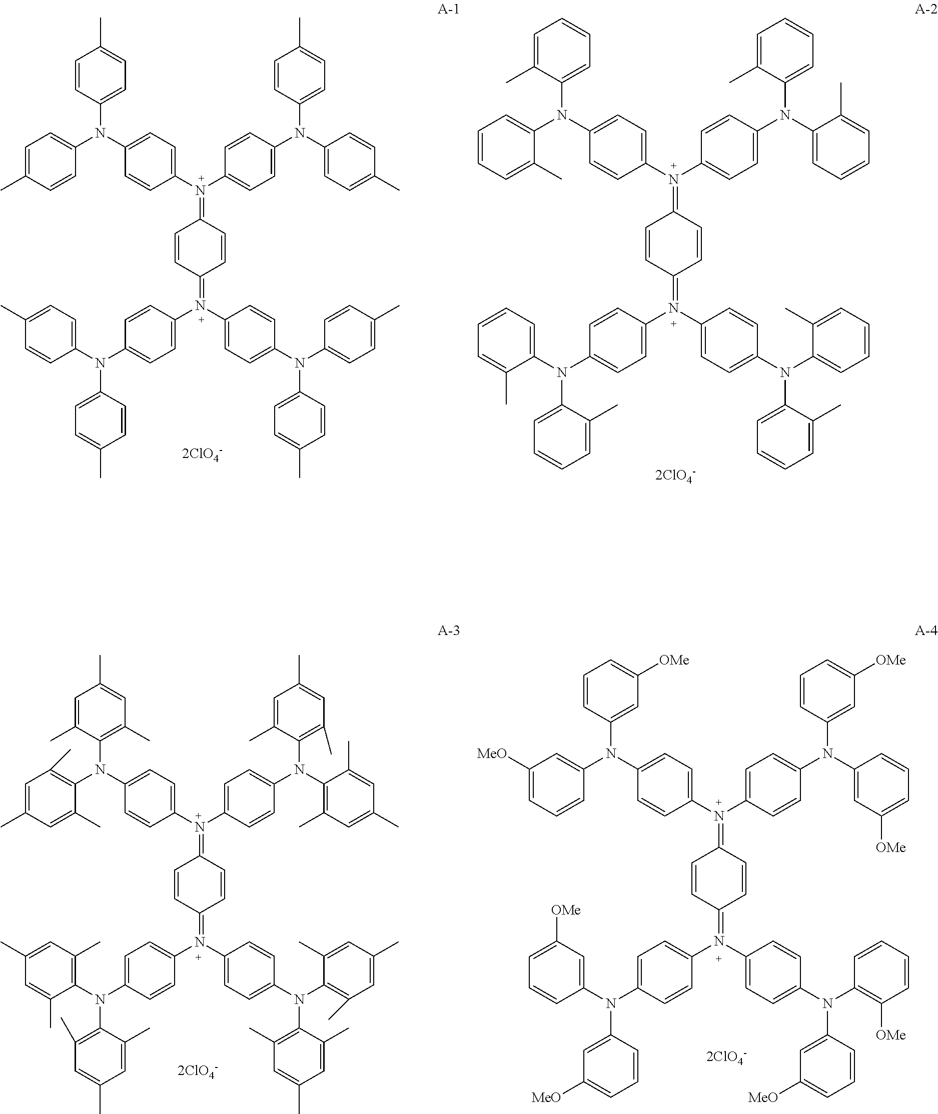

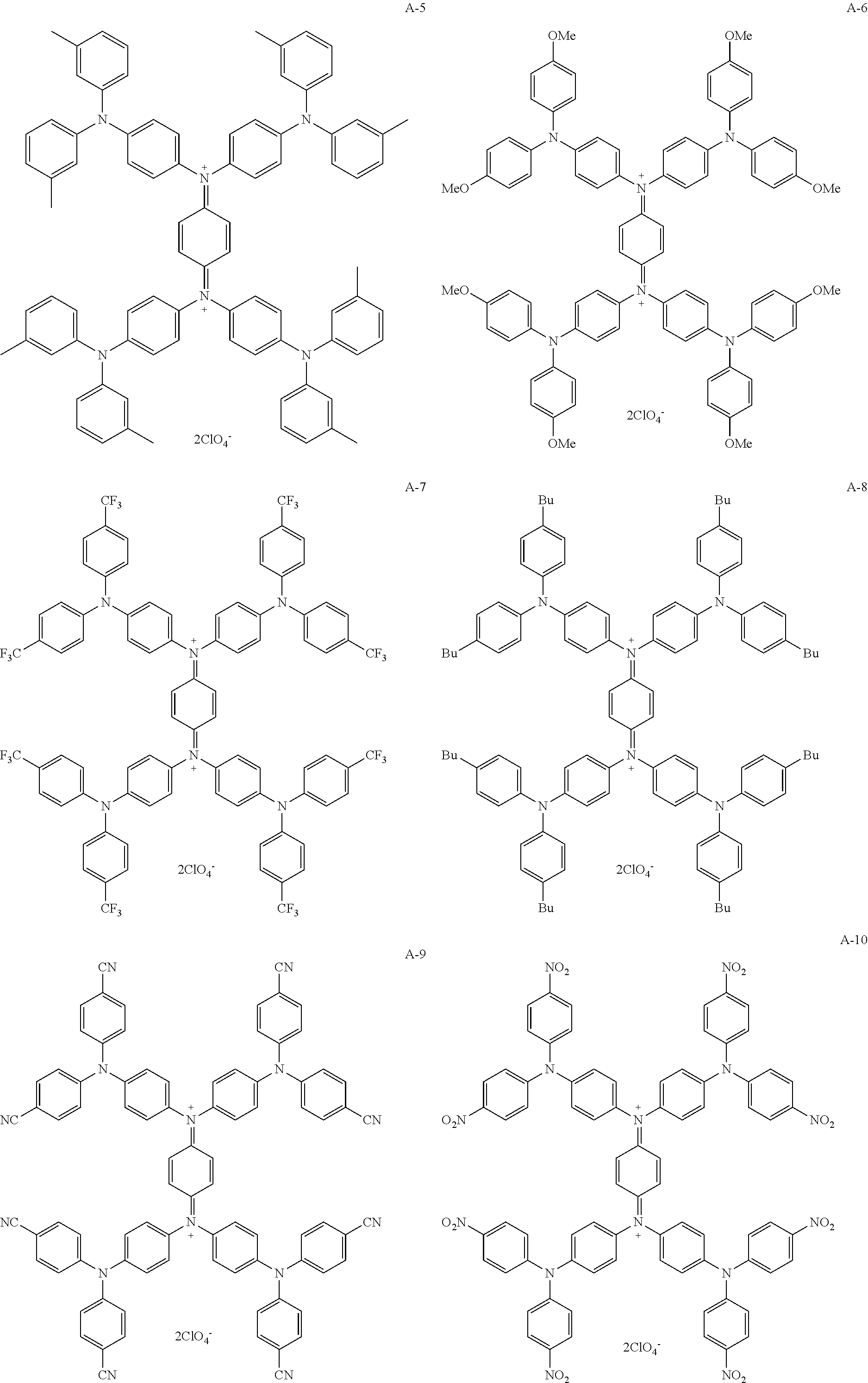

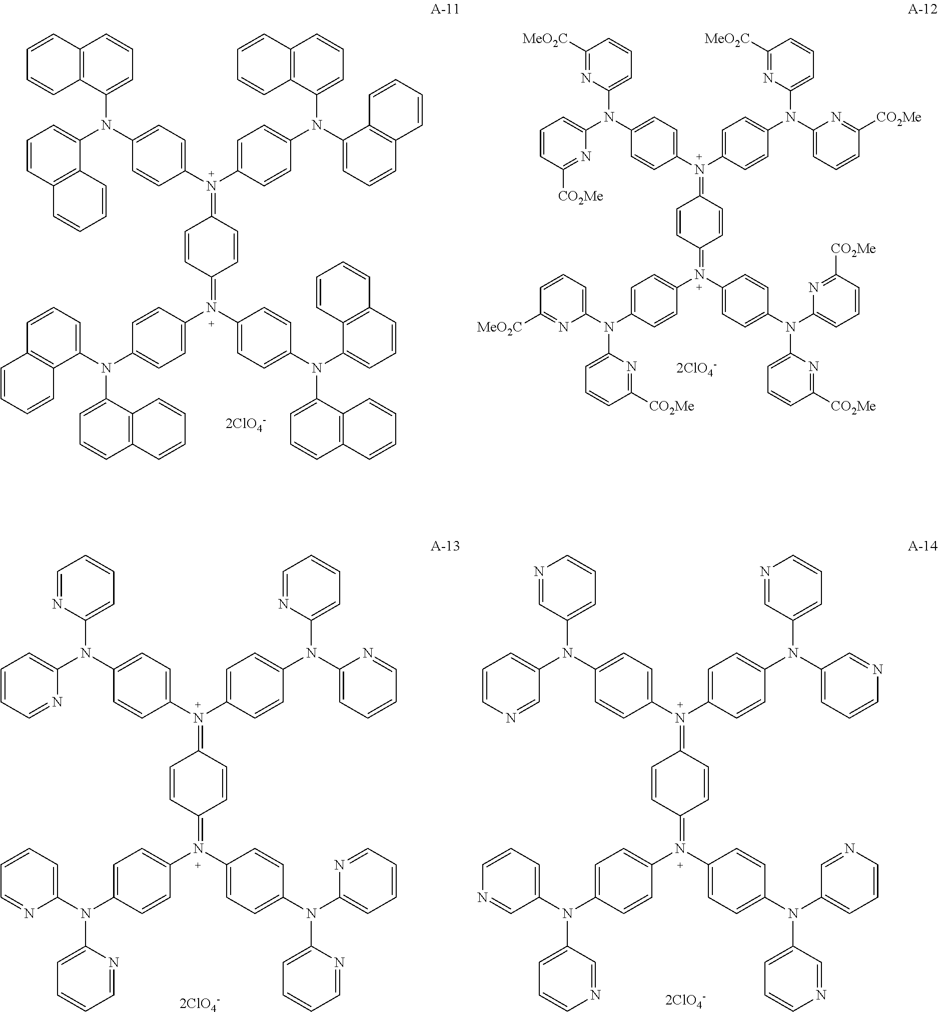

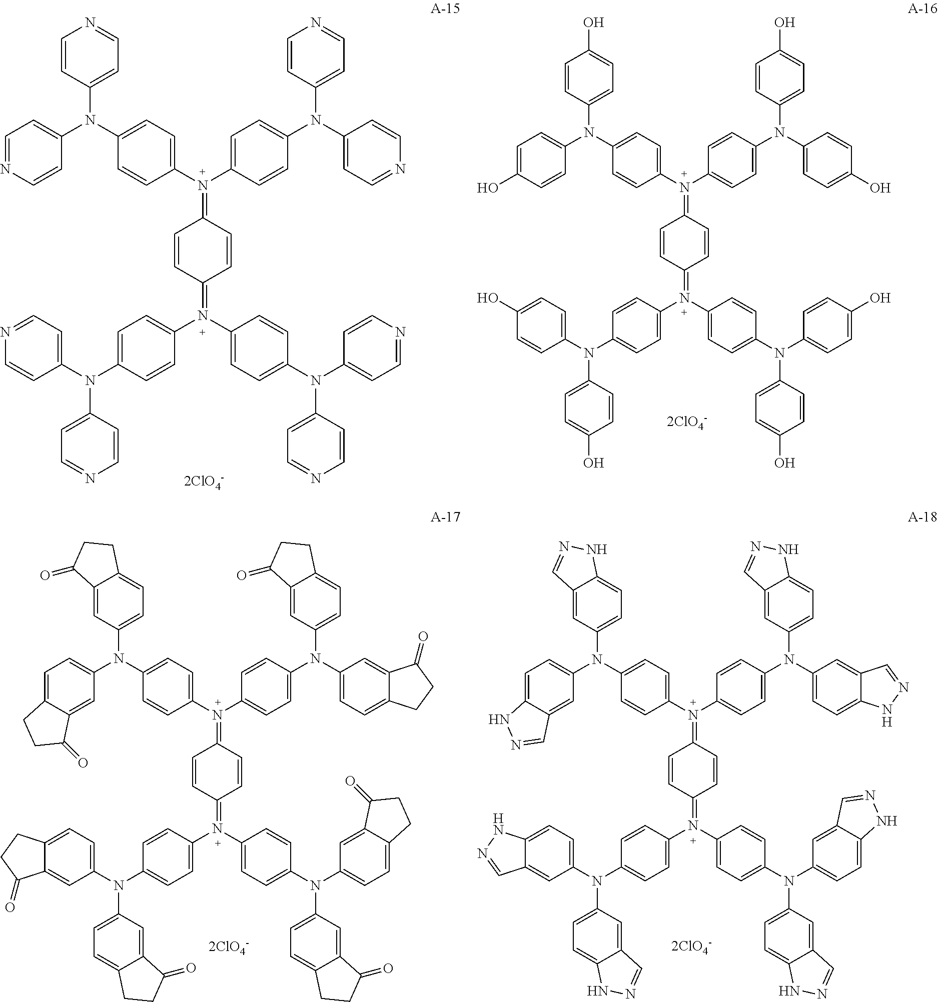

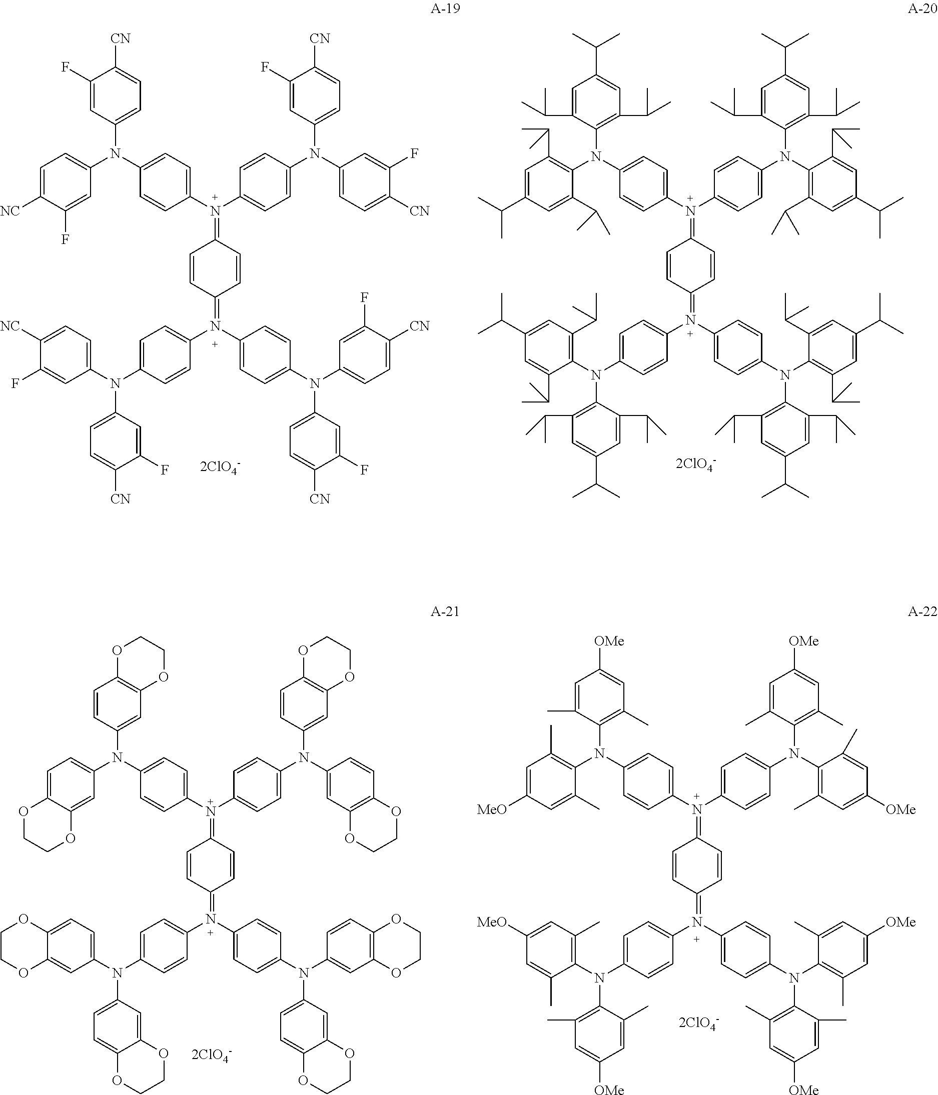

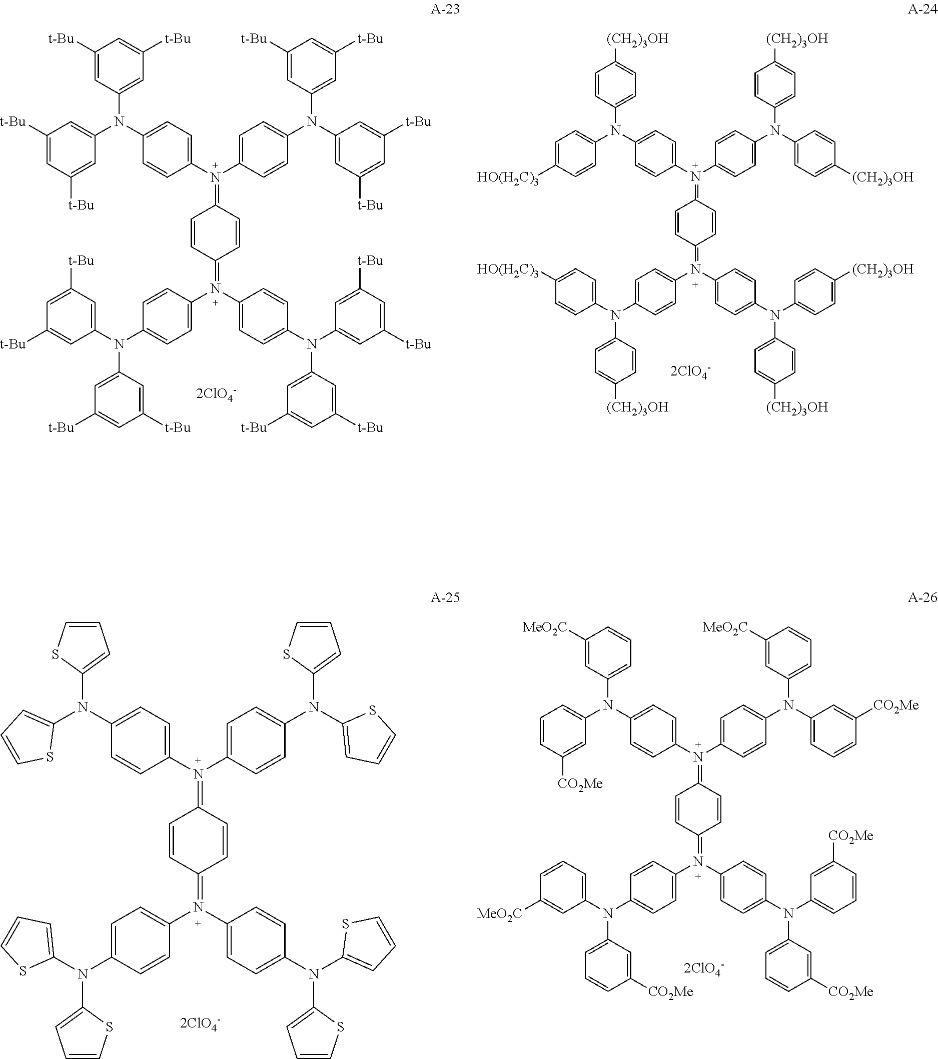

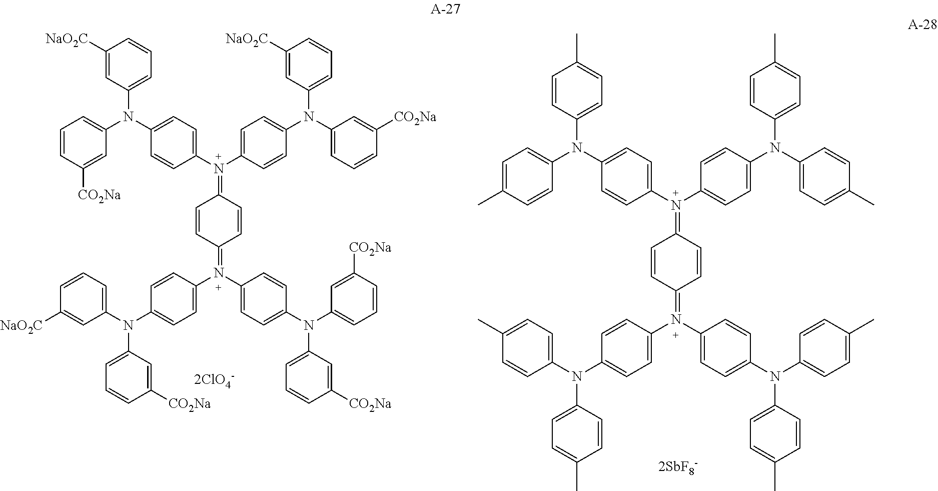

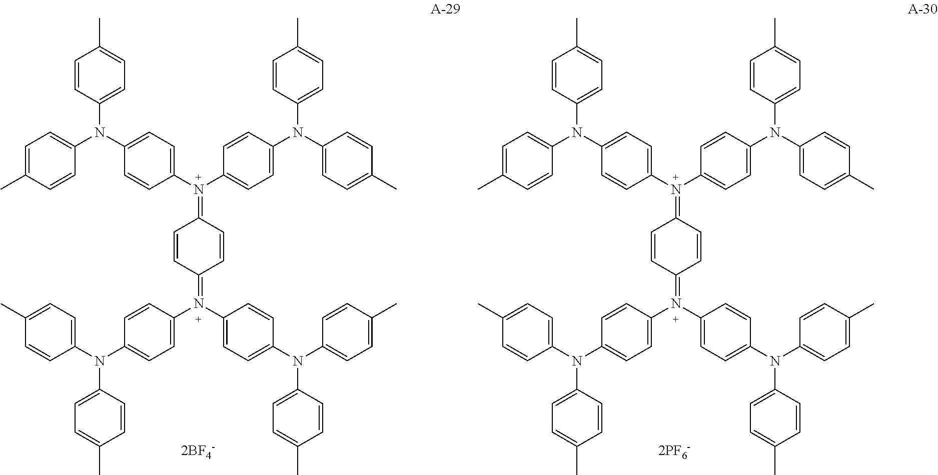

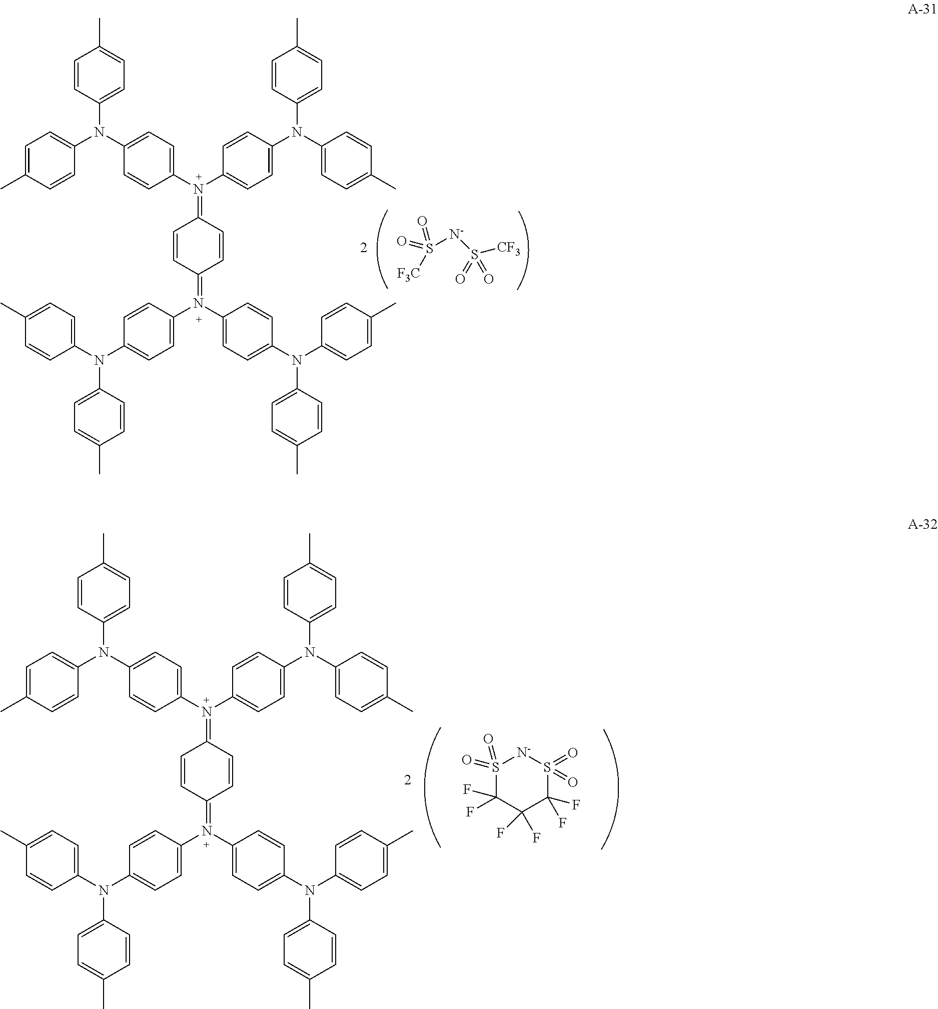

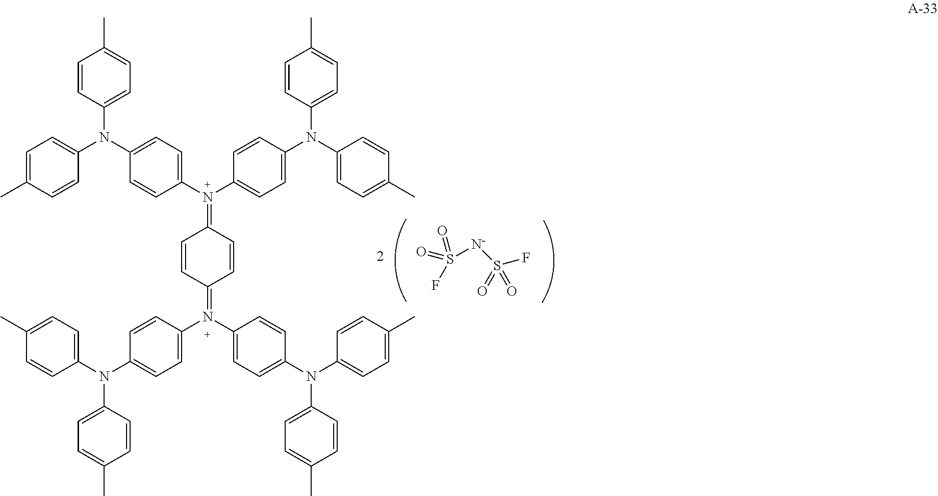

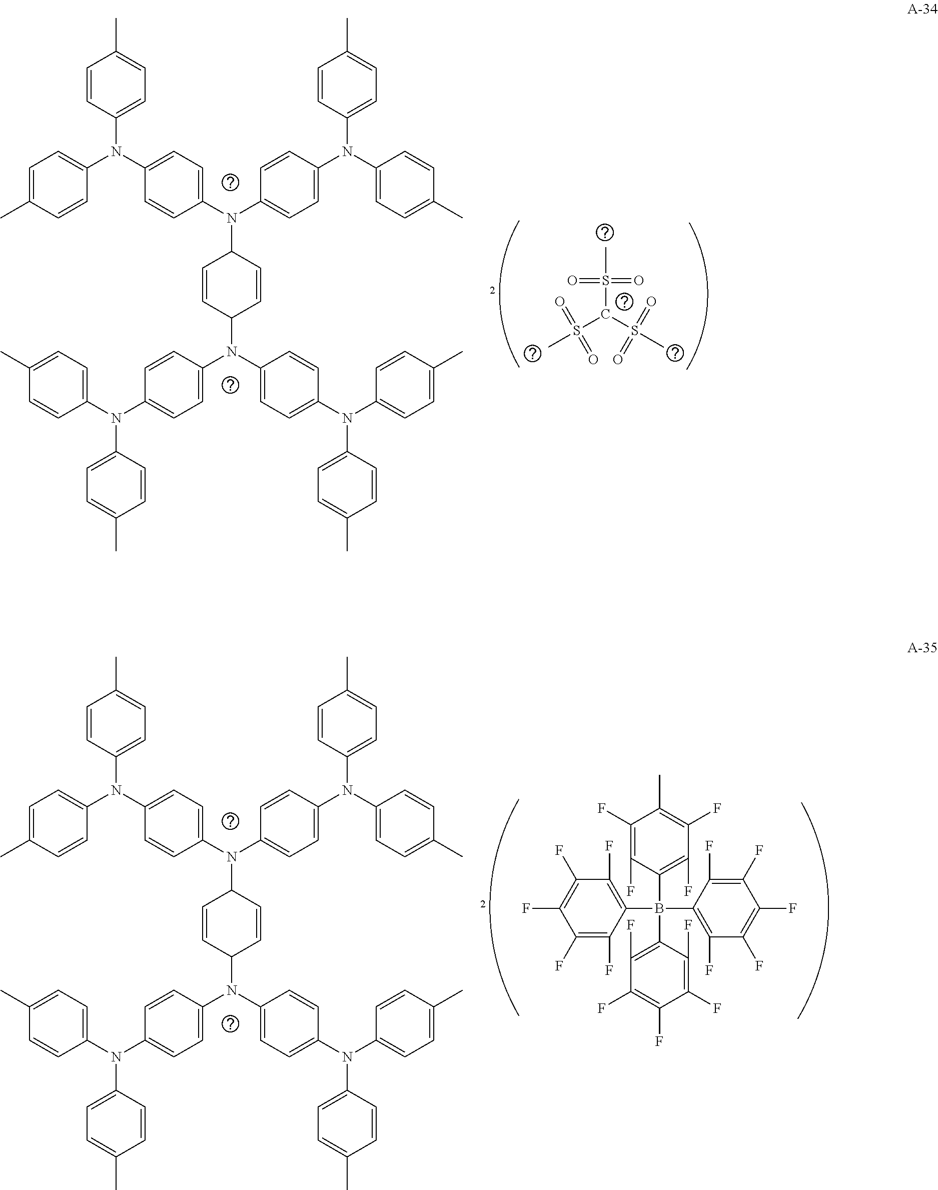

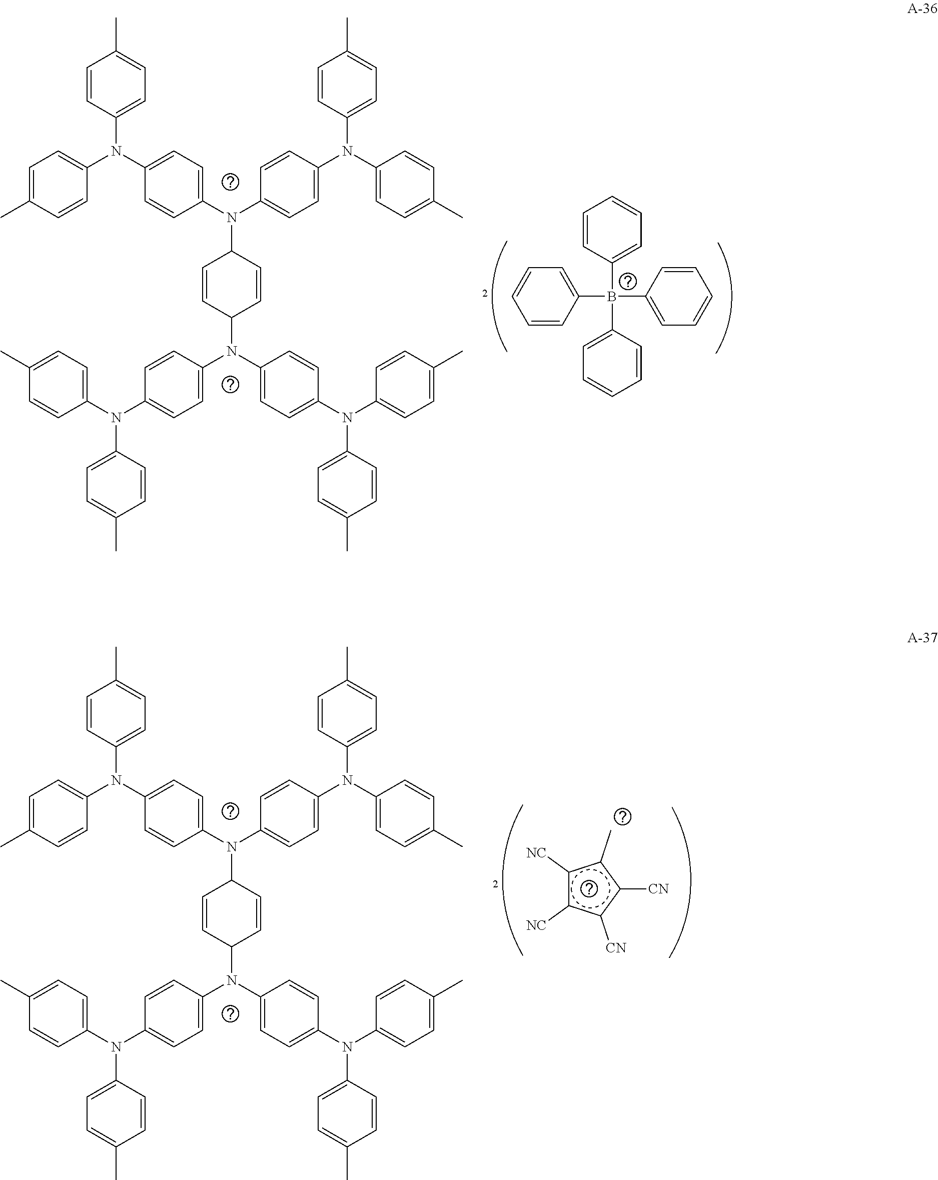

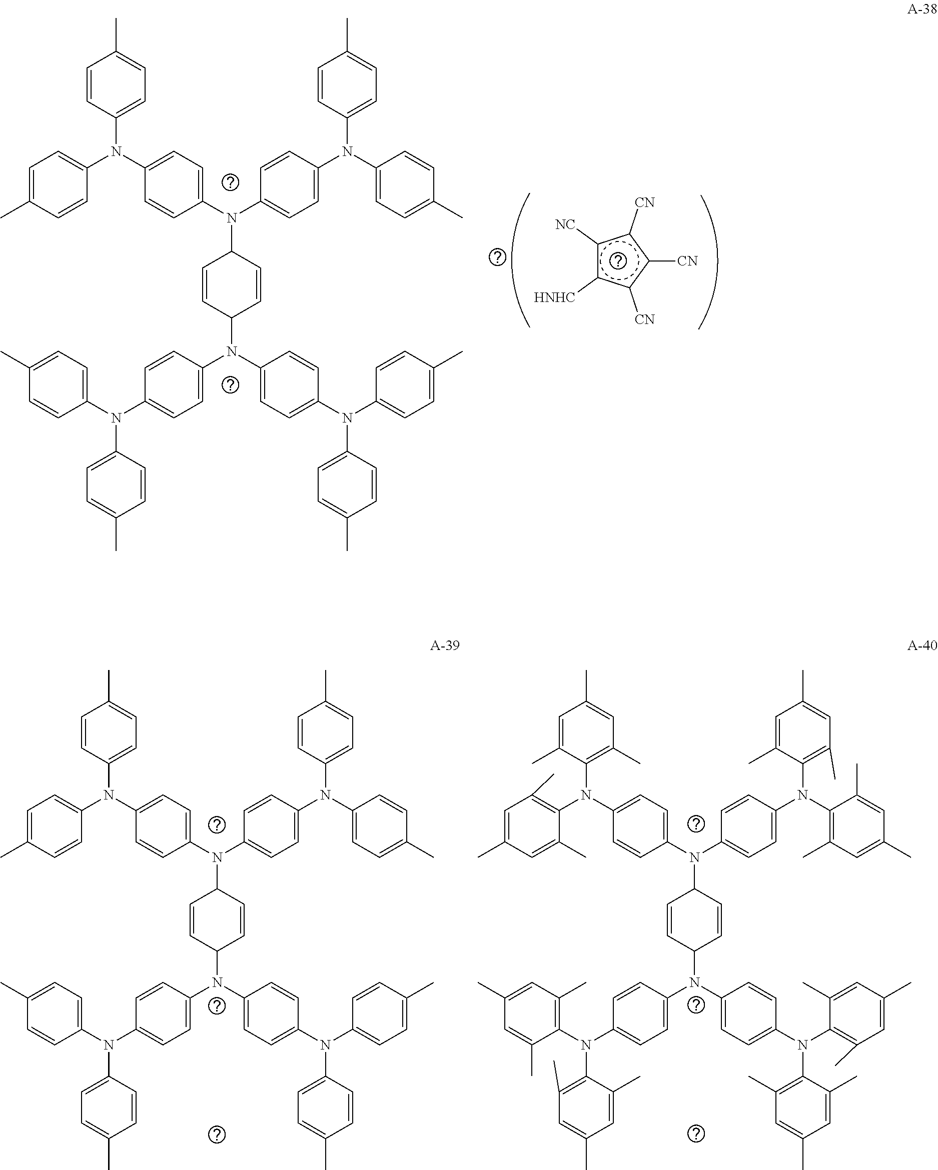

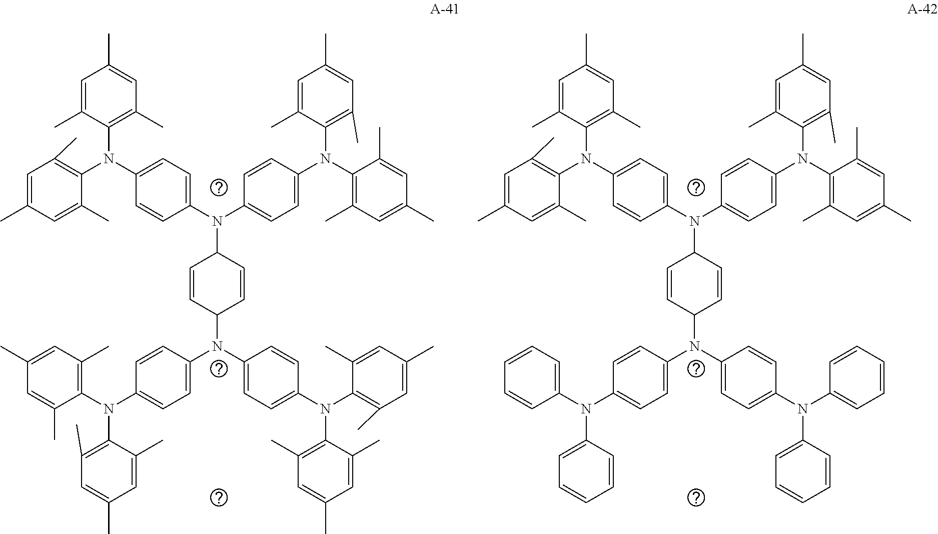

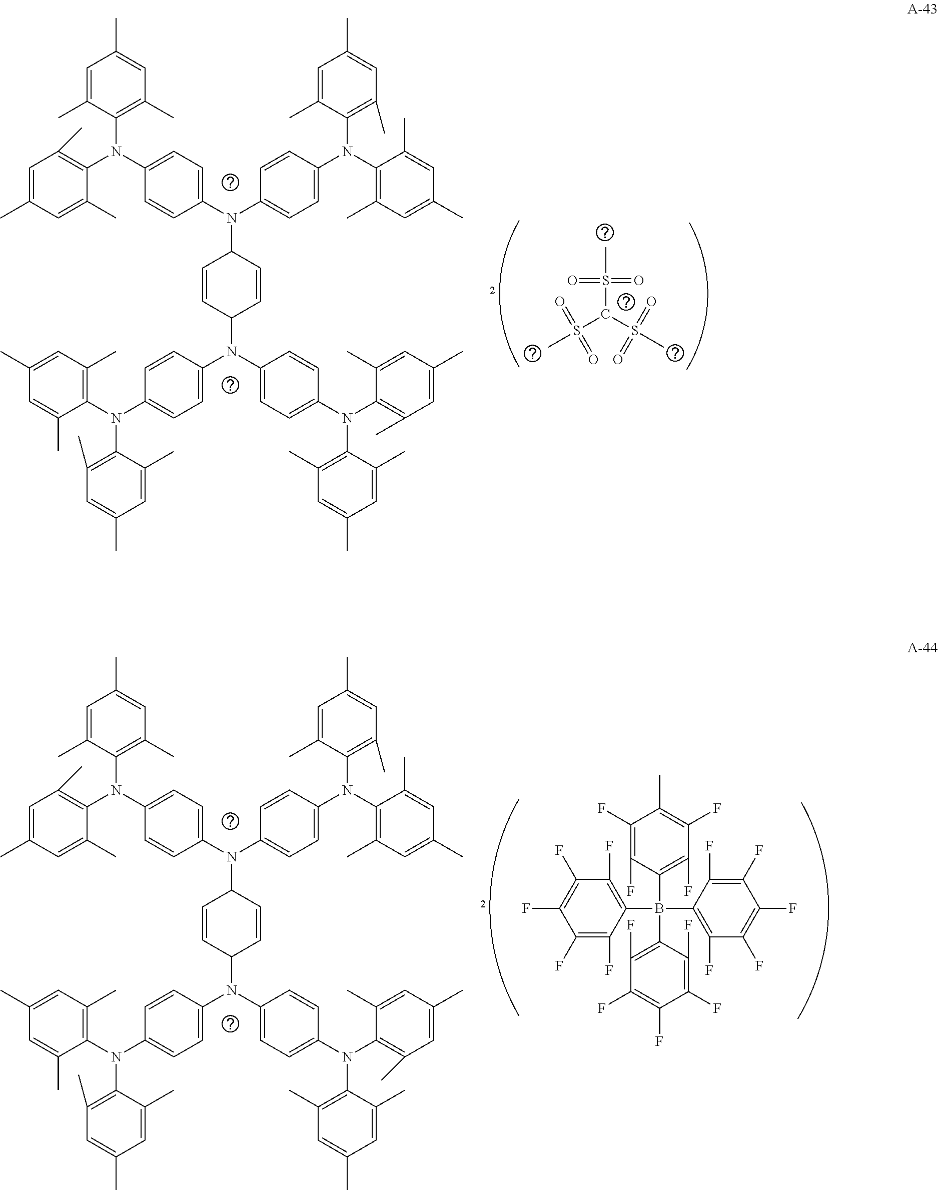

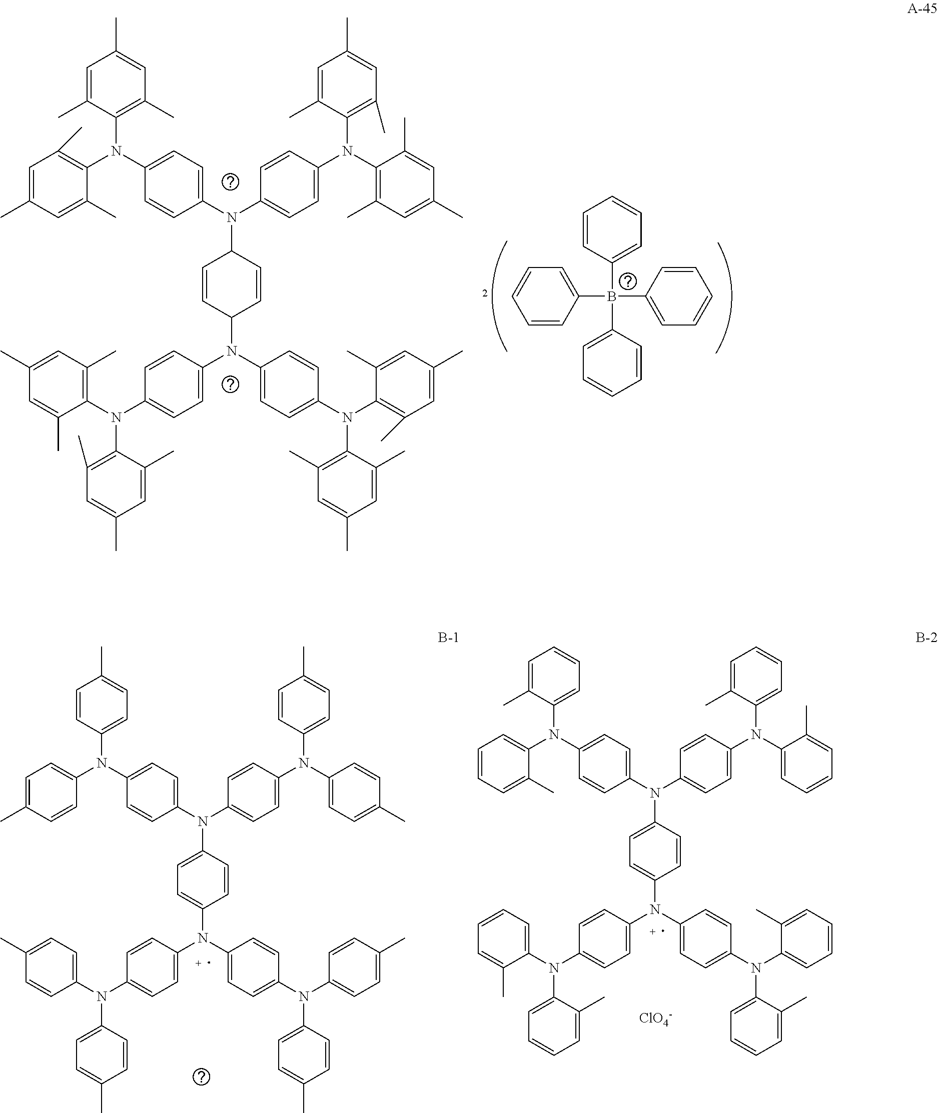

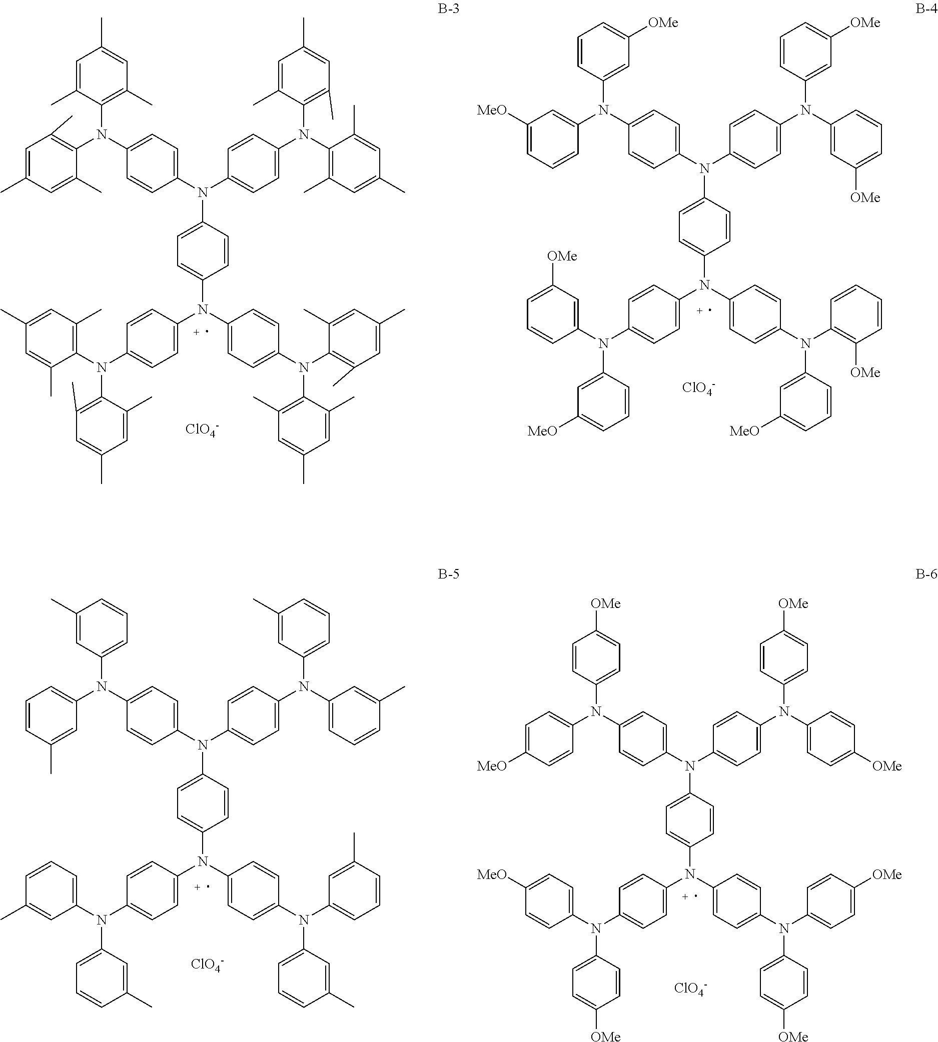

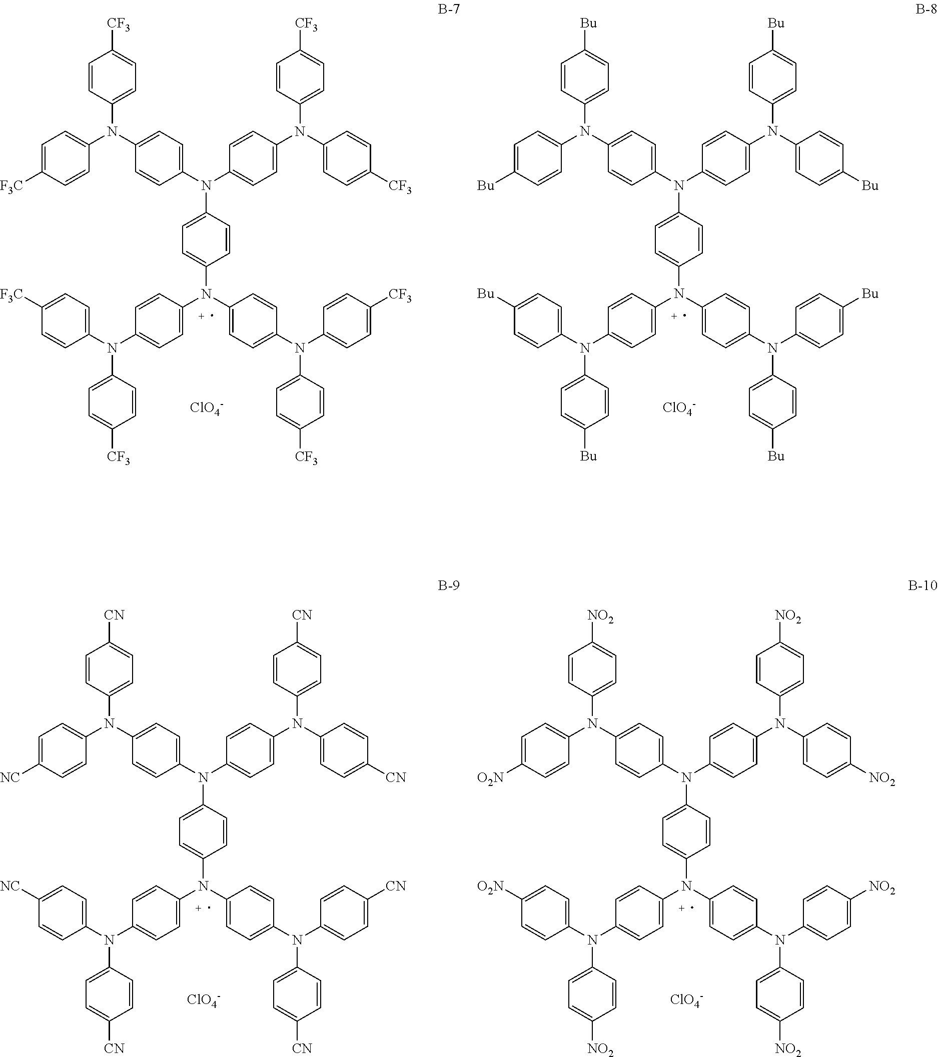

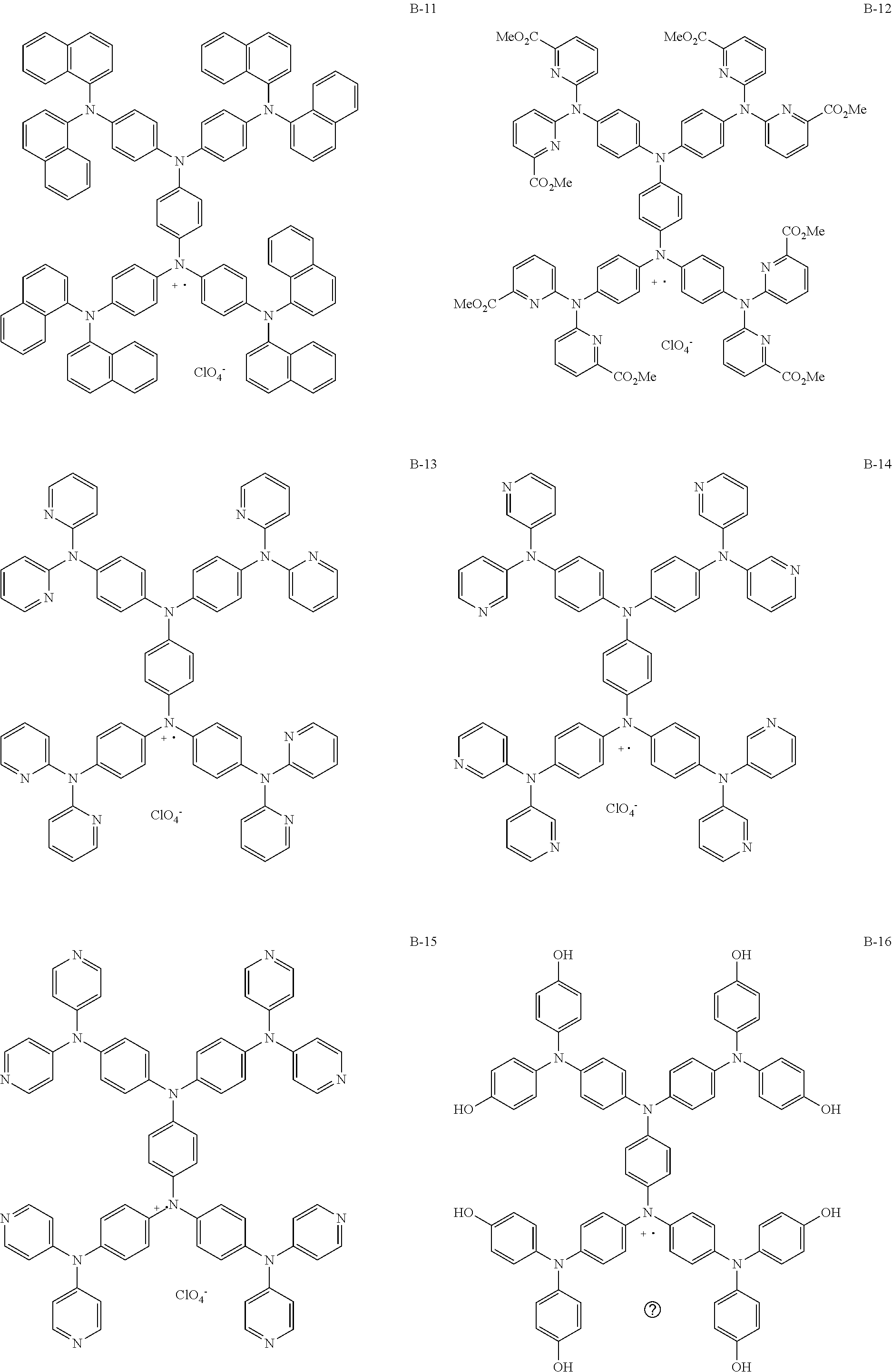

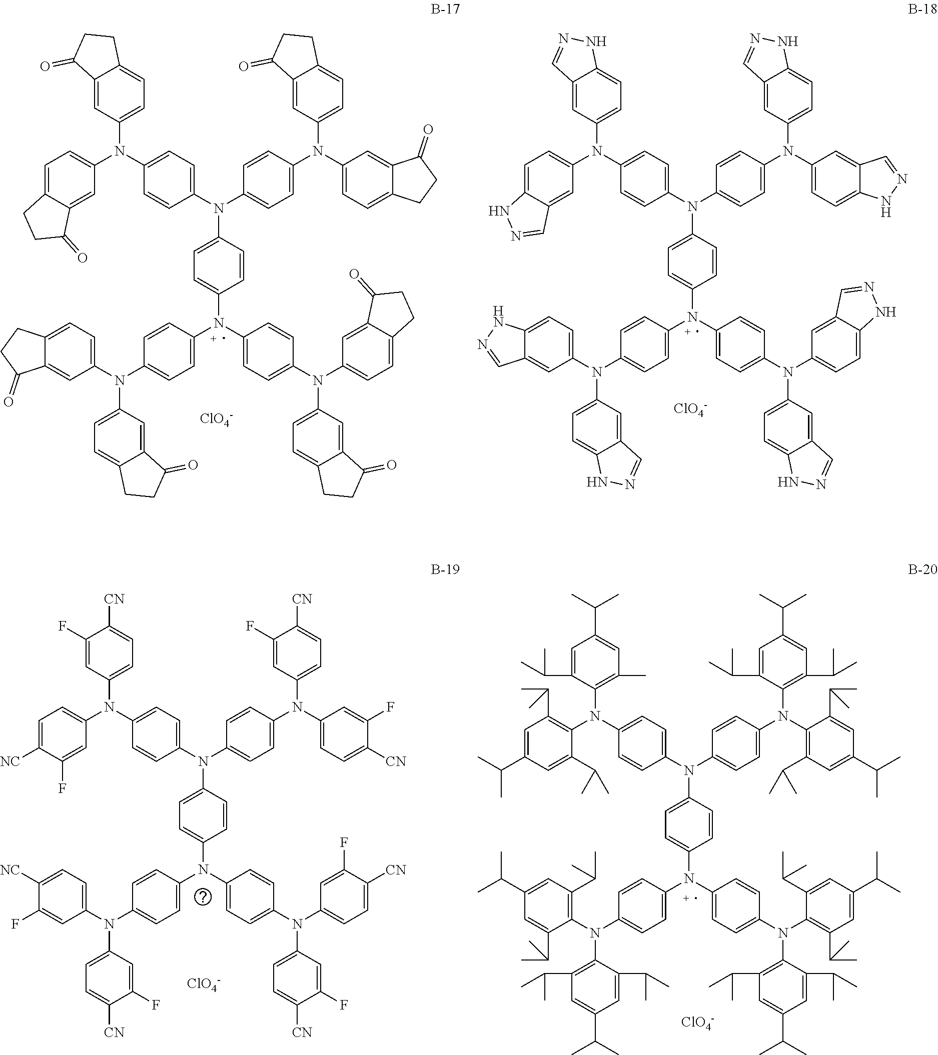

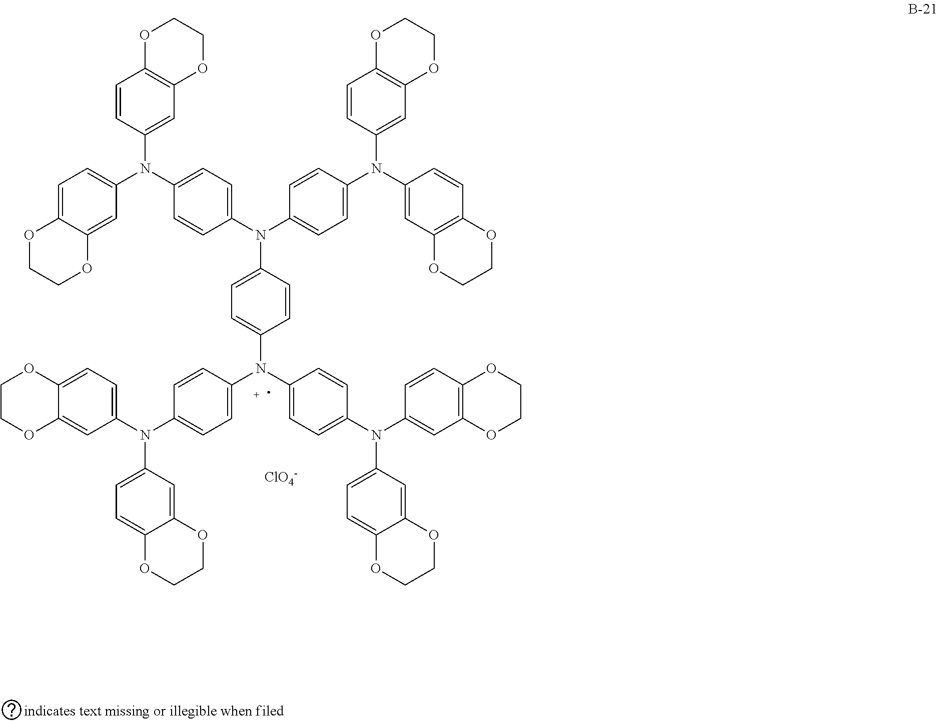

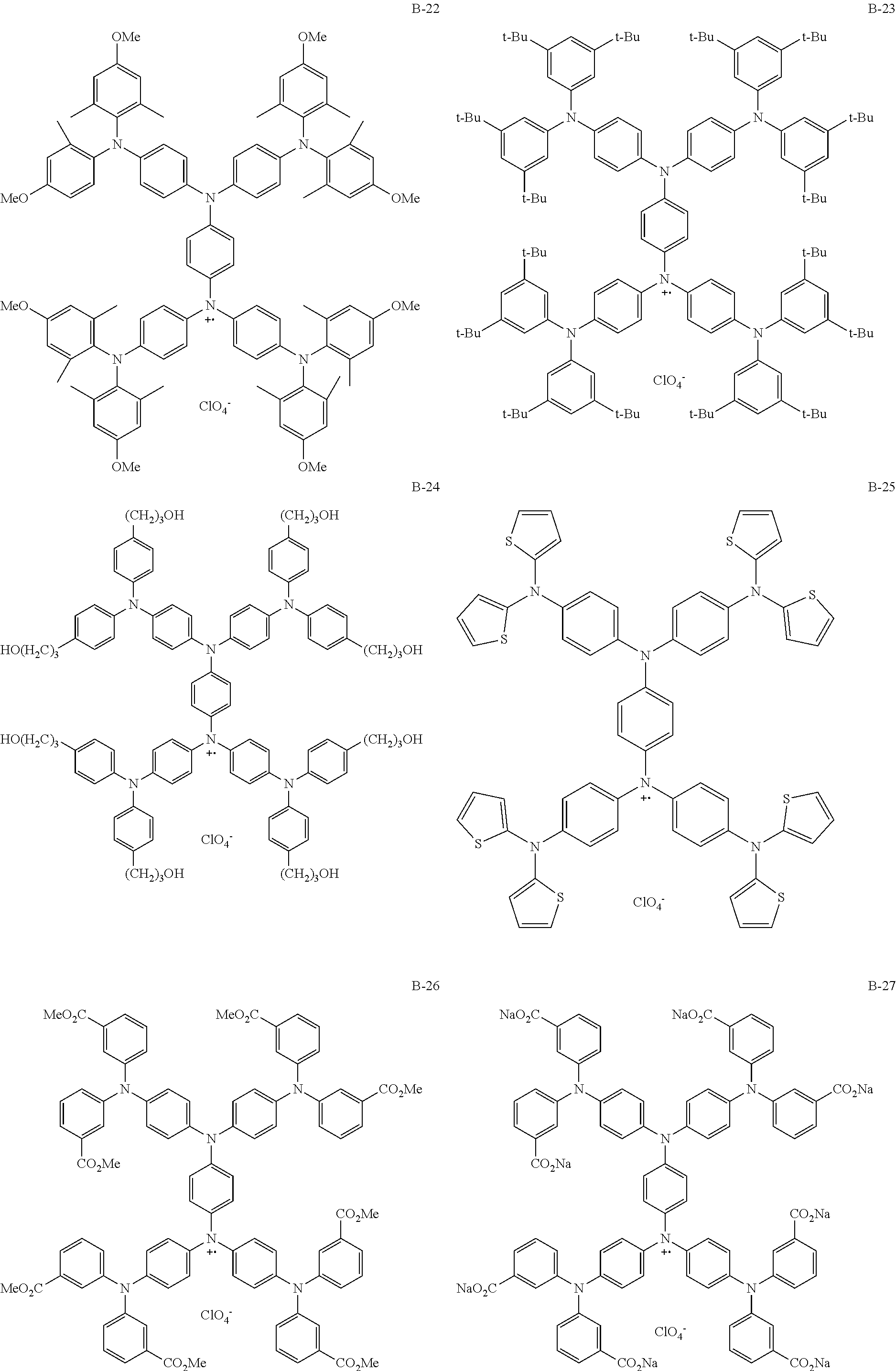

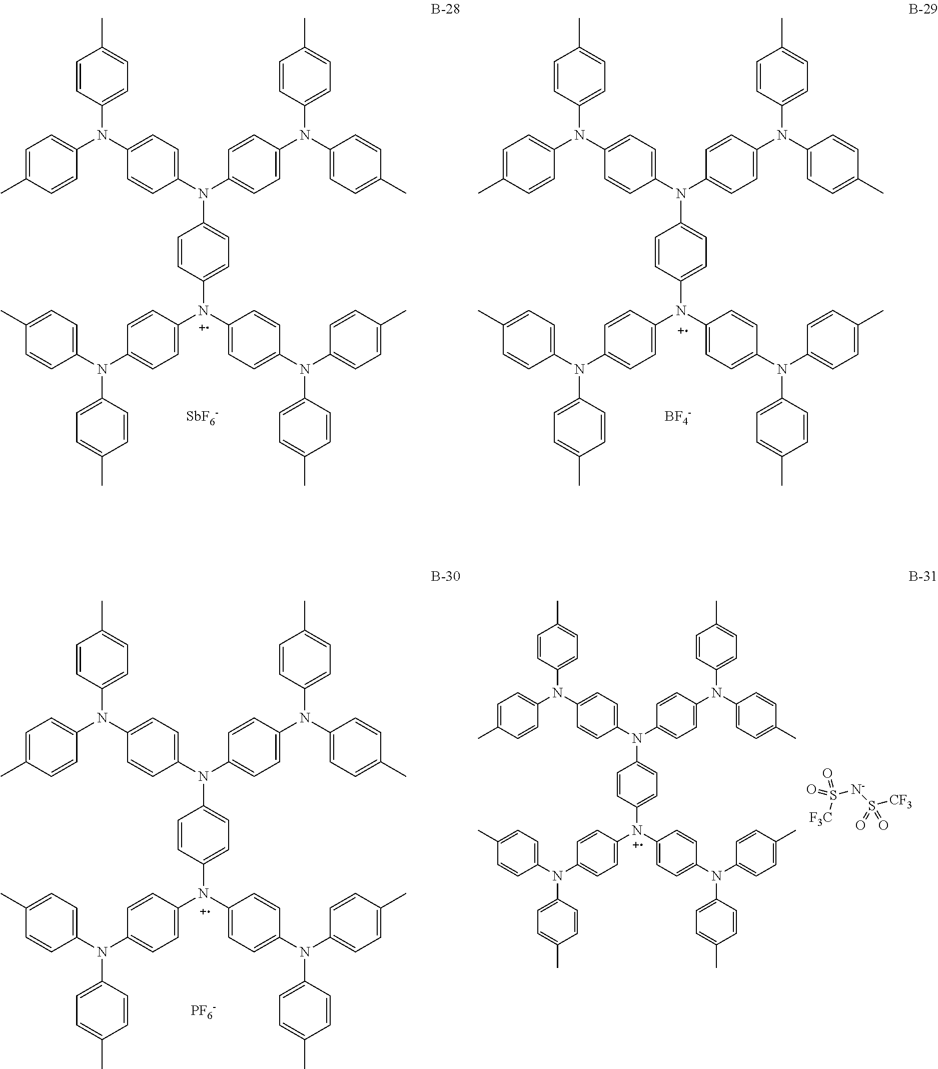

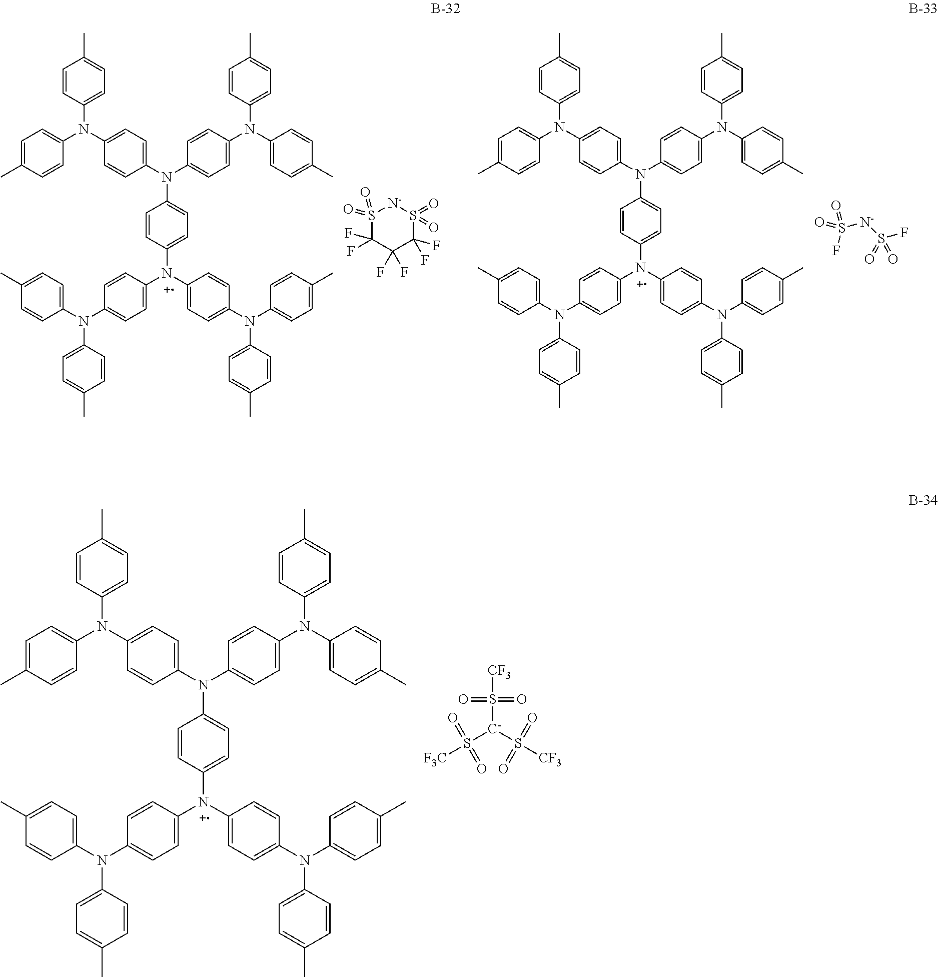

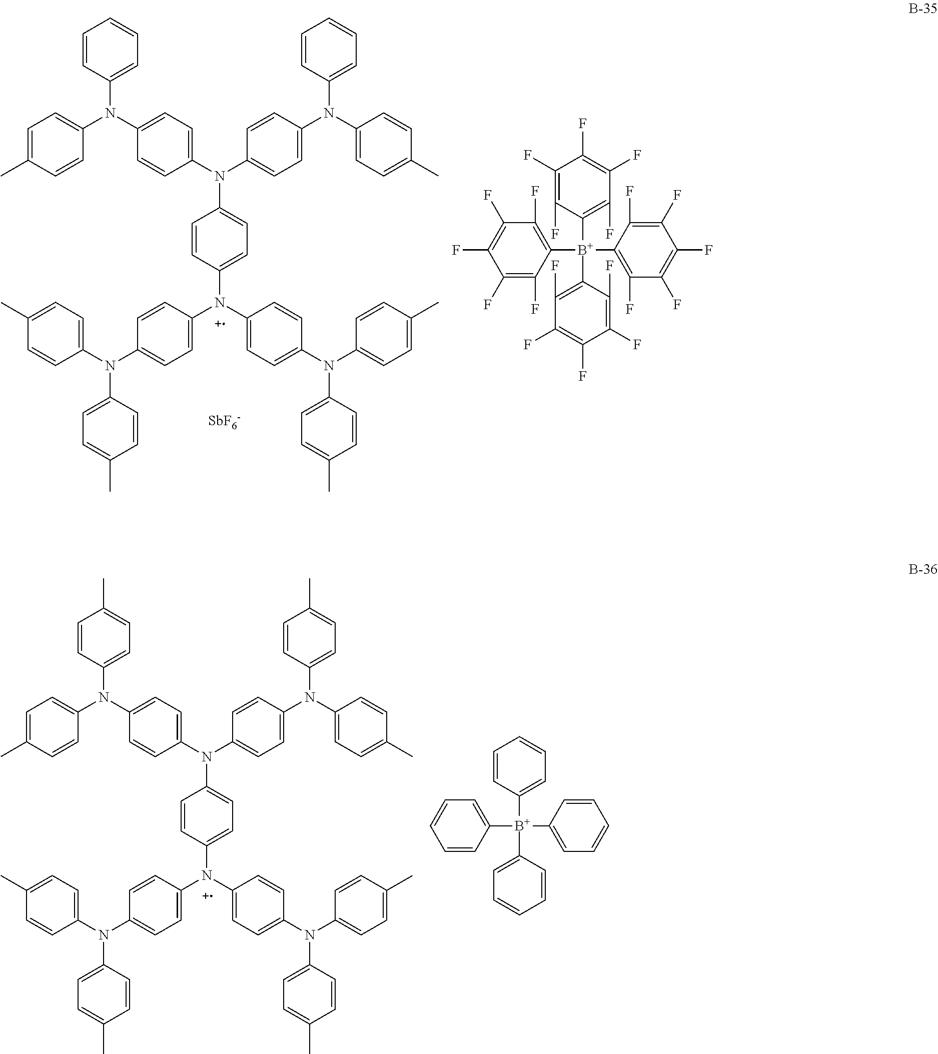

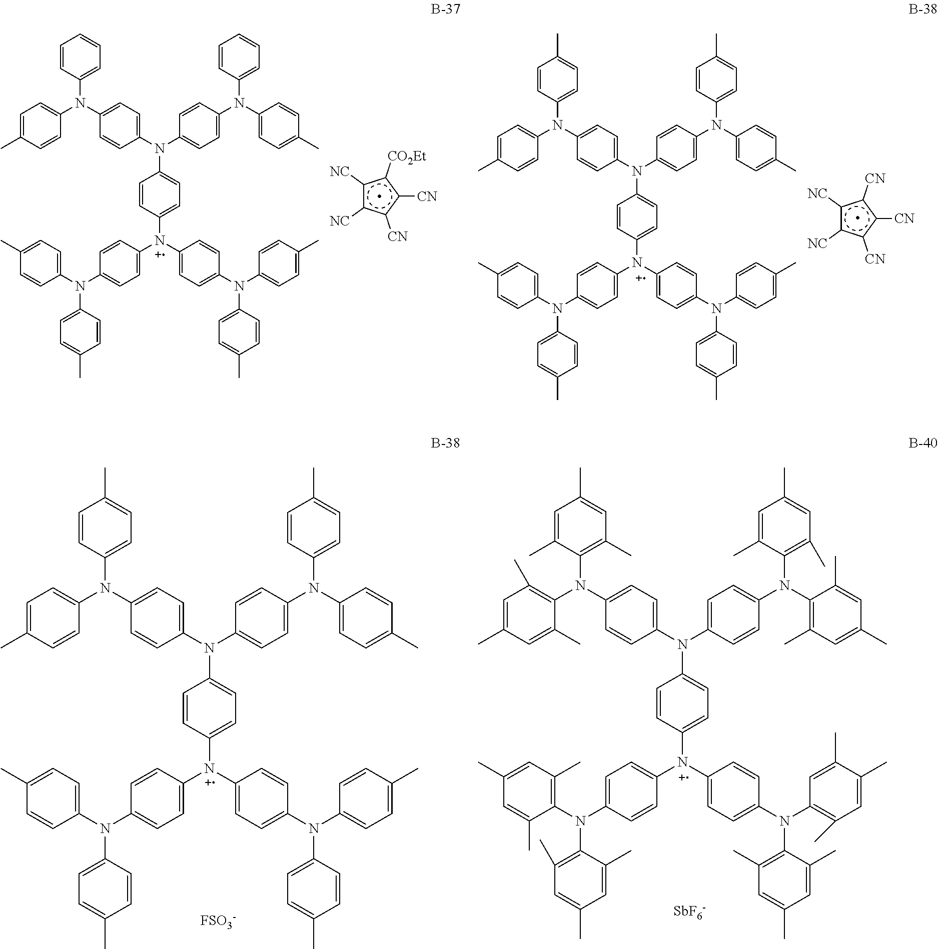

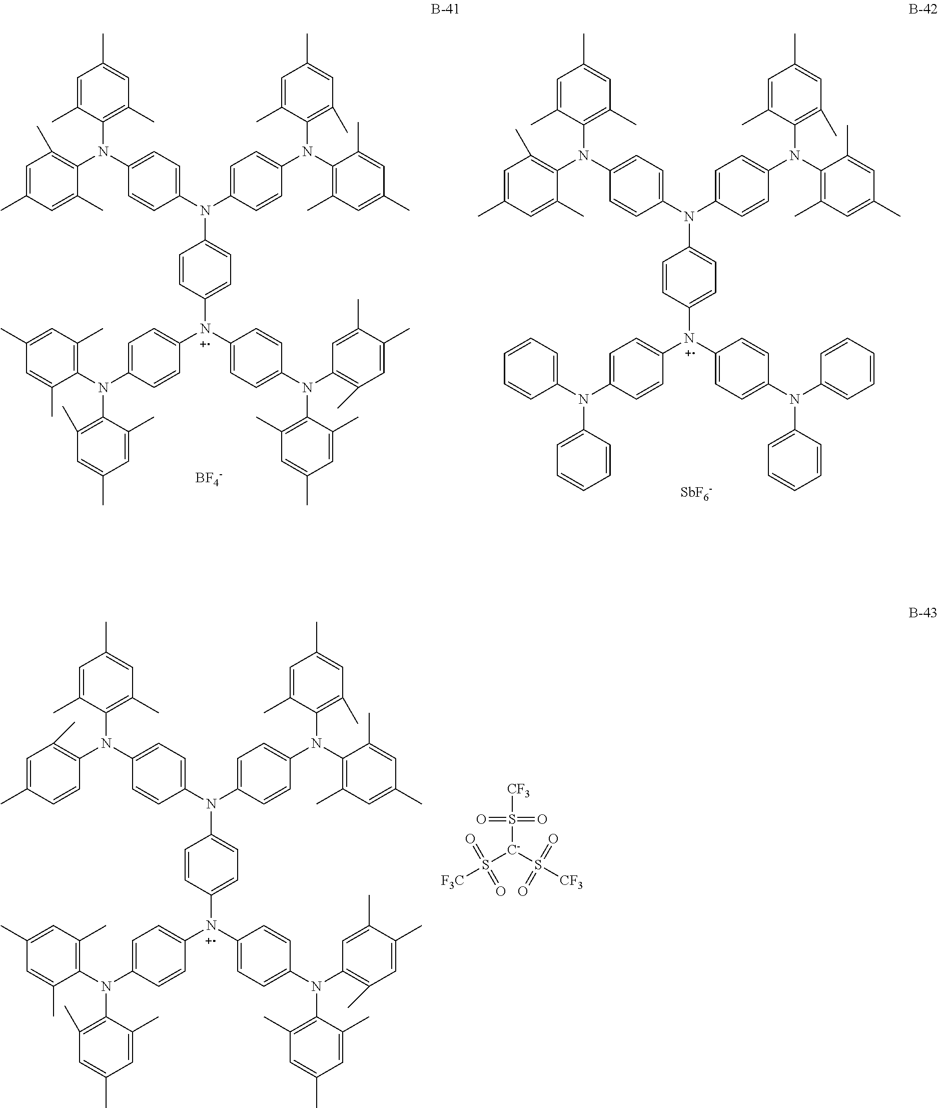

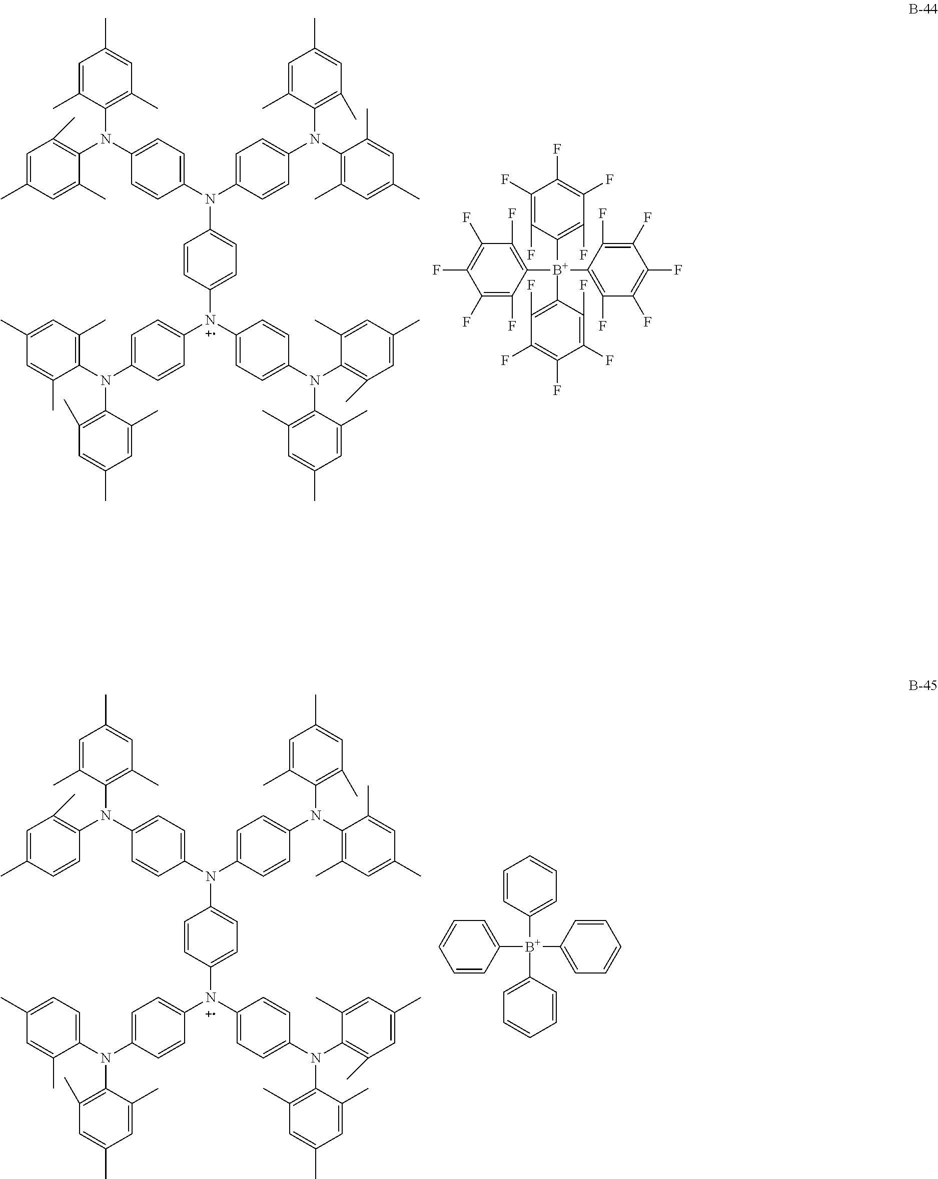

Since the composition according to the present disclosure contains at least one specific compound having the above-described properties, the composition has excellent near infrared absorbing property and heat stability, and thus can be used for various applications.

A content of the specific compound in the composition is appropriately selected according to the intended use of the composition.

Since the specific compound has favorable affinity, compatibility, and dispersibility with various solvents, resins, and the like, the composition according to the present disclosure may be an aqueous composition or a non-aqueous composition.

Hereinafter, each component contained in the composition according to the present disclosure will be described.

### Aqueous Composition

### (Aqueous Solvent)

In a case where the composition according to the present disclosure is an aqueous composition, the composition can contain an aqueous solvent. The composition may be obtained by dissolving or dispersing the above-described specific compound in an aqueous vehicle.

Examples of the aqueous solvent include water, a hydrophilic organic solvent, and a mixed solvent containing water as a main component, to which a hydrophilic organic solvent is added.

In the present disclosure, the “solvent containing water as a main component” refers to a solvent containing 30% by mass or more of water with respect to the total aqueous solvent.

Examples of the hydrophilic organic solvent include:
alcohols such as methanol, ethanol, propanol, isopropanol, butanol, isobutanol, sec-butanol, tert-butanol, pentanol, hexanol, cyclohexanol, and benzyl alcohol;
polyhydric alcohols such as ethylene glycol, diethylene glycol, triethylene glycol, polyethylene glycol, propylene glycol, dipropylene glycol, polypropylene glycol, butylene glycol, hexanediol, pentanediol, glycerin, hexanetriol, and thiodiglycol;
glycol derivatives such as ethylene glycol monomethyl ether, ethylene glycol monoethyl ether, ethylene glycol monobutyl ether, diethylene glycol monomethyl ether, diethylene glycol monobutyl ether, propylene glycol monomethyl ether, propylene glycol monobutyl ether, dipropylene glycol monomethyl ether, triethylene glycol monomethyl ether, ethylene glycol diacetate, ethylene glycol monomethyl ether acetate, triethylene glycol monoethyl ether, and ethylene glycol monophenyl ether;
amines such as ethanolamine, diethanolamine, triethanolamine, N-methyl diethanolamine, N-ethyl diethanolamine, morpholine, N-ethylmorpholine, ethylene diamine, diethylene triamine, triethylene tetramine, polyethyleneimine, and tetramethyl propylene diamine; and
formamide, N,N-dimethyl formamide, N,N-dimethyl acetamide, dimethylsulfoxide, sulfolane, 2-pyrrolidone, N-methyl-2-pyrrolidone, N-vinyl-2-pyrrolidone, 2-oxazolidone, 1,3-dimethyl-2-imidazolidinone, acetonitrile, and acetone.

The aqueous solvent preferably contains water in an amount of 30% by mass to 100% by mass, more preferably 50% by mass to 100% by mass, with respect to the total amount of all solvents.

### (Aqueous Resin)

The aqueous composition may further contain an aqueous resin. Examples of the aqueous resin include a water-soluble resin which has solubility in water, a water-dispersible resin which has dispersibility in water, a colloidal dispersion resin, and a mixture of these resins.

Specific examples of the aqueous resin include various aqueous resins such as an acrylic resin, a styrene-acrylic resin, a vinyl resin, a polyurethane resin, a polyester resin, a polyamide resin, and a fluororesin. In addition, examples of the water-soluble resin which has solubility in water and is an embodiment of the aqueous resin include gelatin, polyvinyl alcohol, and carboxymethyl cellulose.

The above-described aqueous resin may be used as a binder resin.

Since the specific compound according to the present disclosure has favorable solubility in a solvent, the composition can be used as a composition containing a binder resin to form a near infrared light absorbing layer.

The aqueous composition according to the present disclosure may contain only one kind of the aqueous resin, or may contain two or more kinds thereof.

In a case where the aqueous resin is a binder resin used for forming a layer, a molecular weight of the polymer is not particularly limited, but is preferably approximately 3,000 to 1,000,000 in terms of weight-average molecular weight. In a case where the weight-average molecular weight is within the above-described range, a strength of the coating layer obtained by using the composition according to the present disclosure is sufficient, and thus coating surface properties are improved.

### (Non-Aqueous Composition)

In a case where the composition according to the present disclosure is a non-aqueous composition, the composition may be obtained by dissolving or dispersing the above-described specific compound in a non-aqueous vehicle.

Examples of a resin used as the non-aqueous vehicle include a petroleum resin, casein, shellac, a rosin-modified maleic acid resin, a rosin-modified phenol resin, nitrocellulose, cellulose acetate butyrate, cyclized rubber, chlorinated rubber, rubber oxide, rubber chloride, a phenol resin, an alkyd resin, a polyester resin, an unsaturated polyester resin, an amino resin, an epoxy resin, a vinyl resin, vinyl chloride, a vinyl chloride-vinyl acetate copolymer, an acrylic resin, a methacrylic resin, a polyurethane resin, a silicone resin, a fluororesin, a drying oil, a synthetic drying oil, a styrene/maleic acid resin, a styrene/acrylic resin, a polyamide resin, a polyimide resin, a benzoguanamine resin, a melamine resin, a urea resin, chlorinated polypropylene, a butyral resin, and a vinylidene chloride resin.

A photocurable resin or a thermosetting resin may be used as the non-aqueous vehicle.

In preparation of the non-aqueous composition, examples of a solvent used for dissolving or dispersing the non-aqueous vehicle include aromatic solvents such as toluene, xylene, and methoxybenzene; acetic acid ester-based solvents such as ethyl acetate, butyl acetate, propylene glycol monomethyl ether acetate, and propylene glycol monoethyl ether acetate; propionate-based solvents such as ethoxyethyl propionate; alcohol-based solvents such as methanol and ethanol; ether-based solvents such as butyl cellosolve, propylene glycol monomethyl ether, diethylene glycol ethyl ether, and diethylene glycol dimethyl ether; ketone-based solvents such as methyl ethyl ketone, methyl isobutyl ketone, and cyclohexanone; aliphatic hydrocarbon-based solvents such as hexane; nitrogen compound-based solvents such as N,N-dimethylformamide, γ-butyrolactam, N-methyl-2-pyrrolidone, aniline, and pyridine; lactone-based solvents such as γ-butyrolactone; and carbamic acid esters such as a mixture of methyl carbamate and ethyl carbamate at a ratio of 18:52.

In a case where the composition according to the present disclosure contains the above-described specific compound and an aqueous or non-aqueous medium, the composition can also be prepared by dispersing the specific compound and the medium using a dispersion apparatus.

As the dispersion apparatus which can be used for preparing the dispersion, a known dispersion apparatus can be appropriately selected and used. Examples of the dispersion apparatus include a ball mill, a sand mill, a beads mill, a roll mill, a jet mill, a paint shaker, an attritor, an ultrasonic disperser, and a disperser.

The composition according to the present disclosure has favorable near infrared absorbing property and favorable heat stability, and thus can be used for various applications.

The applications of the composition according to the present disclosure are not particularly limited, and the composition can be used for an ink, an optical filter such as an infrared cut filter, a staining solution for a fiber, photothermal conversion, and the like.

In addition to the above-described applications, the composition according to the present disclosure can also be applied to a diagnostic marker or photodynamic therapy because the composition has absorption in the near infrared region having excellent human body permeability.

The formulation of the composition according to the present disclosure is appropriately adjusted according to the use application. The composition according to the present disclosure can contain various compounds within a range in which the effect of the specific compound according to the present disclosure, which is excellent in near infrared absorbing property, is not impaired.

It is preferable that the coloring agent composition according to the present disclosure further contains a resin. That is, the coloring agent composition according to the present disclosure is preferably a resin composition.

The specific compound according to the present disclosure has excellent absorption ability in a near infrared region having a wavelength of more than 1,150 nm and has favorable heat resistance. Therefore, the composition according to the present disclosure, containing the specific compound, can be made into a resin composition having excellent absorption ability in the near infrared region having a wavelength of more than 1,150 nm and having excellent heat resistance.

Hereinafter, the composition according to the present disclosure, containing a resin, may be referred to as “resin composition according to the present disclosure”.

The resin composition according to the present disclosure may be a composition in a solution state containing a solvent.

In addition, the resin composition according to the present disclosure may be a kneaded material.

In the present disclosure, the kneaded material refers to a material obtained by kneading the specific compound and the resin. That is, the kneaded material in the present disclosure refers to a composition in which the specific compound is mixed and dispersed in the resin, and is different from a liquid composition in which the specific compound and the resin are dissolved or dispersed in a solvent.

It is also preferable that the kneaded material as the resin composition according to the present disclosure is a pellet. Here, the pellet is a material obtained by granulating (pelletizing) the kneaded material into a certain shape such as a spherical shape, an ellipsoidal shape, a cylindrical shape, and a prismatic shape. In addition, it is also preferable that the pellet is a master pellet. The master pellet is also referred to as a master batch. The master pellet is generally a pellet containing a desired compound to be kneaded with a resin at a high concentration, and refers to a pellet obtained by kneading the resin with the compound to adjust the compound in the resin to a desired concentration.

The master pellet in the present disclosure refers to a material in which a high concentration of the specific compound and other components, for example, an additive such as an ultraviolet absorbent are dispersed in a resin, and is used for mixing the resin and the like and the master pellet at a specified magnification in a case of forming a molded body, and preparing the resin and the like, containing the specific compound and the like at a predetermined concentration.

The resin composition according to the present disclosure may contain only one kind of the above-described specific compound, or may contain two or more kinds thereof.

A content of the specific compound in the total solid content of the above-described resin composition is preferably 0.01% by mass to 20% by mass.

The content of the specific compound is more preferably 0.05% by mass or more, and still more preferably 0.1% by mass or more with respect to the total solid content of the resin composition.

The content of the specific compound is more preferably 15% by mass or less, and still more preferably 10% by mass or less with respect to the total solid content of the resin composition.

Among these, in a case where the resin composition according to the present disclosure is used as the kneaded material, from the viewpoint of further improving uniform dispersibility of the specific compound in the resin, the content of the specific compound in the resin composition is preferably 0.01% by mass to 5% by mass or less and more preferably 0.01% by mass to 2% by mass or less in a certain embodiment.

In a case where the above-described resin composition contains two or more kinds of the specific compounds, the above-described content refers to the total amount of the two or more kinds of the specific compounds.

### (Resin)

The resin contained in the resin composition according to the present disclosure will be described.

The resin can be appropriately selected from resins which satisfy various physical properties such as transparency, refractive index, and workability, which are required according to the intended use or purpose.

Examples of the resin include a (meth)acrylic resin, an ene-thiol resin, a polyester resin, a polycarbonate resin, a vinyl polymer (for example, a polydiene resin, a polyalkene resin, a polystyrene resin, a polyvinyl ether resin, a polyvinyl alcohol resin, a polyvinyl ketone resin, a polyfluorovinyl resin, a polyvinyl bromide resin, and the like), a polythioether resin, a polyphenylene resin, a polyurethane resin, a polysulfonate resin, a nitroso polymer resin, a polysiloxane resin, a polysulfide resin, a polythioester resin, a polysulfone resin, a polysulfonamide resin, a polyamide resin, a polyimine resin, a polyurea resin, a polyphosphazene resin, a polysilane resin, a polysilazane resin, a polyfuran resin, a polybenzoxazole resin, a polyoxadiazole resin, a polybenzothiazinophenothiazine resin, a polybenzothiazole resin, a polypyrazinoquinoxaline resin, a polyquinoxaline resin, a polybenzimidazole resin, a polyoxoisoindoline resin, a polydioxoisoindoline resin, a polytriazine resin, a polypyridazine resin, a polypiperazine resin, a polypyridine resin, a polypiperidine resin, a polytriazole resin, a polypyrazole resin, a polypyrrolidine resin, a polycarborane resin, a polyoxabicyclononane resin, a polydibenzofuran resin, a polyphthalide resin, a polyacetal resin, a polyimide resin, a polyamide imide resin, an olefin resin, a cyclic olefin resin, an epoxy resin, and a cellulose acylate resin.

Examples of the (meth)acrylic resin include polymers including a structural unit derived from (meth)acrylic acid and/or an ester thereof. Specific examples thereof include polymers obtained by polymerizing at least one compound selected from the group consisting of (meth)acrylic acid, (meth)acrylic acid ester, (meth)acrylamide, and (meth)acrylonitrile.

Examples of the polyester resin include polymers obtained by reacting a polyol (such as ethylene glycol, propylene glycol, glycerin, and trimethylolpropane) with a polybasic acid (such as aromatic dicarboxylic acid (for example, terephthalic acid, isophthalic acid, naphthalenedicarboxylic acid, and dicarboxylic acid in which a hydrogen atom of these aromatic rings is replaced with a methyl group, an ethyl group, or a phenyl group), aliphatic dicarboxylic acid having 2 to 20 carbon atoms (for example, adipic acid, sebacic acid, and dodecanedicarboxylic acid), and alicyclic dicarboxylic acid (for example, cyclohexanedicarboxylic acid)); and polymers obtained by ring-opening polymerization of a cyclic ester compound such as caprolactone monomers (for example, polycaprolactone). Specific examples of the polyester resin include polyethylene terephthalate and polyethylene naphthalate.

Examples of the epoxy resin include bisphenol A-type epoxy resin, bisphenol F-type epoxy resin, phenol novolac-type epoxy resin, cresol novolac-type epoxy resin, and aliphatic epoxy resin.

As the epoxy resin, a commercially available product on the market may be used, and examples of the commercially available product include the following.

Examples of a commercially available product of the bisphenol A-type epoxy resin include jER825, jER827, jER828, jER834, jER 1001, jER1002, jER1003, jER1055, jER1007, jER 1009, and jER 1010 (all manufactured by Mitsubishi Chemical Corporation); and EPICLON 860, EPICLON 1050, EPICLON 1051, and EPICLON 1055 (all manufactured by DIC Corporation).

Examples of a commercially available product of the bisphenol F-type epoxy resin include jER806, jER807, jER4004, jER4005, jER4007, and jER4010 (all manufactured by Mitsubishi Chemical Corporation); EPICLON (registered trademark) 830 and EPICLON 835 (both manufactured by DIC Corporation); and LCE-21 and RE-602S (both manufactured by Nippon Kayaku Co., Ltd.).

Examples of a commercially available product of the phenol novolac-type epoxy resin include jER152, jER154, jER157S70, and jER157S65 (all manufactured by Mitsubishi Chemical Corporation); and EPICLON N-740, EPICLON N-770, and EPICLON N-775 (all manufactured by DIC Corporation).

Examples of a commercially available product of the cresol novolac-type epoxy resin include EPICLON N-660, EPICLON N-665, EPICLON N-670, EPICLON N-673, EPICLON N-680, EPICLON N-690, and EPICLON N-695 (all manufactured by DIC Corporation); and EOCN-1020 (manufactured by Nippon Kayaku Co., Ltd.).

Examples of a commercially available product of the aliphatic epoxy resin include ADEKA RESIN EP series (for example, EP-4080S, EP-4085S, and EP-4088S; manufactured by ADEKA Corporation); CELLOXIDE (registered trademark) 2021P, CELLOXIDE 2081, CELLOXIDE 2083, CELLOXIDE 2085, EHPE 3150, EPOLEAD PB 3600, and EPOLEAD PB 4700 (all manufactured by Daicel Corporation); DENACOL EX-212L, EX-214L, EX-216L, EX-321L, and EX-850L (all manufactured by Nagase ChemteX Corporation); ADEKA RESIN EP series (for example, EP-4000S, EP-4003S, EP-4010S, and EP-4011S; manufactured by ADEKA Corporation); NC-2000, NC-3000, NC-7300, XD-1000, EPPN-501, and EPPN-502 (all manufactured by ADEKA Corporation); and jER1031S (manufactured by Mitsubishi Chemical Corporation).

Other examples of the commercially available product of the epoxy resin include MARPROOF G-0150M, G-0105SA, G-0130SP, G-0250SP, G-1005S, G-1005SA, G-1010S, G-2050M, G-01100, and G-01758 (all manufactured by NOF Corporation, epoxy group-containing polymer).

As the cellulose acylate resin, cellulose acylates described in paragraphs to of JP2012-215689A are preferably used.

As the polyester resin, a commercially available product such as VYLON (registered trademark) series (for example, VYLON 500) manufactured by Toyobo Co., Ltd. can also be used.

As a commercially available product of the (meth)acrylic resin, SK Dyne series (for example, SK Dyne-SF2147) manufactured by Soken Chemical & Engineering Co., Ltd. can also be used.

As the polystyrene resin, a resin including 50% by mass or more of a repeating unit derived from a styrene-based monomer is preferable; a resin including 70% by mass or more of a repeating unit derived from a styrene-based monomer is more preferable; and a resin including 85% by mass or more of a repeating unit derived from a styrene-based monomer is still more preferable.

Specific examples of the styrene-based monomer include styrene and styrene derivatives. Here, the styrene derivative is a compound in which another group is bonded to styrene, and examples thereof include alkylstyrene such as o-methylstyrene, m-methylstyrene, p-methylstyrene, 2,4-dimethylstyrene, o-ethylstyrene, and p-ethylstyrene, and substituted styrene in which a hydroxyl group, an alkoxy group, a carboxyl group, or halogen is introduced to a benzene nucleus of styrene such as hydroxystyrene, tert-butoxystyrene, vinyl benzoic acid, o-chlorostyrene, and p-chlorostyrene.

In addition, the polystyrene resin may include a repeating unit derived from a monomer other than the styrene-based monomer. Examples of other monomers include alkyl (meth)acrylate such as methyl (meth)acrylate, cyclohexyl (meth)acrylate, methylphenyl (meth)acrylate, or isopropyl (meth)acrylate; an unsaturated carboxylic acid monomer such as methacrylic acid, acrylic acid, itaconic acid, maleic acid, fumaric acid, and cinnamic acid; an unsaturated dicarboxylic acid anhydride monomer which is an anhydride of maleic acid anhydride, itaconic acid, ethylmaleic acid, methylitaconic acid, or chloromaleic acid; an unsaturated nitrile monomer such as acrylonitrile and methacrylonitrile; and a conjugated diene such as 1,3-butadiene, 2-methyl-1,3-butadiene (isoprene), 2,3-dimethyl-1,3-butadiene, 1,3-pentadiene, and 1,3-hexadiene.

Examples of a commercially available product of the polystyrene resin include AS-70 (acrylonitrile-styrene copolymer resin) manufactured by NIPPON STEEL Chemical & Material Co., Ltd.; SMA2000P (styrene-maleic acid copolymer) manufactured by KAWAHARA PETROCHEMICAL CO., LTD.; CLEAREN 530L and CLEAREN 730L manufactured by Denka Company Limited; TUFPRENE 126S and ASAPRENE T411 manufactured by Asahi Kasei Corporation; KRATON D1102A and KRATON D1116A manufactured by Kraton Corporation; STYROLUX S and STYROLUX T manufactured by INEOS Styrolution Group GmbH; ASAFLEX 840 and ASAFLEX 860 manufactured by Asahi Kasei Corporation; 679, HF77, SGP-10, 475D, H0103, and HT478 manufactured by PS Japan Corporation; and DICSTYRENE XC-515, DICSTYRENE XC-535, and DICSTYRENE GH-8300-5 manufactured by DIC Corporation. In addition, examples of a commercially available product of the hydrogenated polystyrene resin include TUFTEC H series manufactured by Asahi Kasei Corporation, KRATON G series manufactured by Shell Japan Limited, DYNARON (hydrogenated styrene-butadiene random copolymer) manufactured by JSR Corporation, and SEPTON manufactured by Kuraray Co., Ltd. In addition, examples of a commercially available product of the modified polystyrene resin include TUFTEC M series manufactured by Asahi Kasei Corporation, EPOFRIEND manufactured by Daicel Corporation, polar group-modified DYNARON manufactured by JSR Corporation, and RESEDA manufactured by TOAGOSEI CO., LTD.

Examples of the cyclic olefin resin include (R1) polymers including a structural unit derived from a norbornene compound, (R2) polymers including a structural unit derived from a monocyclic olefin compound which is not a norbornene compound, (R3) polymers including a structural unit derived from a cyclic conjugated diene compound, (R4) polymers including a structural unit derived from a vinyl alicyclic hydrocarbon compound, and hydrides of polymers including a structural unit derived from each of the compounds (R1) to (R4).

In the present disclosure, the polymer including a structural unit derived from a norbornene compound and the polymer including a structural unit derived from a monocyclic olefin compound are used in a sense of including ring-opening polymers of the respective compounds.

The cyclic olefin resin is not particularly limited, but is preferably a polymer having a structural unit derived from a norbornene compound, which is represented by Formula (A-II) or Formula (A-III). The polymer having the structural unit represented by Formula (A-II) is an addition polymer of a norbornene compound, and the polymer having the structural unit represented by Formula (A-III) is a ring-opening polymer of a norbornene compound.

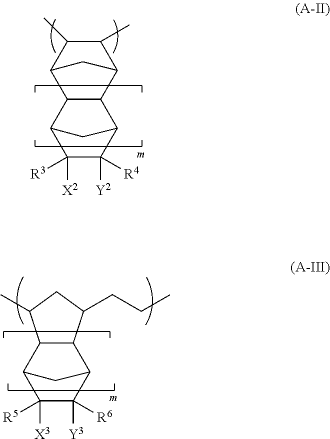

In Formulae (A-II) and (A-III), m represents an integer of 0 to 4 and is preferably 0 or 1.

R³ to R⁶ in Formulae (A-II) and (A-III) each independently represent a hydrogen atom or a hydrocarbon group having 1 to 10 carbon atoms.

Examples of the hydrocarbon group represented by R³ to R⁶ include an alkyl group, an alkenyl group, an alkynyl group, and an aryl group, and an alkyl group or an aryl group is preferable.

X² and X³, and Y² and Y³ each independently represent a hydrogen atom, a hydrocarbon group having 1 to 10 carbon atoms, a halogen atom, a hydrocarbon group having 1 to 10 carbon atoms, which is substituted with a halogen atom, —(CH₂),COOR₁₁, —(CH₂)ₙOCOR₁₂, —(CH₂)ₙNCO, —(CH₂)ₙNO₂, —(CH₂)ₙCN, —(CH₂)ₙCONR¹³R¹⁴, —(CH₂)ₙNR¹³R¹⁴, —(CH₂)ₙOZ¹, —(CH₂)ₙW¹, or (—CO)₂O or (—CO)₂NR¹⁵ which is formed by bonding X² and Y², or X³ and Y³.

Here, R¹¹ to R¹⁵ in the above-described groups which can be adopted as X², X³, Y², and Y³ each independently represent a hydrogen atom or a hydrocarbon group having 1 to 20 carbon atoms, Z¹ represents a hydrocarbon group or a hydrocarbon group substituted with halogen, and W¹ represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p represents an integer of 0 to 3). n represents an integer of 0 to 10, and is preferably 0 to 8 and more preferably 0 to 6.

In Formulae (A-II) and (A-III), R³ to R⁶ are each independently preferably a hydrogen atom or —CH₃, and from the viewpoint of moisture permeability, still more preferably a hydrogen atom.

Each of X² and X³ is preferably a hydrogen atom, —CH₃, or —C₂H₅, and from the viewpoint of moisture permeability, still more preferably a hydrogen atom.

Y² and Y³ are each independently preferably a hydrogen atom, a halogen atom (particularly a chlorine atom), or —(CH₂)ₙCOOR¹¹ (particularly —COOCH₃), and from the viewpoint of moisture permeability, still more preferably a hydrogen atom.

Other groups are appropriately selected.

The polymer having the structural unit represented by Formula (A-II) or Formula (A-III) may further include one or more of a structural unit represented by Formula (A-I).

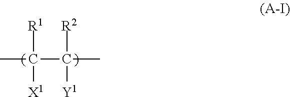

In Formula (A-I), R¹ and R² each independently represent a hydrogen atom or a hydrocarbon group having 1 to 10 carbon atoms, and X¹ and Y¹ each independently represent a hydrogen atom, a hydrocarbon group having 1 to 10 carbon atoms, a halogen atom, a hydrocarbon group having 1 to 10 carbon atoms, which is substituted with a halogen atom, —(CH₂)ₙCOOR₁₁, —(CH₂)ₙOCOR₁₂, —(CH₂)ₙNCO, —(CH₂)ₙNO₂, —(CH₂)ₙCN, —(CH₂)ₙCONR¹³R¹⁴, —(CH₂)ₙNR¹³R¹⁴, —(CH₂)ₙOZ¹, —(CH₂)ₙW¹, or (13 CO)₂O or (—CO)₂NR¹⁵ formed by X² and Y² or X³ and Y³ being bonded to each other. R¹¹ to R¹⁵ in the above-described groups which can be adopted as X¹ and Y¹ each independently represent a hydrogen atom or a hydrocarbon group having 1 to 20 carbon atoms, Z¹ represents a hydrocarbon group or a hydrocarbon group substituted with halogen, and W¹ represents Si(R¹⁶)ₚD₍₃₋ₚ₎ (R¹⁶ represents a hydrocarbon group having 1 to 10 carbon atoms, and D represents a halogen atom, —OCOR¹⁷, or —OR¹⁷ (R¹⁷ represents a hydrocarbon group having 1 to 10 carbon atoms), and p represents an integer of 0 to 3). n represents an integer of 0 to 10.

A content of the structural unit represented by Formula (A-II) or Formula (A-III) in the cyclic polyolefin resin is preferably 90% by mass or less, more preferably 30% by mass to 85% by mass, still more preferably 50% by mass to 79% by mass, and even more preferably 60% by mass to 75% by mass.

The cyclic olefin resin is described in JP1998-007732A (JP-H10-007732A), JP2002-504184A, WO2004/070463A, and the like, the contents of which can be referred to as appropriate.

The cyclic olefin resin is obtained by performing an addition polymerization of a norbornene compound (for example, a polycyclic unsaturated compound of norbornene) with each other.

As the cyclic olefin resin, a commercially available product may be used, and examples of the commercially available product of the cyclic olefin resin include ARTON series (for example, ARTON G, ARTON F, and ARTON RX4500) manufactured by JSR Corporation, and Zeonor ZF14, Zeonor ZF16, Zeonex 250, and Zeonex 280 (manufactured by Zeon Corporation).

In addition, examples of the cyclic olefin resin include copolymers obtained by an addition copolymerization of, as necessary, a norbornene compound, and olefin such as ethylene, propylene, and butene, conjugated diene such as butadiene and isoprene, unconjugated diene such as ethylidene norbornene, or an ethylenically unsaturated compound such as acrylonitrile, acrylic acid, methacrylic acid, maleic acid anhydride, acrylic acid ester, methacrylic acid ester, maleimide, vinyl acetate, and vinyl chloride; and among these, a copolymer with ethylene is preferable.

Examples of the addition (co)polymers of a norbornene compound include APL8008T (Tg: 70° C.), APL6011T (Tg: 105° C.), APL6013T (Tg: 125° C.), and APL6015T (Tg: 145° C.) which are sold by Mitsui Chemicals, Inc. under a trade name of APL and have different glass transition temperatures (Tg). In addition, pellets such as TOPAS8007, TOPAS6013, and TOPAS6015 are commercially available from Polyplastics Co., Ltd. Furthermore, Appear3000 is commercially available from Film Ferrania S. R. L.

In addition, the hydride of the cyclic olefin resin can be synthesized by an addition polymerization or a ring-opening metathesis polymerization of a norbornene compound or the like and then an addition of hydrogen. The synthesis method is described, for example, in JP1989-240517A (JP-H01-240517A), JP1995-196736A (JP-H07-196736A), JP1985-026024A (JP-S60-026024A), JP1987-019801A (JP-S62-019801A), JP2003-159767A, and JP2004-309979A.

A weight-average molecular weight of the cyclic olefin resin contained in the resin composition according to the present disclosure is preferably 5,000 to 500,000, more preferably 8,000 to 200,000, and still more preferably 10,000 to 100,000.

Examples of the polycarbonate resin include a reaction product of a polyhydric phenol compound and phosgene or a carbonic ester compound.

Examples of the polyhydric phenol compound include hydroquinone, resorcinol, 4,4′-dihydroxydiphenyl, bis(4-hydroxyphenyl)methane, 1,1-bis(4-hydroxyphenyl)ethane, 1,1-bis(4-hydroxyphenyl)-1-phenylethane, bisphenol A, bisphenol C, bisphenol E, bisphenol F, bisphenol M, bisphenol P, bisphenol S, bisphenol Z, 2,2-bis(3-methyl-4-hydroxyphenyl)propane, 1,1-bis(4-hydroxyphenyl)cyclohexane, 2,2-bis(3-phenyl-4-hydroxyphenyl)propane, 2,2-bis(3-isopropyl-4-hydroxyphenyl)propane, 2,2-bis(4-hydroxyphenyl)butane, 2,2-bis(3,5-dimethyl-4-hydroxyphenyl)propane, 2,2-bis(3,5-dibromo-4-hydroxyphenyl)propane, 4,4′-dihydroxydiphenyl sulfone, 4,4′-dihydroxydiphenyl sulfoxide, 4,4′-dihydroxydiphenyl sulfide, 3,3′-dimethyl-4,4′-dihydroxydiphenyl sulfide, and 4,4′-dihydroxydiphenyl oxide; and among these, hydroquinone, resorcinol, 4,4′-dihydroxydiphenyl, or bisphenol A is preferable.

Examples of the carbonic ester compound include phosgene, diphenyl carbonate, bis(chlorophenyl) carbonate, dinaphthyl carbonate, bis(diphenyl) carbonate, dimethyl carbonate, diethyl carbonate, and dibutyl carbonate; and bis(diphenyl) carbonate, dimethyl carbonate, or diethyl carbonate is preferable.

Examples of a commercially available product of the polycarbonate resin include PANLITE L-1250WP and PANLITE SP-1516 manufactured by Teijin Limited; Iupizeta EP-5000 and Iupizeta EP-4000 manufactured by Mitsubishi Gas Chemical Company Inc.; and Calibre 301-30 manufactured by Sumika Polycarbonate Ltd.

Examples of the thiourethane resin include a reaction product of an isocyanate compound and a polythiol compound and a reaction product of a thiourethane resin precursor. Examples of a commercially available product of the thiourethane resin precursor include MR-7, MR-8, MR-10, and MR-174 manufactured by Mitsui Chemicals, Inc.

Examples of the polyamide resin include an aliphatic polyamide resin and an aromatic polyamide resin. Examples of the aliphatic polyamide resin include Nylon 6, Nylon 11, Nylon 12, Nylon 46, Nylon 66, Nylon 666, Nylon 610, and Nylon 612. Examples of the aromatic polyamide resin include a resin which is polymerized by dehydration condensation of a diamine and a dicarboxylic acid, in which at least one of the diamine or the dicarboxylic acid includes an aromatic ring. Specific examples of the aromatic polyamide resin include a condensation polymer of m-xylylenediamine and adipic acid or an adipic acid halide.

The resin is preferably at least one selected from a (meth)acrylic resin, a polystyrene resin, a polyester resin, a polyurethane resin, a thiourethane resin, a polyimide resin, a polyamide resin, an epoxy resin, a polycarbonate resin, a phthalate resin, a cellulose acylate resin, or a cyclic olefin resin; and from the viewpoint that compatibility with the specific compound is favorable and a cured substance in which surface unevenness is suppressed is easily obtained, it is more preferable to be at least one resin selected from a (meth)acrylic resin, a polystyrene resin, a polyester resin, a polyurethane resin, a cyclic olefin resin, or a polycarbonate resin.

A weight-average molecular weight (Mw) of the resin contained in the resin composition according to the present disclosure is preferably 2,000 to 2,000,000. Mw of the resin is preferably 5,000 or more, more preferably 10,000 or more, and still more preferably 50,000 or more. Mw of the resin is preferably 1,000,000 or less, more preferably 500,000 or less, and still more preferably 200,000 or less.

In addition, in a case of using an epoxy resin as the resin, a weight-average molecular weight (Mw) of the epoxy resin is preferably 100 or more, and more preferably 200 to 2,000,000. Mw of the epoxy resin is preferably 1,000,000 or less and more preferably 500,000 or less. Mw of the epoxy resin is preferably 2000 or more.

In the present disclosure, the weight-average molecular weight (Mw) of the resin is a value measured by gel permeation chromatography (GPC).

In the measurement by GPC, HLC (registered trademark)-8020GPC (manufactured by Tosoh Corporation) is used as a measuring device, three columns of TSKgel (registered trademark) Super Multipore HZ-H (4.6 mmID×15 cm, manufactured by Tosoh Corporation) are used as a column, and tetrahydrofuran (THF) is used as an eluent.

In addition, as the measurement conditions, a sample concentration of 0.45% by mass, a flow rate of 0.35 ml/min, a sample injection amount of 10 μl (microliters), and a measurement temperature of 40° C. are set, and a RI detector is used.

The calibration curve is created from eight samples of “Standard sample TSK standard, polystyrene” manufactured by Tosoh Corporation: “F-40”, “F-20”, “F-4”, “F-1”, “A-5000”, “A-2500”, “A-1000”, and “n-propylbenzene”.

The resin composition according to the present disclosure may contain only one kind of the resin, or may contain two or more kinds thereof.

A content of the resin in the resin composition according to the present disclosure may be appropriately determined according to the purpose of use, application, and the like of the resin composition according to the present disclosure.

### (Other Components)

The composition according to the present disclosure may further contain other components in addition to the above-described specific compound and various solvents and resins which may be contained as desired, as long as the effects of the near infrared absorbing property, the heat stability, and the like of the composition are not impaired.

Examples of the other components include an infrared absorber other than the above-described specific compound (also referred to as “other infrared absorbers”), a surfactant for improving the coating surface properties during the formation of a film, a polymerizable compound, a pigment derivative, a polymerization inhibitor, a solvent, a sensitizer, a co-sensitizer, an adhesion promoter, an antioxidant, an ultraviolet absorber, and an aggregation inhibitor.

### (Other Infrared Absorbers)

The other infrared absorbers are preferably a near infrared absorber other than the above-described specific compound.

Examples of other near infrared absorbers include a pyrrolopyrrole compound, a squarylium compound, a cyanine compound, a phthalocyanine compound, a naphthalocyanine compound, a quaterrylene compound, a merocyanine compound, a croconium compound, an oxonol compound, an iminium compound, a dithiol compound, a triarylmethane compound, a pyrromethene compound, an azomethine compound, an anthraquinone compound, a dibenzofuranone compound, a metal oxide, and a metal boride.

In a case where the present disclosure includes the other infrared absorbers, the infrared absorber may be contained alone or in combination of two or more kinds thereof. A content of the other infrared absorbers is preferably less than 100% by mass, more preferably 50% by mass or less, and still more preferably 0% by mass with respect to the total amount of the specific compound according to the present disclosure and the other infrared absorbers.

### (Polymerizable Compound)

The composition according to the present disclosure may contain a polymerizable compound. In a case where the composition according to the present disclosure further contains a polymerizable compound, the composition can be used as a composition having pattern formability in which only an energy-applied portion is cured by local energy application such as pattern exposure.

### Image Forming Material

The image forming material according to the present disclosure contains the compound represented by General Formula (1) described above (the specific compound (1) according to the present disclosure).

The image forming material according to the present disclosure can be suitably used as an image recording material which absorbs light in the near infrared region.

Specific examples of the image forming material include an ink jet recording material, a heat-sensitive recording material, a pressure-sensitive recording material, a recording material by an electrophotographic method, a transfer type silver halide photosensitive material, a printing ink, a recording pen, and Japanese stamp. Among these, the image forming material according to the present disclosure can be particularly suitably used as an ink jet recording material or a recording material by an electrophotographic method.

The image forming material according to the present disclosure preferably contains a liquid medium.

The liquid medium is not particularly limited, and for example, the same solvent as in the composition according to the present disclosure can be used. The liquid medium in a case where the image forming material according to the present disclosure is an aqueous composition will be described later.

The image forming material according to the present disclosure may contain the compound according to the present disclosure in a state of being dissolved in the liquid medium or may contain the compound according to the present disclosure in a state of being dispersed in the liquid medium as solid particles.

The image forming material containing the compound according to the present disclosure as solid particles can be prepared by dispersing the compound according to the present disclosure in the liquid medium using a dispersion apparatus.

The dispersion apparatus is not particularly limited, and a known dispersion apparatus in the related art can be used.

Specific examples of the dispersion apparatus include a ball mill, a sand mill, a beads mill, a roll mill, a jet mill, a paint shaker, an attritor, an ultrasonic disperser, and a disperser.

A volume average particle size of the particles is not particularly limited, and is, for example, preferably 10 nm to 250 nm, more preferably 20 nm to 250 nm, and still more preferably 30 nm to 230 nm.

In a case where the volume average particle size of the particles is within the above-described range, storage stability of the image forming material is further improved and a sufficient optical density can be obtained.

The volume average particle size of the particles is measured using a particle size distribution measuring device adopting a dynamic light scattering method. For example, a particle size distribution measuring device (trade name: UPA-EX150, manufactured by MicrotracBEL Corp.) can be used as the measuring device. However, the measuring device is not limited thereto.

The image forming material according to the present disclosure may contain only one type of the compound according to the present disclosure, or may contain two or more types of the compounds according to the present disclosure.

A content of the compound according to the present disclosure in the image forming material is not particularly limited, and can be appropriately set depending on the intended purpose. In general, from the viewpoint of sufficiently exhibiting the infrared absorbing ability, the content of the compound according to the present disclosure in the image forming material is preferably 0.001% by mass to 30% by mass, more preferably 0.01% by mass to 10% by mass, and still more preferably 0.05% by mass to 5% by mass with respect to the total mass of the image forming material.

In a case where the image forming material according to the present disclosure is an aqueous composition, examples of the liquid medium include water and a mixed liquid of water and an organic solvent.

A content of the water in the liquid medium is preferably 30% by mass to 100% by mass and more preferably 50% by mass to 100% by mass with respect to the total mass of the liquid medium.

The water is not particularly limited, and is, for example, preferably distilled water, ion exchange water, ion exchange distilled water, or pure water from the viewpoint of containing few impurities.

In a case where the liquid medium contains an organic solvent in addition to water, examples of the organic solvent include alcohol compounds such as methanol, ethanol, propanol, isopropanol, butanol, isobutanol, sec-butanol, tert-butanol, pentanol, hexanol, cyclohexanol, and benzyl alcohol; polyhydric alcohol compounds such as ethylene glycol, diethylene glycol, triethylene glycol, polyethylene glycol, propylene glycol, dipropylene glycol, polypropylene glycol, butylene glycol, hexanediol, pentanediol, glycerin, hexanetriol, and thiodiglycol; glycol derivatives such as ethylene glycol monomethyl ether, ethylene glycol monoethyl ether, ethylene glycol monobutyl ether, diethylene glycol monomethyl ether, diethylene glycol monobutyl ether, propylene glycol monomethyl ether, propylene glycol monoethyl ether, propylene glycol monobutyl ether, dipropylene glycol monomethyl ether, triethylene glycol monomethyl ether, ethylene glycol diacetate, ethylene glycol monomethyl ether acetate, triethylene glycol monoethyl ether, ethylene glycol monophenyl ether, 3-methyl-3-methoxybutanol, and 3-methoxybutanol; amine compounds such as ethanolamine, diethanolamine, triethanolamine, N-methyl diethanolamine, N-ethyl diethanolamine, morpholine, N-ethylmorpholine, ethylene diamine, diethylene triamine, triethylene tetramine, polyethyleneimine, and tetramethyl propylene diamine; and organic solvents having solubility with water, such as formamide, N,N-dimethyl formamide, N,N-dimethyl acetamide, dimethylsulfoxide, sulfolane, 2-pyrrolidone, N-methyl-2-pyrrolidone, N-vinyl-2-pyrrolidone, 2-oxazolidone, 1,3-dimethyl-2-imidazolidinone, acetonitrile, acetone, methyl ethyl ketone, tetrahydrofuran, and butyl cellosolve.

In a case where the image forming material according to the present disclosure is an aqueous composition, the image forming material according to the present disclosure may further contain an aqueous resin.

Examples of the aqueous resin include a resin which has solubility in water, a water-dispersible resin which has dispersibility in water, a colloidal dispersion resin, and a mixture of these resins.

Here, the “resin which has solubility in water” refers to a resin which is dissolved in water at 25° C. in an amount of 1% by mass or more. Specific examples of the resin which has solubility in water include gelatin, a vinyl resin (for example, polyvinyl alcohol), and a water-soluble cellulose derivative (for example, carboxymethyl cellulose).

Examples of the water-dispersible resin include a hydrophobic synthetic resin.

Specific examples of the water-dispersible resin include an acrylic resin, a styrene-acrylic resin, a vinyl resin, a polyurethane, a polyester, a polyamide, and a fluororesin.

Examples of the acrylic resin include a homopolymer or a copolymer obtained by polymerization of at least one monomer selected from the group consisting of acrylic acid, an acrylic acid ester compound (for example, alkyl acrylate), acrylamide, acrylonitrile, methacrylic acid, a methacrylic acid ester compound (for example, alkyl methacrylate), methacrylamide, and methacrylonitrile.

Among these, the acrylic resin is preferably a homopolymer or a copolymer obtained by polymerization of at least one monomer selected from the group consisting of an acrylic acid ester compound and a methacrylic acid ester compound, and more preferably a homopolymer or a copolymer obtained by polymerization of at least one monomer selected from the group consisting of an acrylic acid ester compound having an alkyl group having 1 to 6 carbon atoms and a methacrylic acid ester compound having an alkyl group having 1 to 6 carbon atoms.

In a case where the image forming material according to the present disclosure further contains an aqueous resin, the image forming material according to the present disclosure may contain the aqueous resin in the form of an aqueous dispersion of resin particles.

A commercially available product can be used as the aqueous dispersion of resin particles.

Examples of the commercially available product of the aqueous dispersion of resin particles include SUPERFLEX 830, 460, 870, 420, and 420NS [manufactured by DKS Co. Ltd.; polyurethane], BONDIC 1370NS and 1320NS [manufactured by DIC Corporation; polyurethane], HYDRAN HW140SF, WLS201, WLS202, and WLS213 [manufactured by DIC Corporation; polyurethane], OLESTER UD350, UD500, and UD600 [manufactured by Mitsui Chemicals, Inc.; polyurethane], NEOREZ R972, R966, and R9660 [manufactured by Kusumoto Chemicals, Ltd.; polyurethane], FINETEX ES650 and ES2200 [manufactured by DIC Corporation; polyester], VYLONAL (registered trademark) MD1100, MD1400, and MD1480 [manufactured by Toyobo Co., Ltd.; polyester], JURYMER (registered trademark) ET325, ET410, AT-613, and SEK301 [manufactured by Nihon Junyaku Co., Ltd.; acrylic resin], BONCOAT AN117 and AN226 [manufactured by DIC Corporation; acrylic resin], LACSTAR DS616 and DS807 [manufactured by DIC Corporation; styrene-butadiene rubber], NIPOL LX110, LX206, LX426, and LX433 [manufactured by Zeon Corporation; styrene-butadiene rubber], and NIPOL LX513, LX1551, LX550, and LX1571 [manufactured by Zeon Corporation; acrylonitrile-butadiene rubber].

The image forming material according to the present disclosure preferably further contains a surfactant.

In a case where the image forming material according to the present disclosure further contains a surfactant, for example, the dispersibility of the particles can be improved. In addition, in a case where the image forming material according to the present disclosure further contains a surfactant, for example, quality of an image to be formed can be improved.

The surfactant in the image forming material according to the present disclosure has the same definition as the surfactant in the composition according to the present disclosure, and the same applies to preferred aspects thereof, so that the description thereof will not be repeated here.

In a case where the image forming material according to the present disclosure is an ink, the image forming material according to the present disclosure contains the compound according to the present disclosure and the liquid medium.

Specific examples of the ink include an ink for planographic printing, an ink for ink jetting, an ultraviolet curable ink, an ink for writing instruments (for example, a ballpoint pen), a toner, an ink for vermilion, an ink for penetrating stamps, a textile printing ink, an ink for letterpress printing, an ink for intaglio printing (for example, gravure printing), an ink for stencil printing (for example, screen printing), and a flexographic ink.

In a case where the image forming material according to the present disclosure is an ink, it is preferable that the compound according to the present disclosure is in a state of being dispersed as solid particles in the liquid medium, and the liquid medium is water or a mixed liquid of water and an organic solvent.

In a case where the image forming material according to the present disclosure is an ink, the image forming material according to the present disclosure may contain various additives as necessary as long as the effects of the present disclosure are not impaired.

Examples of the additive include additives such as a resin, an anti-drying agent (so-called wetting agent), an antifading agent, an emulsification stabilizer, a penetration enhancer, a preservative, a fungicide, a pH adjuster, a surface tension adjuster, an antifoaming agent, a viscosity adjuster, a dispersant, a dispersion stabilizer, a rust inhibitor, and a chelating agent.

The antifading agent is used for the purpose of improving the storage stability of the image to be formed by an ink which is the image forming material according to the present disclosure.

Examples of the resin include the same resins as those in the composition according to the present disclosure.

In a case where the image forming material according to the present disclosure is an aqueous composition, the additive can be directly contained in the image forming material according to the present disclosure.

A recording medium in a case where the image is formed using the image forming material according to the present disclosure is not particularly limited; and examples thereof include paper such as ordinary uncoated paper and coated paper, a resin film obtained by forming various non-absorbent resin materials used in so-called soft packaging into a film, and a metal foil.

Specific examples of the paper include pure white roll paper, kraft paper, paperboard, high-quality paper, OCR paper, art paper, coated paper, mirror-coated paper, condenser paper, and paraffin paper.

Specific examples of the resin film include a polyester film, a polypropylene (PP) film, a cellophane, an acetate film, a polycarbonate (PC) film, an acrylic resin film, a polyethylene terephthalate (PET) film, a biaxially oriented polystyrene (OPS) film, a biaxially oriented polypropylene (OPP) film, a biaxially oriented nylon (ONy) film, a polyvinyl chloride (PVC) film, a polyethylene (PE) film, and a triacetate (TAC) film.

In addition, examples of the recording medium include laminated paper obtained by coating paper with a resin and a composite substrate obtained by forming a metal layer of copper, aluminum, or the like on paper or a resin film.

### Film

In a case where the composition according to the present disclosure is the resin composition, the resin contained in the composition is a component which contributes to film-forming properties, and a film is formed by the composition according to the present disclosure by containing the resin.

The film in the present disclosure is the above-described coloring agent composition according to the present disclosure, further including the resin composition containing a resin. The film according to the present disclosure may be a cured substance of the coloring agent composition according to the present disclosure, or a film-like molded product of the coloring agent composition according to the present disclosure. More specifically, the film according to the present disclosure contains the coloring agent composition according to the present disclosure, containing a resin; and examples of one aspect thereof include a film obtained by forming the coloring agent composition according to the present disclosure containing a resin obtained by kneading the specific compound (1), and examples of another aspect thereof include a film which is a cured substance of a coloring agent composition containing the specific compound (1), a resin, and a solvent.

In a case where the resin composition according to the present disclosure contains a solvent, the solvent may be dried to form a film as a cured substance.

In the “drying” in the present disclosure, it is sufficient to remove at least a part of the solvent and it is not necessary to completely remove the solvent, and the amount of the solvent removed can be set as desired.

The film according to the present disclosure can be preferably used as an optical filter such as an infrared cut filter. In addition, the film according to the present disclosure can also be used as a heat ray shielding filter or an infrared transmitting filter.

The film which is a cured substance of the resin composition according to the present disclosure may be a film which is laminated on a support, or may be a self-supporting film which is obtained by curing the resin composition on a support and then peeling off the cured substance from the support.

The film according to the present disclosure may be a film having a pattern or a film (flat film) not having a pattern.

A thickness of the film in the present disclosure is appropriately selected depending on the application of the film, for example, an optical filter, a heat shield film, or a photothermal conversion film.

For example, in a case where the film is used as an infrared absorbing filter, the thickness of the film can be, for example, 0.1 μm to 1,000 μm, preferably 0.5 μm to 500 μm.

The thickness of the film according to the present disclosure can be appropriately adjusted according to the purpose. The thickness of the film is preferably 100 μm or less, more preferably 50 μm or less, and still more preferably 20 μm or less. The lower limit of the thickness of the film is preferably 0.1 μm or more, more preferably 0.2 μm or more, and still more preferably 0.3 μm or more.

### Optical Filter

The optical filter according to the present disclosure includes the above-described film according to the present disclosure.

The optical filter according to the present disclosure can be preferably used as an infrared cut filter or an infrared transmitting filter, and more preferably used as an infrared cut filter.

In addition, a preferred aspect of the optical filter according to the present disclosure is also an aspect of including the film according to the present disclosure and a pixel selected from the group consisting of red, green, blue, magenta, yellow, cyan, black, and colorless.

The infrared cut filter which is one aspect of the optical filter according to the present disclosure includes the film according to the present disclosure.

The infrared cut filter according to the present disclosure may be a filter which cuts only infrared rays having a wavelength of a part of the infrared range, or a filter which cuts the entire infrared range. Examples of the filter which cuts only infrared rays having a wavelength of a part of the infrared range include a near infrared cut filter.

The near infrared cut filter is preferably a filter which cuts infrared rays having a wavelength of 1,000 nm to 2,500 nm, and more preferably a filter which cuts infrared rays having a wavelength in a range of 1,100 nm to 2,000 nm.

The infrared cut filter according to the present disclosure may further include a layer containing copper, a dielectric multi-layer film, an ultraviolet absorbing layer, or the like, in addition to the above-described film. In a case where the infrared cut filter according to the present disclosure further includes at least a layer containing copper or a dielectric multi-layer film, it is easy to obtain an infrared cut filter having a wide viewing angle and excellent infrared shielding property.

In addition, in a case where the infrared cut filter according to the present disclosure further includes an ultraviolet absorbing layer, an infrared cut filter having excellent infrared shielding property can be obtained. The details of the ultraviolet absorbing layer can be found in the description of an absorbing layer described in paragraphs 0040 to 0070 and 0119 to 0145 of WO2015/099060A, the content of which is incorporated herein by reference. The details of the dielectric multi-layer film can be found in paragraphs 0255 to 0259 of JP2014-41318A, the content of which is incorporated herein by reference. As the layer containing copper, a glass base material (copper-containing glass base material) formed of glass containing copper, or a layer (copper complex-containing layer) containing a copper complex may also be used. Examples of the copper-containing glass base material include a phosphate glass containing copper and a fluorophosphate glass containing copper. Examples of a commercially available product of the copper-containing glass include NF-50 (manufactured by AGC Techno Glass Co., Ltd.), BG-60 and BG-61 (both of which are manufactured by Schott AG), and CD5000 (manufactured by Hoya Corporation).

The infrared cut filter according to the present disclosure can be used in various devices including a solid-state imaging element such as a charge coupled device (CCD) or a complementary metal-oxide semiconductor (CMOS), an infrared sensor, or an image display device.

In particular, the infrared cut filter according to the present disclosure is useful for a digital still camera, a smartphone camera, a mobile phone camera, a digital video camera, a camera for a wearable device, a PC camera, a surveillance camera, an automobile camera, a television, car navigation, a mobile information terminal, a video game machine, a portable game machine, a fingerprint recognition system, a digital music player, and the like. Furthermore, the infrared cut filter according to the present disclosure is also useful as a heat ray-cut filter mounted on a glass plate or the like of an automobile, a building, or the like.

It is also preferable that the infrared cut filter according to the present disclosure has a pixel (pattern) of the film obtained using the composition according to the present disclosure, and at least one pixel (pattern) selected from the group consisting of red, green, blue, magenta, yellow, cyan, black, and colorless.

A method for manufacturing the optical filter according to the present disclosure is not particularly limited, and a method including a step of applying the composition according to the present disclosure onto a support to form a composition layer, and a step of curing the composition layer is preferable.

In addition, in a case where the composition according to the present disclosure has pattern forming properties, as a method for manufacturing the optical filter, a method of applying the composition according to the present disclosure onto a support to form a composition layer, curing the composition layer by applying energy in a pattern shape, and removing an uncured portion to which energy is not applied to form a pattern, thereby forming a patterned optical filter can also be used.

In the present disclosure, the infrared cut filter refers to a filter which allows transmission of light (visible light) in the visible range and shields at least a part of light (infrared rays) in the near infrared region. The infrared cut filter may be a filter which allows transmission of light in the entire wavelength range of the visible range, or may be a filter which allows transmission of light in a specific wavelength range of the visible range and shields light in another specific wavelength range of the visible range. In addition, in the present disclosure, a color filter refers to a filter which allows transmission of light in a specific wavelength range of the visible range and shields light in another specific wavelength range of the visible range. In addition, in the present disclosure, an infrared transmitting filter refers to a filter which shields visible light and allows transmission of at least a part of near infrared rays.

### Photothermal Conversion Material

The specific compound (1) according to the present disclosure and the coloring agent composition according to the present disclosure, containing the specific compound (1), have extremely high near infrared absorbing ability, and can be preferably used as a photothermal conversion material.

Examples of the photothermal conversion material include a material for laser welding, and the photothermal conversion material selectively absorbs laser light and locally generates heat, so that the thermoplastic resin as a base material is melted and can be joined.

In addition, the photothermal conversion material can also be used as a material for laser marking, a material for promoting temperature rise, and a drying aid for ink.

### Material for Laser Welding Application

In a case where the specific compound (1) according to the present disclosure and the composition containing the specific compound (1) according to the present disclosure are used for welding a polymer resin, it is possible to join polymer resins with each other by reducing a difference in tone by irradiating with a laser, and to reliably weld contact surfaces with each other to obtain a sufficient bonding strength.

Examples of the polymer resin which can be used for the joining include polystyrene, polymethyl methacrylate, a cycloolefin polymer, polycarbonate, and polyethylene terephthalate.

In recent years, from the viewpoint of weight reduction and cost reduction, polymer resin molded products have been frequently used as components in various fields such as automobile parts. In addition, from the viewpoint of increasing productivity of the polymer resin molded product, a method of dividing the polymer resin molded product in advance into a plurality of parts, molding the divided parts, and joining the molded parts to each other is often adopted.

In the related art, the joining between the polymer resins has been performed by a laser welding method in which a transparent polymer resin having transparency to a laser and an absorbent polymer resin having absorbability to a laser are laminated, and then the transparent polymer resin is irradiated with a laser from the transparent polymer resin side to heat and melt the contact surfaces between the transparent polymer resin and the absorbent polymer resin to integrally bond the transparent polymer resin and the absorbent polymer resin.

Furthermore, in the laser welding method in the related art, in the joining of the same type or different types of polymer resins, the polymer resins to be joined are of two types, one having absorbability to a laser and the other not having absorbability to a laser, so that there is a difference in tone, and there is a limit to the use application of the joined polymer resins.

Specifically, since the polymer resin having low absorption to a laser is a white or transparent laser transmitted color, and the absorbent member is a black laser absorption color such as carbon black, a sense of discomfort in appearance occurs.

That is, in a case where such polymer resins of different colors are joined, there is a problem in that the appearance of the joining force is weak and the joint portion is conspicuous.

In a case of using at least one material selected from the specific compound (1) according to the present disclosure and the composition according to the present disclosure, containing the specific compound (1) according to the present disclosure, since the near infrared absorbing ability of the material is extremely high, these problems can be solved.

In particular, the transparent polymer resins can be joined to each other by using at least one material selected from the specific compound (1) according to the present disclosure and the composition according to the present disclosure, containing the specific compound (1) according to the present disclosure.

For example, in a case where the composition according to the present disclosure, preferably, the composition containing a resin is applied onto a location of a transparent polymer resin to be joined, the applied polymer resin layer is interposed between another transparent polymer resin, and laser irradiation is performed from one side, only the applied location absorbs laser light, locally and instantaneously generates heat, and thus the polymer resins can be melted and joined to each other.

In this case, in a case where the specific compound (1) according to the present disclosure or the composition according to the present disclosure is used, the near infrared absorbing ability is high, and firm joining can be achieved with a small amount of addition, so that the difference in tone of the coated portion is not noticeable.

As another method, a method of kneading the specific compound (1) according to the present disclosure into the transparent polymer resin itself is also considered. The same effect as that of the method of applying the specific compound (1) onto the resin can be expected even in the case of the method of kneading the specific compound (1). That is, in a case where at least one material selected from the specific compound (1) according to the present disclosure or the composition according to the present disclosure, containing the compound, is used for a laser welding application, it is possible to achieve firm joining between desired resins while maintaining high designability.

### Near Infrared Absorbing Compound Represented by General Formula (1)

A first embodiment of the near infrared absorbing compound according to the present disclosure is a near infrared absorbing compound represented by General Formula (1).

The near infrared absorbing compound according to the present disclosure is a novel compound, which has absorption in a long-wavelength infrared region and is thermally stable.

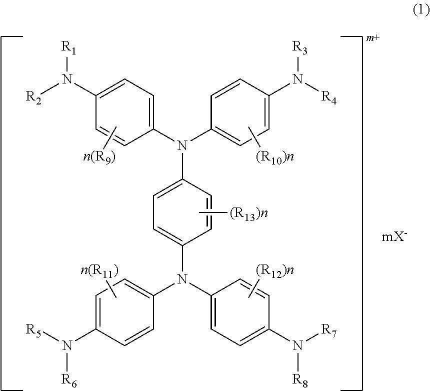

In General Formula (1), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, or a heteroaryl group which may have a substituent, where two or more of R₁, R₂, R₃, R₄, R₅, R₆, R₇, and R₈ may be linked to each other to form a ring,
R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other,
m is 1 or 2, and X⁻ represents a non-nucleophilic anion.

The compound represented by Formula (1) is the same as the specific compound (1) contained in the first embodiment (composition (1)) of the coloring agent composition according to the present disclosure described above; and R₁ to R₈, R₉ to R₁₃, n, m, and X⁻ in General Formula (1) have the same meanings as R₁ to R₈, R₉ to R₁₃, n, and X⁻ in General Formula (1) described above, and preferred examples thereof are also the same.

### Near Infrared Absorbing Compound Represented by General Formula (2)

A second embodiment of the near infrared absorbing compound according to the present disclosure is a near infrared absorbing compound represented by General Formula (2).

The near infrared absorbing compound according to the present disclosure is a novel compound, which has absorption in a long-wavelength infrared region and is thermally stable.

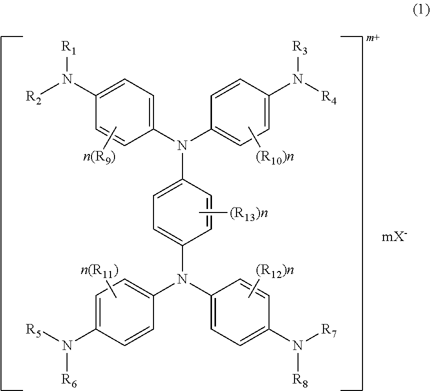

In General Formula (2), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, or a heteroaryl group which may have a substituent, where two or more of R₁, R₂, R₃, R₄, R₅, R₆, R₇, and R₈ may be linked to each other to form a ring,
R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other, and
X⁻ represents a non-nucleophilic anion.

The compound represented by Formula (2) is the same as the specific compound (2) contained in the second embodiment (composition (2)) of the coloring agent composition according to the present disclosure described above; and R₁ to R₈, R₉ to R₁₃, n, and X⁻ in General Formula (2) have the same meanings as R₁ to R₈, R₉ to R₁₃, n, and X⁻ in General Formula (2) described above, and preferred examples thereof are also the same.

The novel near infrared absorbing compound of the present disclosure described above has favorable absorbability in a long-wavelength infrared wavelength region and favorable heat stability, and thus is suitably used for various applications requiring near infrared absorbing property.

Examples of preferred uses are as in the composition according to the present disclosure described above.

### EXAMPLES

Hereinafter, the present disclosure will be described in detail with reference to examples, but the present disclosure is not limited thereto.

In the examples, “%” and “parts” respectively indicate “% by mass” and “parts by mass” unless otherwise specified. In a polymer compound, the molecular weight indicates the weight-average molecular weight (Mw) and the proportion of structural units indicates mole percentage unless otherwise specified.

The weight-average molecular weight (Mw) is a value in terms of polystyrene obtained by performing measurement using a gel permeation chromatography (GPC) method.

### Example 1 and Example 2

According to the following scheme, first, an intermediate X-1 was synthesized, and then Exemplary Compounds (A-1) and (B-1) were synthesized.

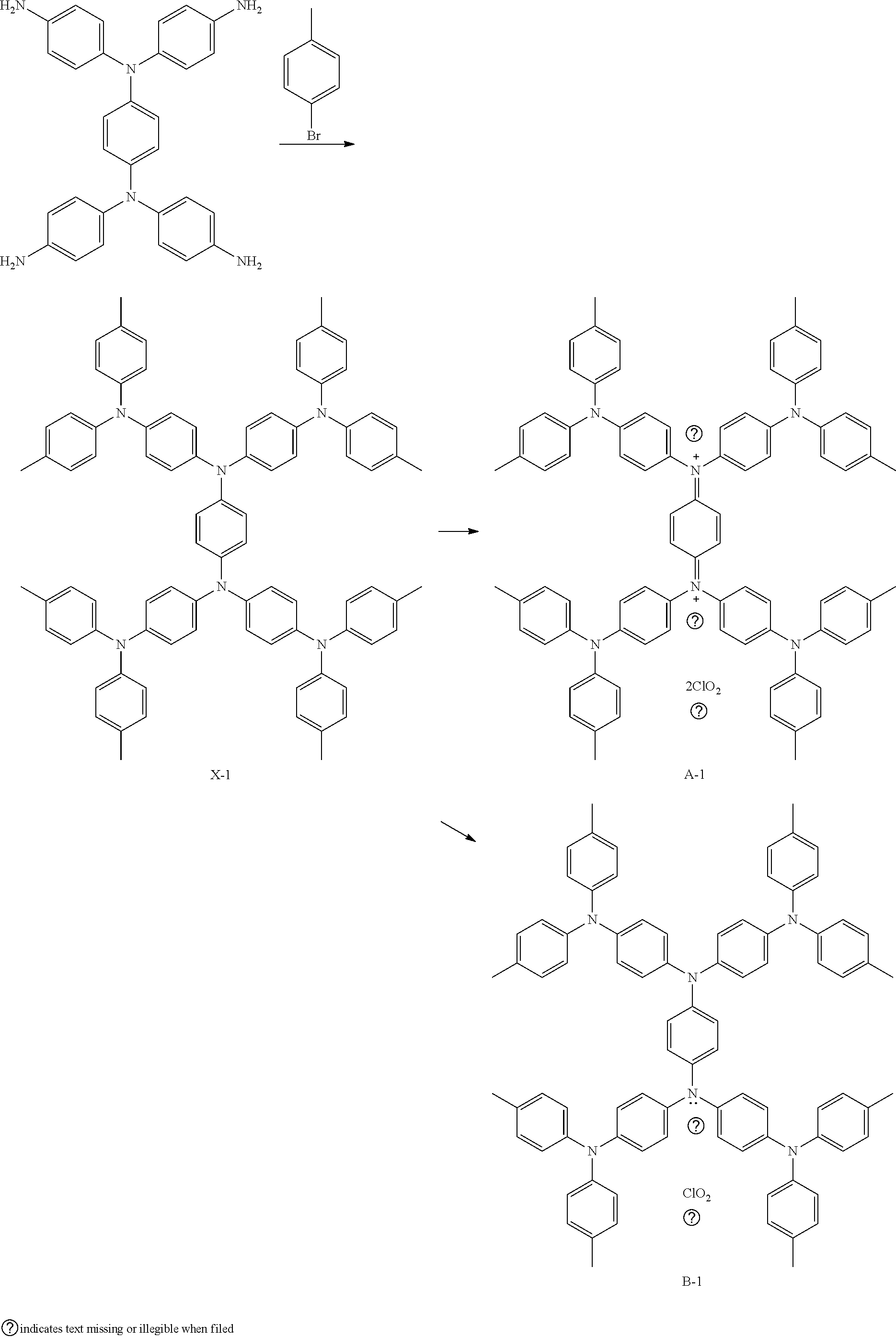

### Synthesis of Intermediate X-1

0.19 g of palladium acetate and 6.5 g (16 equivalents) of sodium tert-butoxide were stirred in 25 ml of toluene at room temperature, 0.34 g (0.4 equivalents) of tri-n-butylphosphine was added thereto, and the reaction solution was heated to 60° C. and stirred for 20 minutes.

2 g of N1,N1′-(1,4-phenylene)bis(N1-(4-aminophenyl)benzene-1,4-diamine) and 8.7 g (12 equivalents) of p-bromotoluene were added to the reaction solution, and the reaction solution was stirred in a reflux state for 3 hours. The reaction solution was cooled to 30° C., 30 ml of a 20% hydrochloric acid aqueous solution and 30 ml of ethyl acetate were added thereto, and the mixture was filtered to obtain a crude product.

The obtained crude product was washed with water and ethyl acetate to obtain 3.4 g of an aromatic amine X-1.

In the proton nuclear magnetic resonance (1H-NMR, solvent: deuterated chloroform (CDC13)), the obtained aromatic amine X-1 had chemical shifts δ of 7.03 (br-s, 22H), 6.96 (br-s, 30H), and 2.30 (s, 24H).

### Example 1: Synthesis of Compound A-1

1 g of the aromatic amine (X-1) obtained above was added to 40 ml of acetonitrile, and 2.6 g (5 equivalents) of Oxone (registered trademark) monopersulfate compound was added thereto as an oxidant, and the reaction solution was stirred at room temperature for 6 hours. 80 ml (5 equivalents) of a 3% sodium chlorate aqueous solution was added to the reaction solution, and the precipitated solid was filtered and washed with water to obtain 1.5 g of a diimmonium compound A-1.

Due to properties of the obtained diimmonium (Exemplary Compound A-1), NMR could not be measured. Mass spectrometry (MS) was performed under the following conditions.

$$MS ( m / z ) = 1291.63 ( {[ {M}^{2 +} , Cl {O}_{4}^{-} ]}^{+} ) , 98.93 ( Cl {O}_{4}^{-} )$$

An absorption maximal wavelength of an absorption spectrum of Exemplary Compound A-1 in a chloroform solution was 1,209 nm.

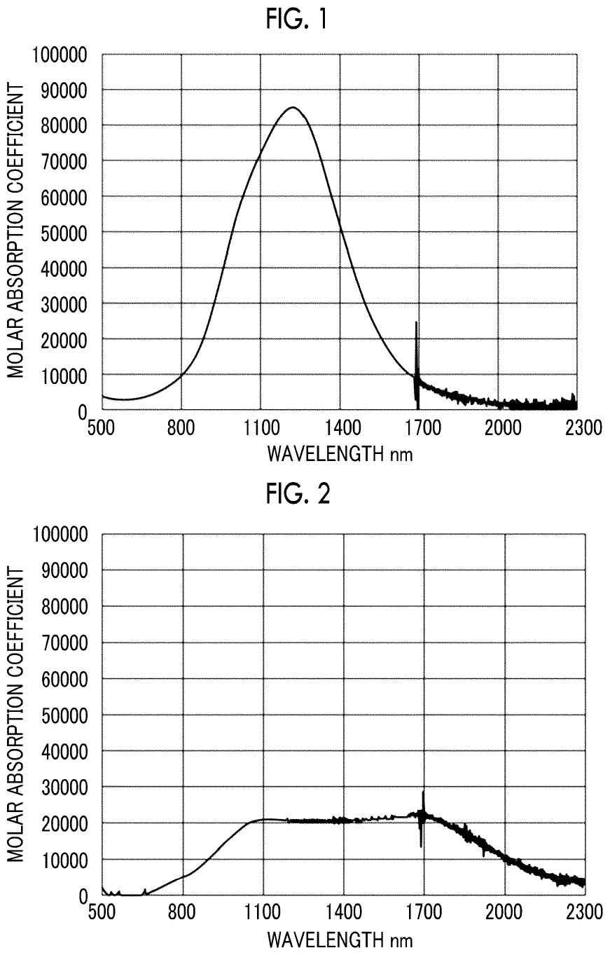

FIG. 1 shows the absorption spectrum of Exemplary Compound A-1 in a chloroform solution.

### Example 2: Synthesis of Compound B-1

1 g of the aromatic amine (X-1) obtained above was added to 40 ml of acetonitrile, and 0.5 g (1 equivalent) of Oxone (registered trademark) monopersulfate compound was added thereto as an oxidant, and the reaction solution was stirred at room temperature for 3 hours. 80 ml (5 equivalents) of a 3% sodium chlorate aqueous solution was added to the reaction solution, and the precipitated solid was filtered and washed with water to obtain 1.6 g of a cationic coloring agent B-1. Due to properties of the obtained cationic coloring agent B-1, NMR could not be measured. Mass spectrometry was performed in the same manner as in Example 1.

$$MS ( m / z ) = 1192.65 ( {M}^{+} ) , 98.93 ( Cl {O}_{4}^{-} )$$

An absorption maximal wavelength of an absorption spectrum of Exemplary Compound (B-1) in a chloroform solution was 1,695 nm.

FIG. 2 shows the absorption spectrum of Exemplary Compound B-1 in a chloroform solution.

### Example 3 and Example 4

First, an intermediate X-3 was synthesized by changing the p-bromotoluene used in the synthesis of the intermediate X-1 to bromomesitylene, and then Exemplary Compounds (A-3) and (B-3) were synthesized.

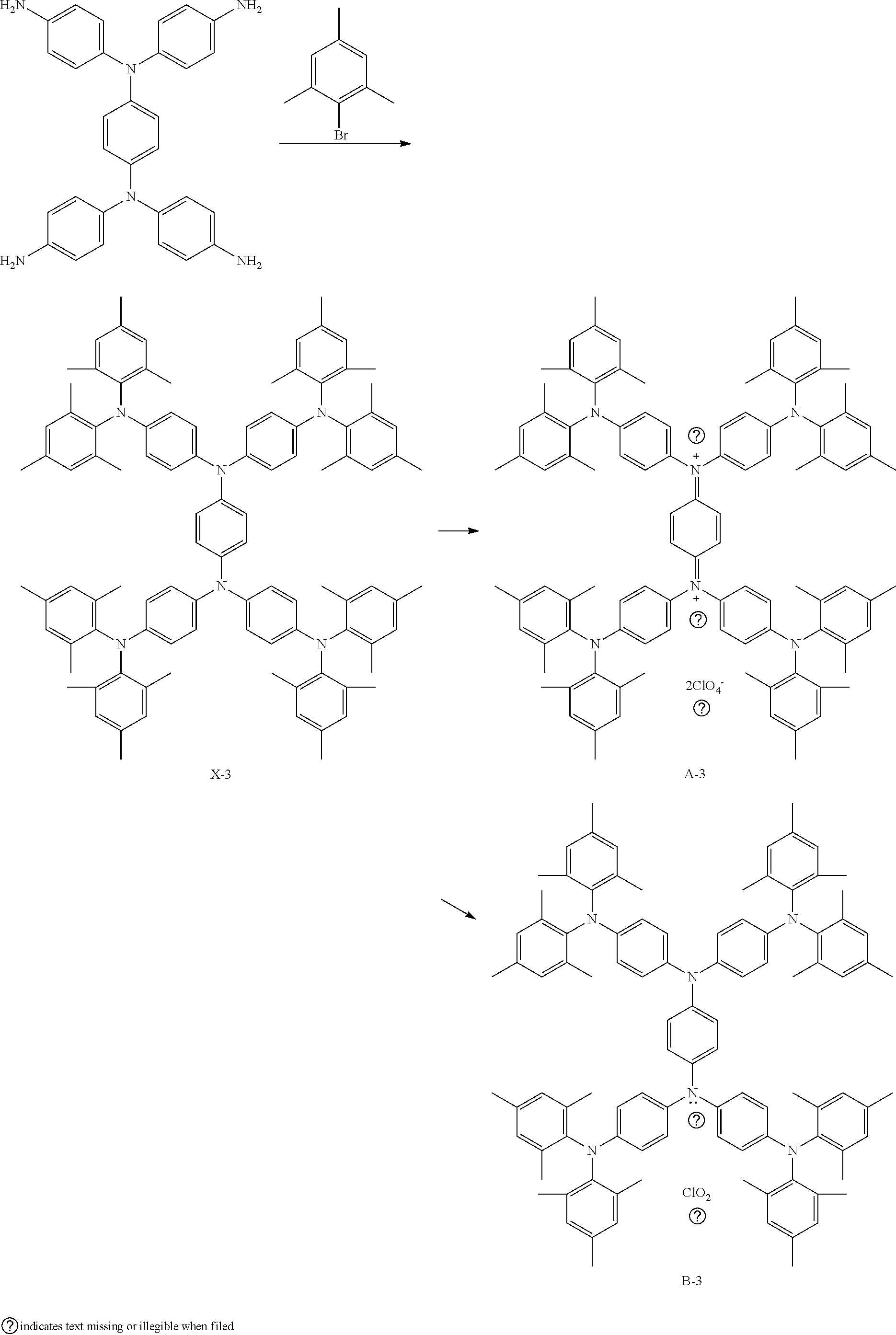

### Synthesis of Intermediate X-3

0.19 g of palladium acetate and 6.5 g (16 equivalents) of sodium tert-butoxide were stirred in 25 ml of toluene at room temperature, 0.34 g (0.4 equivalents) of tri-n-butylphosphine was added thereto, and the reaction solution was heated to 60° C. and stirred for 20 minutes.

2 g of N1,N1′-(1,4-phenylene)bis(N1-(4-aminophenyl)benzene-1,4-diamine) and 10.1 g (12 equivalents) of bromomesitylene were added to the reaction solution, and the reaction solution was stirred in a reflux state for 6 hours. The reaction solution was cooled to 30° C., 30 ml of a 20% hydrochloric acid aqueous solution and 30 ml of ethyl acetate were added thereto, and the mixture was filtered.

The obtained crude product was washed with water and ethyl acetate to obtain 4.0 g of an aromatic amine X-3.

In the proton nuclear magnetic resonance (¹H-NMR, solvent: deuterated chloroform (CDCl₃)), the obtained aromatic amine X-3 had chemical shifts δ of 6.82 (s, 20H), 6.74 (s, 8H), 6.49 (d, J=8.0 Hz, 8H), 2.24 (s, 24H), 1.97 (s, 24H), and 1.73 (s, 24H).

### Example 3: Synthesis of Compound A-3

1 g of the aromatic amine (X-3) obtained above was added to 40 ml of acetonitrile, and 2.2 g (5 equivalents) of Oxone (registered trademark) monopersulfate compound was added thereto as an oxidant, and the reaction solution was stirred at room temperature for 6 hours. 80 ml (5 equivalents) of a 3% sodium chlorate aqueous solution was added to the reaction solution, and the precipitated solid was filtered and washed with water to obtain 1.6 g of a diimmonium compound A-3.

Due to properties of the obtained diimmonium (Exemplary Compound A-3), NMR could not be measured. Mass spectrometry (MS) was performed in the same manner as in Example 1.

$$MS ( m / z ) = 1516.81 ( {[ {M}^{2 +} , Cl {O}_{4}^{-} ]}^{+} ) , 98.94 ( Cl {O}_{4}^{-} )$$

An absorption maximal wavelength of an absorption spectrum of Exemplary Compound A-3 in a chloroform solution was 1,231 nm.

### Example 4: Synthesis of Compound B-3

1 g of the aromatic amine (X-1) obtained above was added to 40 ml of acetonitrile, and 0.4 g (1 equivalent) of Oxone (registered trademark) monopersulfate compound was added thereto as an oxidant, and the reaction solution was stirred at room temperature for 3 hours. 80 ml (5 equivalents) of a 3% sodium chlorate aqueous solution was added to the reaction solution, and the precipitated solid was filtered and washed with water to obtain 1.6 g of a cationic coloring agent B-1. Due to properties of the obtained cationic coloring agent B-1, NMR could not be measured. Mass spectrometry was performed in the same manner as in Example 1.

$$MS ( m / z ) = 1417.85 ( {M}^{+} ) , 98.93 ( Cl {O}_{4}^{-} )$$

An absorption maximal wavelength of an absorption spectrum of Exemplary Compound (B-3) in a chloroform solution was 1,695 nm.

### Example 5 and Example 6

Exemplary Compounds (A-35) and (B-35) were synthesized by changing the 3% sodium chlorate aqueous solution used in the synthesis of Exemplary Compounds (A-1) and (B-1) to a 3% lithium tetrakis(pentafluorophenyl)borate aqueous solution.

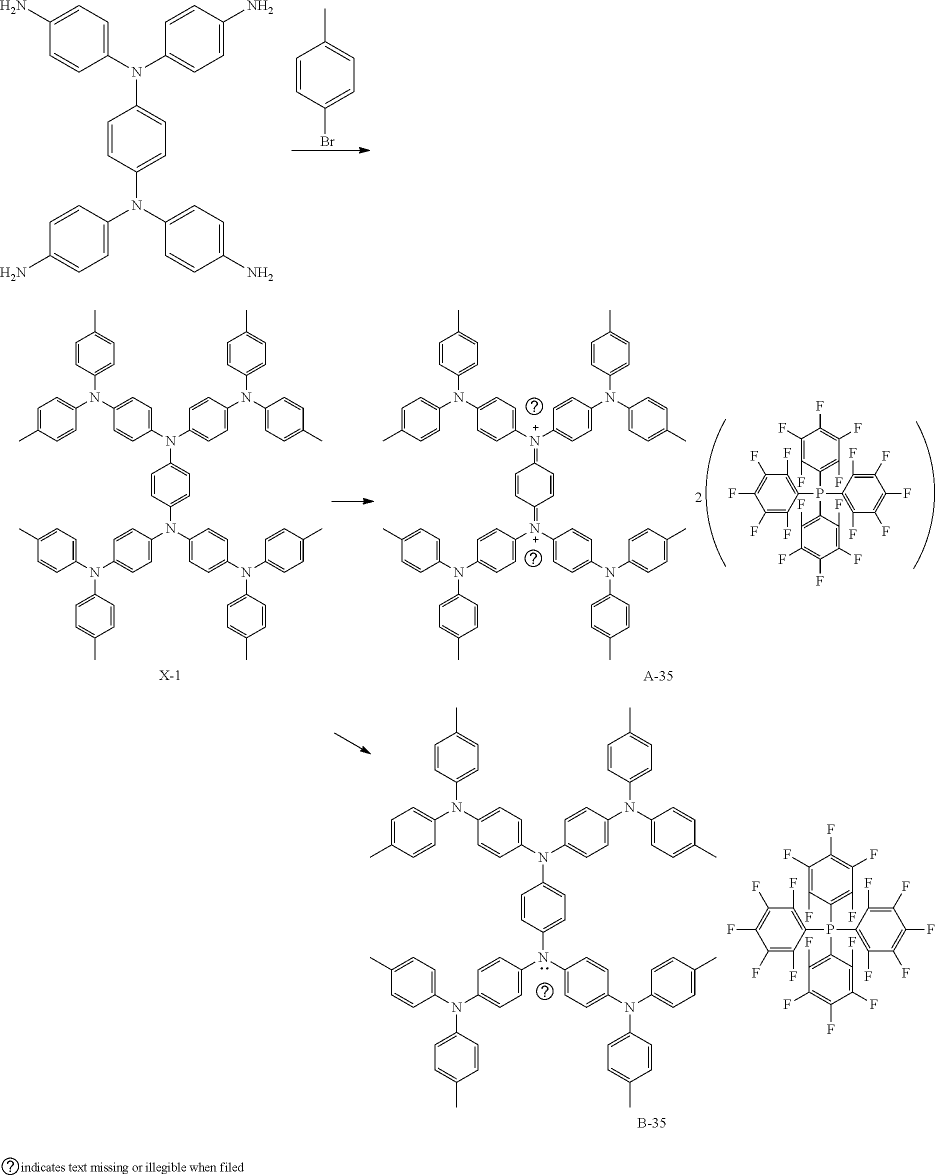

### Example 5: Synthesis of Compound A-35

1 g of the aromatic amine (X-1) was added to 40 ml of acetonitrile, and 2.2 g (5 equivalents) of Oxone (registered trademark) monopersulfate compound was added thereto as an oxidant, and the reaction solution was stirred at room temperature for 6 hours. 80 ml (5 equivalents) of a 3% lithium tetrakis (pentafluorophenyl) borate aqueous solution was added to the reaction solution, and the precipitated solid was filtered and washed with water to obtain 2.0 g of a diimmonium compound A-3.

Due to properties of the obtained diimmonium (Exemplary Compound A-35), NMR could not be measured. Mass spectrometry (MS) was performed in the same manner as in Example 1.

$$MS ( m / z ) = 1872.59 ( {[ {M}^{2 +} , B {( {C}_{6} {F}_{5} )}_{4}^{-} ]}^{+} ) , {678.99 [ {B ( {C}_{6} {F}_{5} )}_{4} ]}^{-}$$

An absorption maximal wavelength of an absorption spectrum of Exemplary Compound A-35 in a chloroform solution was 1,338 nm.

### Example 6: Synthesis of Compound B-35

1 g of the aromatic amine (X-1) obtained above was added to 40 ml of acetonitrile, and 0.4 g (1 equivalent) of Oxone (registered trademark) monopersulfate compound was added thereto as an oxidant, and the reaction solution was stirred at room temperature for 3 hours. 80 ml (5 equivalents) of a 3% sodium chlorate aqueous solution was added to the reaction solution, and the precipitated solid was filtered and washed with water to obtain 1.6 g of a cationic coloring agent B-1. Due to properties of the obtained cationic coloring agent B-1, NMR could not be measured. Mass spectrometry was performed in the same manner as in Example 1.

$$MS ( m / z ) = 1192.63 ( {M}^{+} ) , {678.98 [ B {( {C}_{6} {F}_{5} )}_{4} ]}^{-}$$

An absorption maximal wavelength of an absorption spectrum of Exemplary Compound (B-35) in a chloroform solution was 1,821 nm.

### Example 7 to Example 42

In the same manner, the above-described compounds represented by General Formulae (1) and (2) could be synthesized by appropriately changing the p-bromotoluene used in the synthesis of the aromatic amine (X-1) as the intermediate to a corresponding brominated aryl compound in a case of changing R₁ to R₈, and could be synthesized by appropriately changing the 3% sodium chlorate aqueous solution used in the synthesis of A-1 and B-1 in a case of changing X.

Specific compounds shown in Tables 1 and 2 were obtained according to the above scheme.

### Comparative Example 1 to Comparative Example 6

The following known diimmonium compounds as near infrared absorbing compounds were used as comparative compounds 1 to 6.

Comparative compound 1: a comparative compound 1 was obtained according to the description in JP2009-180875A.

Comparative compound 2: a comparative compound 2 was obtained according to the description in JP2017-116775A.

Comparative compound 3: a comparative compound 3 was obtained according to the description in JP2002-275134A

Comparative compound 4: a diimmonium compound (product code T3072; the following structure) manufactured by Tokyo Chemical Industry Co., Ltd. was used as a comparative compound 4.

Comparative compound 5: a comparative compound 5 was obtained according to the description in JP2006-188653A

Comparative compound 6: a comparative compound 6 was obtained according to the description in JP2016-45391A.

Structures of the respective comparative compounds are shown below.

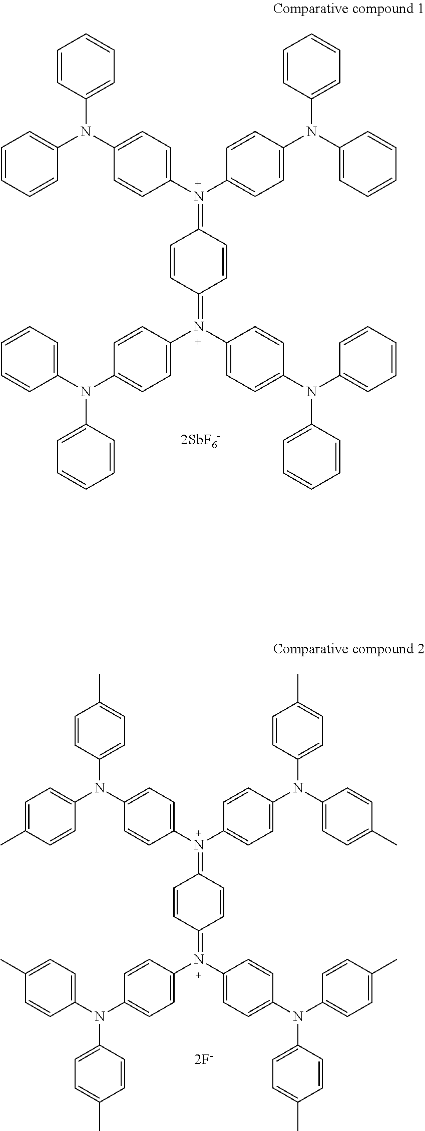

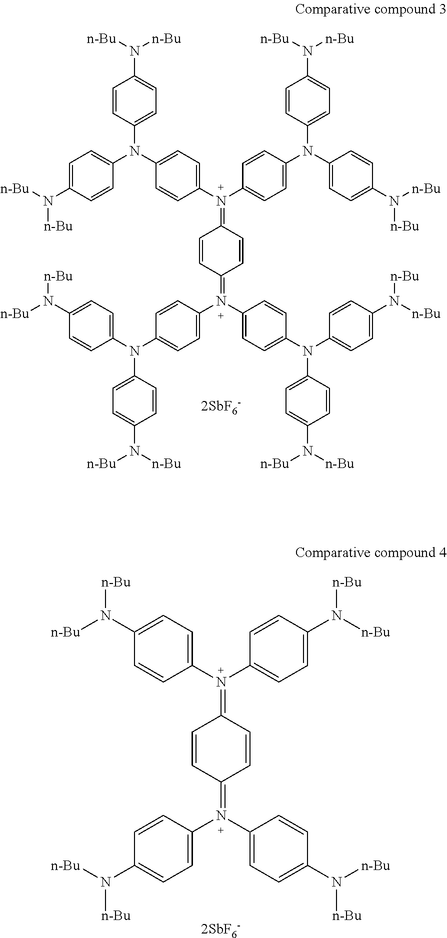

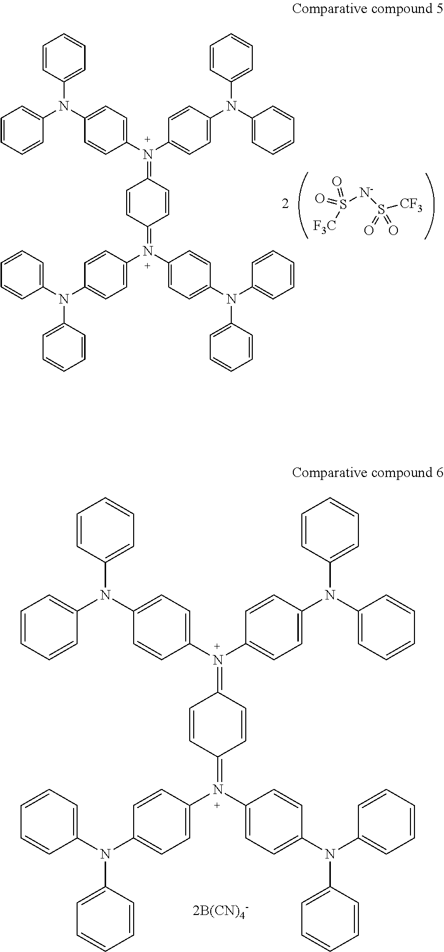

### Evaluation of Specific Compound

The obtained specific compounds and comparative compounds 1 to 6 were evaluated by the following methods.

The results are shown in Tables 1 and 2 below.

1. Mass Spectrometry of Compound by Matrix-Assisted Laser Desorption/Ionization-Mass Spectrometry (hereinafter, referred to as “MALDI-MS”)

Each of the compounds shown in Tables 1 and 2 below was dissolved in chloroform to obtain a mixture of a matrix chloroform solution, 1 mL (liter) of the mixture was applied onto a plate in a spot manner, the liquid droplets were air-dried, and MALDI-MS was measured with the following device.
Device: UltrafleXtreme manufactured by Bruker Corporation; ionization: posi., nega.; measurement mode: reflectron; matrix: DCTB

It was confirmed that the compounds shown in Tables 1 and 2 were each of Exemplary Compounds described above or the comparative compounds described above.

### 2. Evaluation of Absorption Spectrum

For each of Exemplary Compounds shown in Tables 1 and 2, a sample solution was prepared by diluting the compound with chloroform to a concentration of 3.0×10⁻⁵ mol/L, and near infrared absorbability was evaluated.

For each sample solution, an absorbance was measured in a 1 mm quartz cell using a spectrophotometer (UV-3600 Plus, manufactured by Shimadzu Corporation).

A maximal absorption wavelength (Amax) was measured from the absorption spectrum of each sample solution. The results are shown in Tables 1 and 2, and the wavelength of Amax was evaluated according to the following standard.

### (Evaluation Standard)

A: Amax was 1,150 nm or more.
B: Amax was less than 1,150 nm.

### 3. Evaluation of Solubility

For each of Exemplary Compounds shown in Tables 1 and 2, a predetermined amount of the compound was added to a solvent of methyl ethyl ketone (MEK), and solubility was evaluated according to the following standard by stirring the mixture at room temperature for 30 minutes.

### (Evaluation Standard)

A: 1 g or more of the compound was stably dissolved in 300 g of MEK.
B: 1 g was not dissolved in 300 g of MEK.
X: decomposition was observed by measuring the absorption spectrum after the dissolution.

### 4. Evaluation of Heat Stability

30 mg of each of Exemplary Compounds and the comparative compounds described in the tables was dissolved in 8.9 g of methyl ethyl ketone and 1.1 g of a polystyrene resin (manufactured by Sigma-Aldrich Co. LLC) to prepare a resin composition.

The obtained resin composition was spin-coated on a glass substrate to form a coating film, and the obtained coating film was dried at 60° C. for 2 minutes to produce a resin film.

The resin film produced on the glass substrate was placed on a hot plate at 180° C. for 5 minutes, and an absorbance reduction rate of Amax in the heat resistance test of the film was measured and evaluated according to the following standard.

### (Evaluation Standard)

A: absorbance reduction rate of Amax was less than 5%.
B: absorbance reduction rate of Amax was 5% or more and less than 10%.
C: absorbance reduction rate of Amax was 10% or more and less than 50%.
D: absorbance reduction rate of Amax was 50% or more.
X: solubility was insufficient, so that the film cannot be produced, which was not evaluable.

TABLE 1

| | | | Near | | Heat |
|---|---|---|---|---|---|
| | | | infrared | | stability |
| | Exemplary | λmax | absorbing | Solubility | of resin |
| | compound | (nm) | property | in solvent | composition |
| Example 1 | A-1 | 1209 | A | A | A |
| Example 2 | B-1 | 1695 | A | A | A |
| Example 3 | A-3 | 1231 | A | A | A |
| Example 4 | B-3 | 1417 | A | A | A |
| Example 5 | A-35 | 1338 | A | A | A |
| Example 6 | A-35 | 1821 | A | A | A |
| Example 7 | A-6 | 1248 | A | A | A |
| Example 8 | B-6 | 1722 | A | A | A |
| Example 9 | A-7 | 1162 | A | A | A |
| Example 10 | B-7 | 1630 | A | A | A |
| Example 11 | A-8 | 1210 | A | A | A |
| Example 12 | B-8 | 1700 | A | A | A |
| Example 13 | A-9 | 1170 | A | A | A |
| Example 14 | B-9 | 1633 | A | A | A |
| Example 15 | A-11 | 1282 | A | A | A |
| Example 16 | B-11 | 1743 | A | A | A |
| Example 17 | A-13 | 1211 | A | A | A |
| Example 18 | B-13 | 1731 | A | A | A |
| Example 19 | A-19 | 1165 | A | A | A |
| Example 20 | B-19 | 1629 | A | A | A |
| Example 21 | A-22 | 1308 | A | A | A |
| Example 22 | B-22 | 1787 | A | A | A |
| Example 23 | A-25 | 1299 | A | A | A |
| Example 24 | B-25 | 1754 | A | A | A |
| Example 25 | A-26 | 1232 | A | A | A |
| Example 26 | B-26 | 1727 | A | A | A |

TABLE 2

| | Exemplary | | Near | | Heat |
|---|---|---|---|---|---|
| | compound | | infrared | Solubility | stability |
| | or comparative | λmax | absorbing | in | of resin |
| | compound | (nm) | property | solvent | composition |
| Example 27 | A-28 | 1209 | A | A | A |
| Example 28 | B-28 | 1695 | A | A | A |
| Example 29 | A-29 | 1209 | A | A | A |
| Example 30 | B-29 | 1695 | A | A | A |
| Example 31 | A-30 | 1209 | A | A | A |
| Example 32 | B-30 | 1695 | A | A | A |
| Example 33 | A-34 | 1211 | A | A | A |
| Example 34 | B-34 | 1700 | A | A | A |
| Example 35 | A-36 | 1215 | A | A | A |
| Example 36 | B-36 | 1711 | A | A | A |
| Example 37 | A-37 | 1209 | A | A | A |
| Example 38 | B-37 | 1695 | A | A | A |
| Example 39 | A-38 | 1209 | A | A | A |
| Example 40 | B-38 | 1695 | A | A | A |
| Example 41 | A-39 | 1209 | A | A | A |
| Example 42 | B-39 | 1695 | A | A | A |
| Comparative | Comparative | 1230 | A | B | X |
| Example 1 | compound 1 | | | | |
| Comparative | Comparative | 1250 | A | X | D |
| Example 2 | compound 2 | | | | |
| Comparative | Comparative | 1051 | B | X | D |
| Example 3 | compound 3 | | | | |
| Comparative | Comparative | 1100 | B | A | D |
| Example 4 | compound 4 | | | | |
| Comparative | Comparative | 1230 | A | B | X |
| Example 5 | compound 5 | | | | |
| Comparative | Comparative | 1230 | A | B | X |
| Example 6 | compound 6 | | | | |

All of the specific compounds of Examples had absorption in the near infrared wavelength region, and had favorable near infrared absorbing property and solubility in a solvent.

In addition, it was found that all of the resin compositions containing the specific compound had favorable heat stability.

On the other hand, even in the comparative compound 1, the comparative compound 5, and the comparative compound 6, in which the diimmonium skeleton had an aryl group but did not have a substituent, the solubility in a solvent was inferior to the specific compounds of Examples, so that it was not possible to prepare a uniform resin composition and form a cured film, and thus the heat stability could not be evaluated.

In addition, the comparative compound 3 having a substituent other than the substituent in which a σp value on Hammett's rule was −0.5 or more in the aryl group in R₁ to R₈ was decomposed by the measurement of the absorption spectrum after dissolving the comparative compound 3 in a solvent, and thus the stability was low.

In the comparative compound 4 in which R₁ to R₈ were substituents other than an aryl group and a heteroaryl group, the solubility in a solvent was favorable, but the absorbance at the maximal absorption wavelength was significantly reduced by heating at 130° C., and thus the heat stability of the resin composition was low.

The comparative compound 2 in which the counter anion X⁻ was a nucleophilic fluoride ion was decomposed by the measurement of the absorption spectrum after dissolving the comparative compound 2 in a solvent, and thus the stability was low.

### Example 43 to Example 49 and Comparative Example 7 to Comparative Example 9

A resin composition, which was a kneaded material of the specific compound or the comparative compound and a resin, was prepared, and the following kneading evaluation was performed.

### 5. Evaluation of Kneading

The specific compounds and comparative compounds shown in Table 3 below were weighed in the contents shown in Table 3, and a sample obtained by shaking and mixing 12.0 g of the resin shown in Table 3 was prepared. A polycarbonate resin (indicated as Polycarbonate in Table 3), a polystyrene resin, and an acrylic resin were used as the resin. In Table 3, the polycarbonate resin is indicated as Polycarbonate, the polystyrene resin is indicated as Polystyrene, and the acrylic resin is indicated as Acrylic.

The obtained sample was put into a supply port of a biaxial kneader (Labo Plastomill, manufactured by Toyo Seiki Seisaku-sho, Ltd.) and subjected to melt-kneading to obtain a resin composition as a kneaded material. The obtained resin composition was pressed using a heat press machine to obtain a resin film having a thickness of 0.15 mm.

In a case where a polycarbonate resin (SD PORYCA (registered trademark) 301-10, manufactured by Sumika Polycarbonate Co., Ltd.) was used as the resin, the melt-kneading temperature was set to 260° C. and the hot press temperature was set to 230° C.

In a case where a polystyrene resin (manufactured by Sigma-Aldrich Co. LLC) was used as the resin, the melt-kneading temperature was set to 200° C. and the hot press temperature was set to 180° C.

In a case where an acrylic resin (G1000, manufactured by Kuraray Co., Ltd.) was used as the resin, the melt-kneading temperature was set to 180° C. and the hot press temperature was set to 160° C.

A transmittance of the obtained resin film at a wavelength of 1,150 nm was measured with a spectrophotometer, and the measured value was evaluated according to the following standard. In Table 3, it is described in “Evaluation of transmittance at wavelength of 1150 nm”. As the transmittance is lower, the near infrared blocking property is better. The results are shown in Table 3.

### (Evaluation Standard)

A: transmittance at a wavelength of 1,150 nm was less than 10%.
B: transmittance at a wavelength of 1,150 nm was 10% or more and less than 50%.
C: transmittance at a wavelength of 1,150 nm was 50% or more.

TABLE 3

| | Exemplary | | | | |
|---|---|---|---|---|---|
| | compound or | | Evaluation of transmittance | | |
| | comparative | Content | at wavelength of 1150 nm | | |
| | compound | (mg) | Polycarbonate | Polystyrene | Acrylic |
| Example 43 | A-28 | 18 | B | A | A |
| Example 44 | A-31 | 19 | A | A | A |
| Example 45 | A-34 | 22 | A | A | A |
| Example 46 | A-35 | 28 | A | A | A |
| Example 47 | A-36 | 20 | A | A | A |
| Example 48 | A-40 | 18 | B | A | A |
| Example 49 | A-44 | 26 | A | A | A |
| Comparative | Comparative | 14 | C | C | C |
| Example 7 | compound 2 | | | | |
| Comparative | Comparative | 30 | C | C | C |
| Example 8 | compound 3 | | | | |
| Comparative | Comparative | 16 | C | C | C |
| Example 9 | compound 4 | | | | |

Since all of the specific compounds of Examples had high compatibility with the resin and were thermally stable, the resin film formed of the resin composition, which was a kneaded material obtained by kneading the resin at a temperature of 180° C. to 240° C., had favorable near infrared shielding property.

On the other hand, since all of the comparative compound 2 in which the counter anion X⁻ was a nucleophilic fluoride ion even in a case where the diimmonium skeleton had an aryl group, the comparative compound 3 having a substituent other than the substituent in which a σp value on Hammett's rule was −0.5 or more in the aryl group in R₁ to R₈, and the comparative compound 4 in which R₁ to R₈ were substituents other than an aryl group and a heteroaryl group had low heat stability, the films of the resin compositions, which were a kneaded material with the resin obtained under the above-described thermal conditions, are all inferior in near infrared shielding property.

The disclosure of JP2023-056456 filed on Mar. 30, 2023 and the disclosure of JP2023-166087 filed on Sep. 27, 2023 are incorporated in the present disclosure by reference.

All documents, patent applications, and technical standards described in the present disclosure are incorporated herein by reference to the same extent as in a case of being specifically and individually noted that individual documents, patent applications, and technical standards are incorporated by reference.

What is claimed is:

1. A coloring agent composition comprising:
a compound represented by General Formula (1), 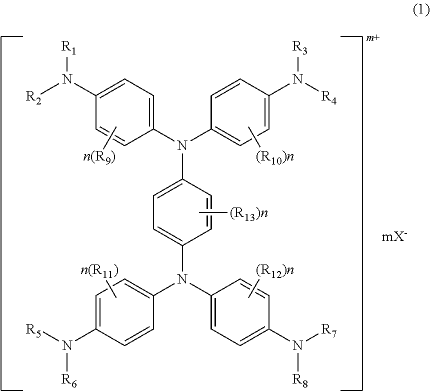
in General Formula (1), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring,
R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other,
m is 1 or 2, and X⁻ represents a non-nucleophilic anion.

2. A coloring agent composition comprising:
a compound represented by General Formula (2), 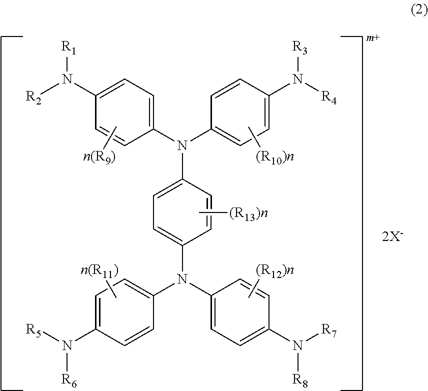
in General Formula (2), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring,
R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other, and
X⁻ represents a non-nucleophilic anion.

3. The coloring agent composition according to claim 1,
wherein the coloring agent composition is an image forming material.

4. The coloring agent composition according to claim 1, further comprising:
a resin.

5. A film comprising:
the coloring agent composition according to claim 4.

6. An optical filter comprising:
the film according to claim 5.

7. A near infrared absorbing compound represented by General Formula (1), 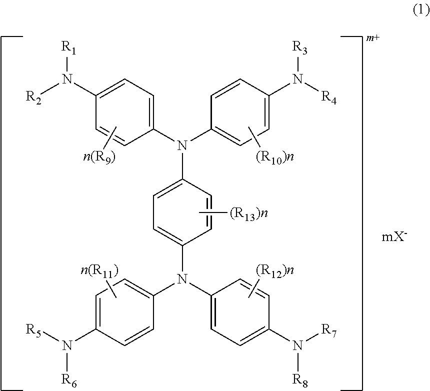
in General Formula (1), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring,
R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other,
m is 1 or 2, and X represents a non-nucleophilic anion.

8. A near infrared absorbing compound represented by General Formula (2), 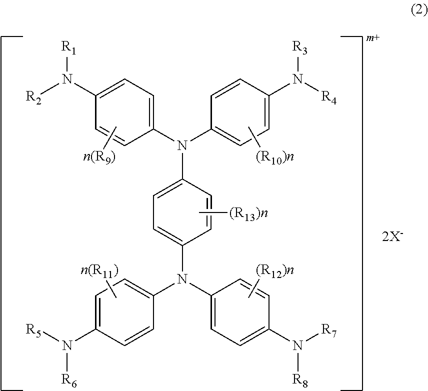
in General Formula (2), R₁, R₂, R₃, R₄, R₅, R₆, R₇, and R₈ each independently represent a group selected from a phenyl group having a substituent in which a σp value on Hammett's rule is −0.5 or more, an aryl group having 7 to 20 carbon atoms, which may have a substituent, or a heteroaryl group which may have a substituent, where R₁, R₂, R₃, R₄, R₅, R₆, R₇, and R₈ may be the same or different from each other, and two or more thereof may be linked to each other to form a ring,
R₉, R₁₀, R₁₁, R₁₂, and R₁₃ each independently represent a monovalent substituent, five n's each independently represent an integer of 0 to 4, in a case where n is an integer of 2 to 4, a plurality of R₉'s, R₁₀'s, R₁₁'s, R₁₂'s, and R₁₃'s may be the same or different from each other, and
X⁻ represents a non-nucleophilic anion.
